(19) United States
(12) Patent Application Publication
(10) Pub. No.: US 2016/0068630 A1
(43) Pub. Date: Mar. 10, 2016

(54) Process for Preparing Polycarbonates by Polymerization of Five-Membered-Ring Cyclic Carbonates

(71) Applicants: TOTAL RESEARCH & TECHNOLOGY FELUY, Seneffe (BE); CENTRE NATIONAL DE LA RECHERCHE SCIENTIFIQUE (CNRS), Paris (FR)

(72) Inventors: Jean-Francois CARPENTIER, Acigne (FR); Sophie GUILLAUME, Vitre (FR); William GUERIN, Rennes (FR)

(21) Appl. No.: 14/786,227

(22) PCT Filed: Apr. 29, 2014
(86) PCT No.: PCT/EP2014/058716

§ 371 Date: Oct. 22, 2015

(30) Foreign Application Priority Data
Apr. 29, 2013 (EP) 13290095.2

(51) Int. Cl. C08G 64/30; C08G 63/64; C07D 317/36; C07D 317/46

(52) U.S. Cl. CPC C08G 64/305; C07D 317/46; C08G 63/64; C07D 317/36

(57) ABSTRACT

A process for the preparation of polycarbonates may include ring-opening polymerization of five-membered cyclic carbonates in the presence of a catalyst; a compound of formula (I), stereoisomers, racemics, or mixtures thereof; and optionally cyclic esters, stereoisomers, racemics, or mixtures thereof. Formula (I) is:

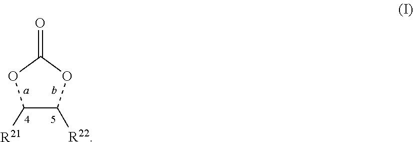

Each dotted bond “a” and “b” is a solid bond, a wedged bond, or a hashed wedged bond. R²¹ and R²², taken together with the carbon atoms 4 and 5 of the cyclic carbonate, may form a ring. The ring may be a C₅₋₉cycloalkylene, C₅₋₉cycloalkenylene, or C₆₋₁₂arylene. The ring may optionally be substituted with substituents including halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

### FIELD OF THE INVENTION

The invention relates to a process for the preparation of polycarbonates or copolymers thereof. In particular the invention relates to a process for the preparation of polycarbonates, or copolymers thereof, by ring opening polymerization of five-membered ring cyclic carbonates.

### BACKGROUND OF THE INVENTION

Polycarbonates are becoming more important as alternatives to synthetic polymers. There already exist commercially available polycarbonates with excellent electrical insulating properties, transparency, and heat resistance and polycarbonates are present in many products, like adhesives for compact discs, in packaging, and optical lenses, to name a few.

One of the most widely used polycarbonates is bisphenol A polycarbonate (BPA-PC), which has interesting thermal and mechanical properties but has recently been reported as being a potential hazard in food/human contact applications. Other commercially available polycarbonates such as propylene polycarbonate (PPC), ethylene polycarbonate (PEC), poly(butylene carbonate) (PBC), or poly(trimethylene carbonate) (PTMC), while presenting particular thermo-mechanical properties, still do not provide a durable and attractive alternative, especially as commodity polymers for packaging or as elastomers.

One alternative to bisphenol A polycarbonate is poly(cyclohexene carbonate) (PCHC). PCHC has a melting temperature of 115° C., lower than that of BPA-PC (150° C.) and a degradation temperature of ca. 310° C., similar to that of BPA-PC (300° C.). It has an elongation at break (εᵣ) of 2% and a Young's modulus (E) of 3500 MPa, significantly higher than that of BPA-PC (2400 MPa). Due to these properties, PCHC is a promising replacer for BPA-PC.

Since the late 90s different groups have synthesized PCHC by copolymerizing CO₂ with cyclohexene oxide. This synthesis presents problems in controlling the stereochemistry and the chemoselectivity of the PCHC, such that an ether-free polycarbonate sequence is difficult to obtain.

Consequently, it is an object of the present invention to provide an improved process for the polymerization of five-membered cyclic carbonates. It is also an object of the present invention to provide an improved process for the copolymerization of five-membered cyclic carbonates with cyclic esters. It is an object of the present invention to provide novel polycarbonates, particularly poly(cyclic carbonates), more particularly PCHC, with improved mechanical properties.

### SUMMARY OF THE INVENTION

The present inventors have now found that any one of the objects above can be attained by using the processes as presently claimed.

According to a first aspect of the present invention, a process for preparing a polycarbonate or a copolymer thereof is provided. The process for preparing a polycarbonate or a copolymer thereof comprises the step of homo- or copolymerizing in the presence of at least one catalyst, one or more compounds of formula (I), stereoisomers, racemics, or mixtures thereof; optionally with one or more cyclic esters, stereoisomers, racemics, or mixtures thereof;

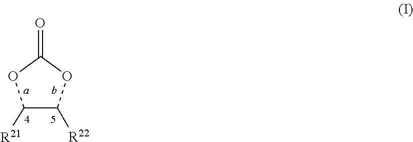

wherein
each dotted bond “a” and “b” independently represents a solid bond, a wedged bond, or a hashed wedged bond;
each R²¹ and R²² are, independently, selected from C₁₋₆alkyl, C₃₋₆cycloalkyl, and C₆₋₁₀aryl; or R²¹ and R²² taken together with the carbon atoms 4 and 5 of the cyclic carbonate form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

Preferably, the present invention provides a process for preparing a polycarbonate or copolymer thereof, said process comprising copolymerizing in the presence of at least one catalyst, one or more compounds of formula (I), stereoisomers, racemics, or mixtures thereof; with one or more cyclic esters, stereoisomers, racemics, or mixtures thereof;

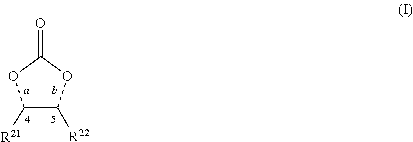

wherein
each dotted bond “a” and “b” independently represents a solid bond, a wedged bond, or a hashed wedged bond; and
R²¹ and R²² taken together with the carbon atoms 4 and 5 of the cyclic carbonate form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; wherein two of said one or more substituents can join to form a ring fused to said C₃₋₉cycloalkylene, C₅₋₉cycloalkenylene, or C₆₋₁₂arylene; wherein said ring formed by said two substituents is selected from the group comprising C₃₋₉cycloalkyl, C₅₋₉cycloalkenyl, C₆₋₁₂aryl, and heterocyclyl, wherein each group can be unsubstituted or substituted with one or more R⁴⁰ wherein each R⁴⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

More preferably, the present invention provides a process for preparing a polycarbonate or copolymer thereof, said process comprising homo- or copolymerizing in the presence of at least one catalyst, one or more compounds of formula (I), stereoisomers, racemics, or mixtures thereof; optionally with one or more cyclic esters, stereoisomers, racemics, or mixtures thereof;

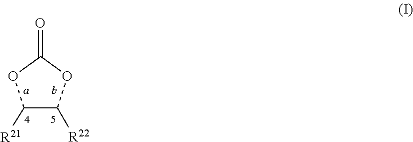

wherein
each dotted bond “a” and “b” independently represents a solid bond, a wedged bond, or a hashed wedged bond; and
R²¹ and R²² taken together with the carbon atoms 4 and 5 of the cyclic carbonate form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

According to a second aspect, the present invention further encompasses a process for the synthesis of a compound of formula (I) as defined above, a stereoisomer or racemate thereof, comprising the step of contacting a compound of formula (V), a stereoisomer, or racemate thereof; with at least one reagent selected from the group consisting of an alkyl- or cycloalkyl-chloroformate derivative of formula (VI); an urea; phosgene or a derivative/substitute of phosgene, e.g. triphosgene; a cyclic carbonate; and carbon dioxide;

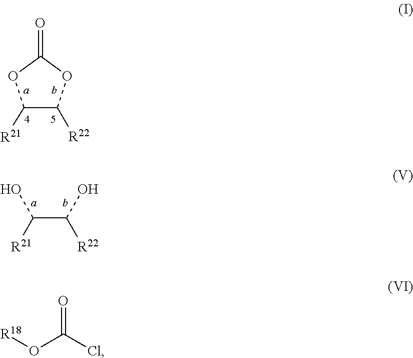

wherein
each dotted bond “a” and “b” independently represents a solid bond, a wedged bond, or a hashed wedged bond;
R²¹ and R²² are each independently selected from C₁₋₆alkyl, C₃₋₆cycloalkyl, and C₆₋₁₀aryl; or R²¹ and R²² taken together with the carbon atoms to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; and
R¹⁸ is C₁₋₆alkyl or C₃₋₆cycloalkyl.

Preferably, the present invention further provides a process for the synthesis of a compound of formula (I) a stereoisomer or racemate thereof, comprising the step of contacting a compound of formula (V), a stereoisomer, or racemate thereof; with at least one reagent selected from the group consisting of an alkyl- or cycloalkyl-chloroformate of formula (VI); an urea; a phosgene or derivative thereof; a cyclic carbonate; and carbon dioxide;

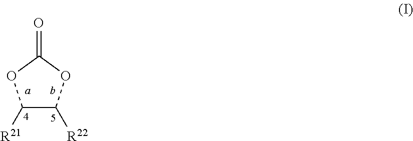

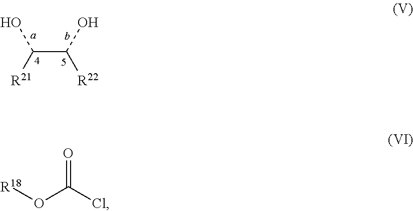

wherein
each dotted bond “a” and “b” independently represents a solid bond, a wedged bond, or a hashed wedged bond;
R²¹ and R²² taken together with the carbon atoms to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; and
R¹⁸ is C₁₋₆alkyl, or C₃₋₆cycloalkyl.

According to a third aspect, the present invention also encompasses a polycarbonate or copolymer thereof comprising a recurring unit of formula (VII1) and/or (VII2),

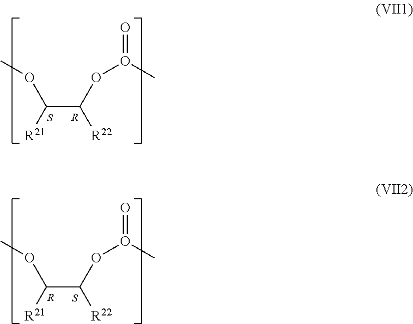

wherein
R²¹ and R²² are each independently selected from C₁₋₆alkyl, C₃₋₆cycloalkyl, and C₆₋₁₀aryl; or R²¹ and R²² taken together with the carbon atoms 4 and 5 to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

Preferably, the present invention also encompasses a polycarbonate or copolymer thereof comprising a recurring unit of formula (VII1) and/or (VII2),

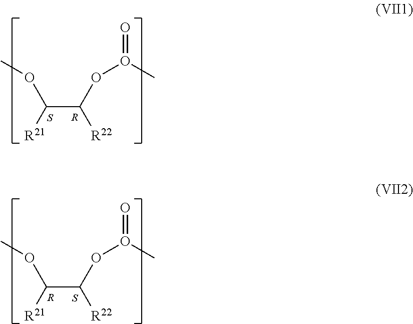

wherein
R²¹ and R²² taken together with the carbon atoms to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

According to a fourth aspect, the present invention also encompasses a polycarbonate or copolymer thereof comprising recurring unit of formula (VII3) or (VII4),

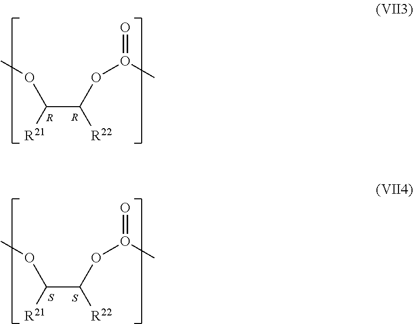

wherein
R²¹ and R²² are each independently selected from C₁₋₆alkyl, C₃₋₆cycloalkyl, and C₆₋₁₀aryl; or R²¹ and R²² taken together with the carbon atoms to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino;
   wherein said polycarbonate or copolymer thereof is characterized by a ¹³C{¹H} NMR spectrum comprising only one signal present in the areas ranging from δ 150 to 155 ppm.

Preferably, the present invention also encompasses a polycarbonate or copolymer thereof comprising recurring units of formula (VII3) or of formula (VII4),

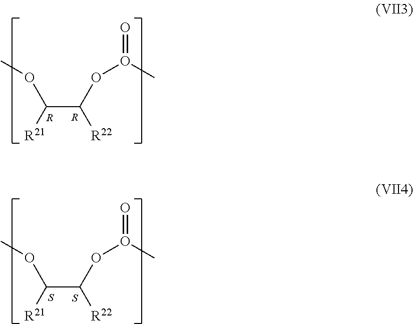

wherein
R²¹ and R²² taken together with the carbon atoms 4 and 5 to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; and
   wherein said polycarbonate or copolymer thereof is characterized by a ¹³C{¹H} NMR spectrum comprising only one signal present in the carbonyl areas ranging from δ 150 to 155 ppm; preferably said polycarbonate or copolymer thereof is characterized by a high resolution (100 MHz or higher)¹³C{¹H} NMR spectrum comprising only one sharp resonance present in the carbonyl areas ranging from δ 150 to 155 ppm.

In an embodiment, said polycarbonate or copolymer thereof according to the fourth aspect of the invention also comprises less than 2% of monomer having at least one chiral center of inverse configuration compared to that of the enantiopure monomer. This percentage can be measured, after hydrolysis of the precipitated polymer, by chiral chromatography of the vicinal diol resulting from the hydrolysis.

In a fifth aspect, the present invention also encompasses an article comprising a polycarbonate or copolymer thereof according to the third or fourth aspect of the invention, or a polycarbonate prepared according to the first aspect of the invention.

The processes of the present invention allow the production of polycarbonates and copolymers thereof with controlled tacticity, and improved properties such as high molecular mass and narrow molecular mass distribution, and original thermal features, in an efficient and economical way. In addition, the processes of the present invention allow the preparation of polycarbonates and copolymers thereof in a one-pot and one-step method.

The independent and dependent claims set out particular and preferred features of the invention. Features from the dependent claims may be combined with features of the independent or other dependent claims as appropriate.

The above and other characteristics, features and advantages of the present invention will become apparent from the following detailed descriptions, taken in conjunction with the accompanying drawings, which illustrate, by way of examples, the principles of the invention.

The reference figures quoted below refer to the attached drawings.

### BRIEF DESCRIPTION OF THE DRAWINGS

FIG. 1 represents the formula as well as the ¹H NMR spectrum (400 MHz, CDCl₃, 25° C.) of a PCHC synthesized from trans-rac-CHC in the presence of [(NNO)ZnEt]/BnOH in toluene at 100° C. (Example 1, Table 1, entry 8; CDCl₃).

FIG. 2 represents the formula as well as the ¹³C{¹H} NMR spectrum (100 MHz, CDCl₃, 25° C.) of a PCHC synthesized from trans-rac-CHC in the presence of [(NNO)ZnEt]/BnOH in toluene at 100° C. (PCHC of Example 1, Table 1, entry 5).

FIG. 3 represents a graph plotting the differential scanning calorimetry (DSC) trace of a PCHC sample synthesized from trans-rac-CHC (PCHC of Example 1, Table 1, entry 3).

FIG. 4 represents the ¹H NMR spectrum (400 MHz, CDCl₃, 25° C.) of a copolymer PLLA-co-PCHC synthesized from trans-rac-CHC and L-LA in the presence of a [(NNO)ZnEt]/BnOH system in toluene at 100° C. (with presence of residual CH₂Cl₂ and MeOH following precipitation of the polymer).

FIG. 5 represents a graph plotting the DSC trace of a sample of the copolymer PLLA-co-PCHC with a Mn of 15,760 g/mol.

FIG. 6 represents the formula as well as the ¹³C{¹H} NMR spectrum (100 MHz, CDCl₃, 25° C.) of a PCHC synthesized from (R,R)—CHC in the presence of [(NNO)ZnEt]/BnOH in toluene at 60° C. (PCHC of Example 2, Table 2, entry 1)

FIG. 7 represents a graph plotting the differential scanning calorimetry (DSC) trace of a PCHC sample (PCHC of Example 2, Table 2, entry 2).

FIG. 8 section a) represents part of a ¹³C{¹H} NMR spectrum (100 MHz, CDCl₃, 25° C.) of a PCHC synthesized by ROP of rac-CHC according to an embodiment of the invention (Table 4, entry 1′); section b) represents part of ¹³C{¹H} NMR spectrum (100 MHz, CDCl₃, 25° C.) of a PCHC synthesized by ROP of rac-CHC, according to an embodiment of the invention (Table 4, entries 5 and 6, the latter corresponding to the darker line).

FIG. 9 represents the formula as well as the ¹H NMR spectrum (500 MHz, CD₂Cl₂, 25° C.) of a copolymer PLLA-co-PCHC according to an embodiment of the invention (Table 5, entry 8).

FIG. 10 represents the formula as well as the ¹³C{¹H} NMR spectrum (100 MHz, CD₂Cl₂, 25° C.) of a copolymer PLLA-co-PCHC according to an embodiment of the invention (Table 5, entry 8); wherein x stands for unreacted CHC, and * stands for residual solvent resonances.

FIG. 11 represents the formula as well as the ¹H NMR spectrum (500 MHz, CDCl₃, 25° C.) of a PCHC-b-PLLA copolymer according to an embodiment of the invention (Table 6, entry 2).

FIG. 12 represents the formula as well as the ¹³C{¹H} NMR spectrum (100 MHz, CDCl₃, 25° C.) of a PCHC-b-PLLA copolymer according to an embodiment of the invention (Table 6, entry 2), wherein * stands for residual solvent resonances.

FIG. 13 represents the formula as well as the ¹H NMR spectrum (400 MHz, CDCl₃, 25° C.) of the crude product from the sequential copolymerization rac-CHC:LLA (1:1) according to an embodiment of the invention (Table 6, entry 2′).

FIG. 14 represents the formula as well as the ¹H NMR spectrum (400 MHz, CDCl₃, 25° C.) of the PCHC-b-PLLA copolymer obtained from precipitation of the crude reaction mixture in Example 8 (produced from the sequential copolymerization rac-CHC:LLA (1:1) described in Table 6, entry 2′) in cold methanol.

FIG. 15 represents the formula as well as the ¹H NMR spectrum (400 MHz, CDCl₃, 25° C.) of the PLLA-b-PCHC copolymer described in Example 8 (obtained by sequential copolymerization of L-LA and rac-CHC, where the L-LA was added before the addition of rac-CHC), wherein * stands for residual solvent resonances.

FIG. 16 represents the formula as well as the ¹³C{¹H} NMR spectrum (100 MHz, CD₂Cl₂, 25° C.) of the PLLA-b-PCHC copolymer described in Example 8 (obtained by sequential copolymerization of L-LA and rac-CHC, where the L-LA was added before the addition of rac-CHC), wherein * stands for residual solvent resonances.

FIG. 17 represents the formula as well as the ¹H NMR spectrum (400 MHz, CD₂Cl₂, 25° C.) of a PCHC-b-PLLA copolymer according to an embodiment of the invention (Table 8, entry 2), wherein * stands for residual solvent resonances.

FIG. 18 represents the formula as well as the ¹³C{¹H} NMR spectrum (100 MHz, CD₂Cl₂, 25° C.) of a PLLA-b-PCHC copolymer according to an embodiment of the invention (Table 8, entry 2), wherein * stands for residual solvent resonances.

FIG. 19 represents the formula as well as the ¹H NMR spectrum (500 MHz, CDCl₃, 25° C.) of a PCHC-co-PTMC copolymer according to an embodiment of the invention (Table 9, entry 2), wherein * stands for residual solvent resonances.

FIG. 20 represents the formula as well as the ¹³C{¹H} NMR spectrum (100 MHz, CDCl₃, 25° C.) of a PCHC-co-PTMC copolymer according to an embodiment of the invention (Table 9, entry 2), wherein * stands for residual solvent resonances.

FIG. 21 represents the formula as well as the ¹³C{¹H} NMR spectrum (100 MHz, CDCl₃, 25° C.) of a PCHC-co-PTMC copolymer according to an embodiment of the invention (Table 10, entry 2), wherein * stands for residual solvent resonances.

FIG. 22 represents the formula as well as the ¹H NMR spectrum (500 MHz, CDCl₃, 25° C.) of a PCHC-co-PTMC copolymer according to an embodiment of the invention (Table 11, entry 2), wherein * stands for residual solvent resonances.

FIG. 23 represents the formula as well as the ¹³C{¹H} NMR spectrum (100 MHz, CDCl₃, 25° C.) of a PCHC-co-PTMC copolymer according to an embodiment of the invention (Table 11, entry 2), wherein * stands for residual solvent resonances.

### DETAILED DESCRIPTION OF THE INVENTION

Before the present process of the invention is described, it is to be understood that this invention is not limited to particular processes, components, or devices described, as such processes, components, and devices may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

When describing the present invention, the terms used are to be construed in accordance with the following definitions, unless a context dictates otherwise.

Whenever the term “substituted” is used in the present invention, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture.

The term “C₁₋₂₀alkyl”, as a group or part of a group, refers to a hydrocarbyl radical of Formula CₙH₂ₙ₊₁ wherein n is a number ranging from 1 to 20. Generally, the alkyl groups comprise from 1 to 20 carbon atoms, preferably from 1 to 12 carbon atoms, preferably from 1 to 10 carbon atoms, preferably from 1 to 6 carbon atoms, more preferably 1, 2, 3, 4, 5, 6 carbon atoms. Alkyl groups may be linear, or branched and may be substituted as indicated herein. When a subscript is used herein following a carbon atom, the subscript refers to the number of carbon atoms that the named group may contain. Thus, for example, C₁₋₂₀alkyl groups include all linear, or branched alkyl groups having 1 to 20 carbon atoms, and thus includes for example methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers, nonyl and its isomers, decyl and its isomers, undecyl and its isomers, dodecyl and its isomers, tridecyl and its isomers, tetradecyl and its isomers, pentadecyl and its isomers, hexadecyl and its isomers, heptadecyl and its isomers, octadecyl and its isomers, nonadecyl and its isomers, icosyl and its isomers, and the like. For example, C₁₋₁₀alkyl includes all linear, or branched alkyl groups having 1 to 10 carbon atoms, and thus includes for example methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers, nonyl and its isomers, decyl and its isomers and the like. For example, C₁₋₆alkyl includes all linear, or branched alkyl groups having 1 to 6 carbon atoms, and thus includes for example methyl, ethyl, n-propyl, i-propyl, 2-methyl-ethyl, butyl and its isomers (e.g. n-butyl, i-butyl and t-butyl); pentyl and its isomers, hexyl and its isomers. When the suffix “ene” is used in conjunction with an alkyl group, i.e. “alkylene”, this is intended to mean the alkyl group as defined herein having two single bonds as points of attachment to other groups.

The term “C₁₋₆alkoxy” or “C₁₋₆alkyloxy”, as a group or part of a group, refers to a group having the Formula —OR^{a} wherein R^{a} is C₁₋₆alkyl. Non-limiting examples of suitable C₁₋₆alkoxy include methoxy, ethoxy, propoxy, isopropoxy, butoxy, isobutoxy, sec-butoxy, tert-butoxy, pentyloxy and hexyloxy.

The term “C₃₋₉cycloalkyl”, as a group or part of a group, refers to a cyclic alkyl group, that is to say, a monovalent, saturated, hydrocarbyl group having 1 or more cyclic structure, and comprising from 3 to 9 carbon atoms, more preferably from 3 to 8 carbon atoms, more preferably from 3 to 6 carbon atoms, still more preferably from 5 to 6 carbon atoms. Cycloalkyl includes all saturated hydrocarbon groups containing 1 or more rings, including monocyclic or bicyclic groups. The further rings of multi-ring cycloalkyls may be either fused, bridged and/or joined through one or more spiro atoms. Examples of C₃₋₆cycloalkyl groups include but are not limited to cyclopropyl, cyclobutyl, cyclopentyl, cyclohexyl. When the suffix “ene” is used in conjunction with a cycloalkyl group, i.e. cycloalkylene, this is intended to mean the cycloalkyl group as defined herein having two single bonds as points of attachment to other groups.

The term “C₅₋₉cycloalkenyl”, as a group or part of a group, refers to a group derived from a cycloalkyl group, as defined above, comprising from 5 to 9 carbon atoms, and comprising one or more double bonds. For example, this could be the group cyclopentenyl, cyclohexenyl, cyclopenta-1,3-dienyl, cycloheptenyl, cyclooctenyl, cycloocta-1,4-dienyl. When the suffix “ene” is used in conjunction with a cycloalkenyl group, i.e. cycloalkenylene, this is intended to mean the cycloalkenyl group as defined herein having two single bonds as points of attachment to other groups.

The term “C₆₋₃₀aryl”, as a group or part of a group, refers to a polyunsaturated, aromatic hydrocarbyl group having a single ring (i.e. phenyl) or multiple aromatic rings fused together (e.g. naphthalene), or linked covalently, typically containing 6 to 30 atoms; wherein at least one ring is aromatic. Examples of suitable aryl include C₆₋₁₂aryl, more preferably C₆₋₁₀aryl. Non-limiting examples of C₆₋₁₂aryl comprise phenyl, biphenylyl, biphenylenyl, or 1-or 2-naphthanelyl. When the suffix “ene” is used in conjunction with an aryl group, this is intended to mean the aryl group as defined herein having two single bonds as points of attachment to other groups.

The term “C₆₋₃₀arylC₁₋₂₀alkyl”, as a group or part of a group, means a C₁₋₂₀alkyl as defined herein, wherein at least one hydrogen atom is replaced by at least one C₆₋₃₀aryl as defined herein. Preferred C₆₋₃₀arylC₁₋₂₀alkyl includes C₆₋₁₂arylC₁₋₆alkyl. Non-limiting examples of C₆₋₁₂arylC₁₋₆alkyl group include benzyl, phenethyl, dibenzylmethyl, methylphenylmethyl, 3-(2-naphthyl)-butyl, and the like.

The term “halo” or “halogen”, as a group or part of a group, is generic for fluoro, chloro, bromo or iodo.

The term “haloC₁₋₆alkyl”, as a group or part of a group, refers to a C₁₋₆alkyl group having the meaning as defined above wherein one or more hydrogens are replaced with a halogen as defined above. Non-limiting examples of such haloC₁₋₆alkyl radicals include chloromethyl, 1-bromoethyl, fluoromethyl, difluoromethyl, trifluoromethyl, 1,1,1-trifluoroethyl and the like.

The term “haloC₁₋₆alkoxy”, as a group or part of a group, refers to a group of Formula —O—R^{b} wherein R^{b} is haloC₁₋₆alkyl as defined herein. Non-limiting examples of suitable haloC₁₋₆alkoxy include fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy, 2,2,2-trichloroethoxy, trichloromethoxy, 2-bromoethoxy, pentafluoroethyl, 3,3,3-trichloropropoxy, 4,4,4-trichlorobutoxy.

The term “hydroxyl” or “hydroxy”, as a group or part of a group, refers to the group —OH.

The term “C₁₋₆alkylthio”, as a group or part of a group, refers to a group having the Formula —SR^{a} wherein R^{a} is C₁₋₆alkyl. Non-limiting examples of C₁₋₆alkylthio groups include methylthio (—SCH₃), ethylthio (—SCH₂CH₃), n-propylthio, isopropylthio, n-butylthio, isobutylthio, sec-butylthio, tert-butylthio, and the like.

The term “amino” refers to the group —NH₂.

The term “mono- or di-C₁₋₆alkylamino”, as a group or part of a group, refers to a group of formula —N(R^{c})(R^{d}) wherein R^{c} and R^{d} are each independently selected from hydrogen, or C₁₋₆alkyl, wherein at least one of R^{c} or R^{d} is C₁₋₆alkyl. Thus, alkylamino include mono-alkyl amino group (e.g. mono-C₁₋₆alkylamino group such as methylamino and ethylamino), and di-alkylamino group (e.g. di-C₁₋₆alkylamino group such as dimethylamino and diethylamino). Non-limiting examples of suitable alkylamino groups include n-propylamino, isopropylamino, n-butylamino, i-butylamino, sec-butylamino, t-butylamino, pentylamino, n-hexylamino, di-n-propylamino, di-i-propylamino, ethylmethylamino, methyl-n-propylamino, methyl-i-propylamino, n-butylmethylamino, i-butylmethylamino, t-butylmethylamino, ethyl-n-propylamino, ethyl-i-propylamino, n-butylethylamino, i-butylethylamino, t-butylethylamino, di-n-butylamino, di-i-butylamino, methylpentylamino, methylhexylamino, ethylpentylamino, ethylhexylamino, propylpentylamino, propylhexylamino, and the like.

The term “carboxy” or “carboxyl”, as a group or part of a group, refers to the group —CO₂H.

The term “C₁₋₆alkoxycarbonyl”, as a group or part of a group, refers to a carboxy group linked to a C₁₋₆alkyl radical i.e. to form —C(═O)OR^{a}, wherein R^{a} is as defined above for C₁₋₆alkyl.

The term “C₁₋₆alkylcarbonyloxy”, as a group or part of a group, refers to a group of Formula —O—C(═O)R^{a} wherein R^{a} is as defined above for C₁₋₆alkyl.

The term “heterocyclyl”, as a group or part of a group, refers to a saturated, unsaturated or aromatic ring system of 4 to 18 atoms including at least one N, O, S, or P. Heterocyclyl thus include heteroaryl groups. Heterocyclyl as used herein includes by way of example and not limitation the heterocycles described in Paquette, Leo A. “Principles of Modern Heterocyclic Chemistry” (W.A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; Katritzky, Alan R., Rees, C. W. and Scriven, E. “Comprehensive Heterocyclic Chemistry” (Pergamon Press, 1996); and J. Am. Chem. Soc. (1960) 82:5566. In a preferred embodiment, heterocyclyl is selected from the group comprising saturated, unsaturated and aromatic ring systems comprising 4 to 10 atoms including at least one N, O, or S. In a preferred embodiment, heterocyclyl is selected from the group comprising saturated, unsaturated and aromatic ring systems comprising 5 to 9 atoms including at least one N, O, or S. In a preferred embodiment, heterocyclyl is selected from the group comprising saturated, unsaturated and aromatic ring systems comprising 5 to 9 atoms including at least one N, or O. Heterocyclyl thus include heteroaryl groups. In a particular embodiment, heterocyclyl is selected from the group comprising 1,3-dioxanyl, 1,4-dioxanyl; pyridyl, dihydroypyridyl, tetrahydropyridyl (piperidyl), thiazolyl, tetrahydrothiophenyl, sulfur oxidized tetrahydrothiophenyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, benzothienyl, benzothiazolyl and isatinoyl; preferably heterocyclyl is selected from the group comprising 1,3-dioxanyl, 1,4-dioxanyl; pyridyl, dihydroypyridyl, tetrahydropyridyl (piperidyl), thiazolyl, tetrahydrothiophenyl, furanyl, thienyl, pyrrolyl, pyrazolyl, imidazolyl, tetrazolyl, benzofuranyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, bis-tetrahydrofuranyl, tetrahydropyranyl, bis-tetrahydropyranyl, tetrahydroquinolinyl, tetrahydroisoquinolinyl, decahydroquinolinyl, octahydroisoquinolinyl, azocinyl, triazinyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, pyrimidinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, benzothienyl, benzothiazolyl and isatinoyl. The term “heteroaryl”, as a group or part of a group, refers to an aromatic ring system of 5 to 18 atoms including at least one N, O, S, or P and thus refers to aromatic heterocycles. Examples of heteroaryl include but are not limited to pyridyl, pyridazinyl, pyrimidinyl, pyrazinyl, s-triazinyl, oxazolyl, imidazolyl, thiazolyl, isoxazolyl, pyrazolyl, isothiazolyl, furyl, thienyl, and pyrrolyl. The term “non-aromatic heterocyclyl”, as a group or part of a group, refers to a saturated or unsaturated non-aromatic ring system of 4 to 18 atoms including at least one N, O, S, or P.

Preferred statements (features) and embodiments of the processes and polycarbonates of this invention are set herein below. Each statements and embodiments of the invention so defined may be combined with any other statement and/or embodiments unless clearly indicated to the contrary. In particular, any feature indicated as being preferred or advantageous may be combined with any other feature or features indicated as being preferred or advantageous. Numbered statements of this invention are:
1. A process for preparing a polycarbonate or copolymer thereof, said process comprising homo- or copolymerizing in the presence of at least one catalyst, one or more compounds of formula (I), stereoisomers, racemics, or mixtures thereof; optionally with one or more cyclic esters, stereoisomers, racemics, or mixtures thereof;

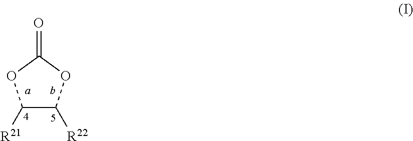

wherein
each dotted bond “a” and “b” independently represents a solid bond, a wedged bond, or a hashed wedged bond;
each R²¹ and R²² are, independently, selected from C₁₋₆alkyl, C₃₋₆cycloalkyl, and C₆₋₁₀aryl; or R²¹ and R²² taken together with the carbon atoms 4 and 5 of the cyclic carbonate form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.
2. The process according to claim 1, wherein the catalyst is an organometallic catalyst of formula (II) or (III),

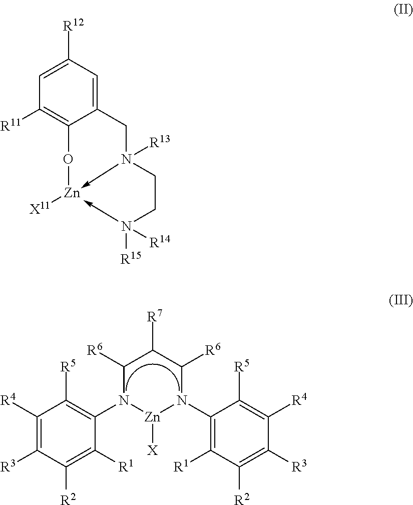

wherein
R¹, R², R³, R⁴, R⁵, R⁶, and R⁷ are each independently selected from the group consisting of hydrogen, optionally substituted C₁₋₁₂alkyl, and an inert functional group, and wherein two or more of said groups can be linked together to form one or more rings;
X is —N(SiR²⁷₃)₂, C₁₋₁₂alkyl, C₁₋₁₂alkoxy, —NR⁹R¹⁰ or —BH₄;
each R²⁷ is independently selected from hydrogen and C₁₋₆alkyl;
each R⁹ and R¹⁰ is independently selected from hydrogen and C₁₋₆alkyl;
R¹¹ and R¹² are each independently C₁₋₁₀alkyl;
R¹³, R¹⁴, and R¹⁵ are each independently C₁₋₁₀alkyl, or
R¹³ and R¹⁴ are covalently bound to each other and are each a methylene and R¹⁵ is C₁₋₁₀alkyl;
X¹¹ is selected from C₁₋₁₀alkyl, —OR¹⁶, and —N(SiR¹⁷₃)₂;
R¹⁶ is C₁₋₁₀alkyl; and
each R¹⁷ is independently selected from hydrogen and C₁₋₆alkyl.
3. The process according to any one of statements 1 to 2, wherein the catalyst is selected from [(NNO)ZnEt], [BDI]Zn(N(SiMe₃)₂), [BDI]Zn(Et), and {[BDI]Zn(OR³⁰)}₂, wherein R³⁰ is C₁₋₆alkyl; preferably the catalyst is [(NNO)ZnEt].

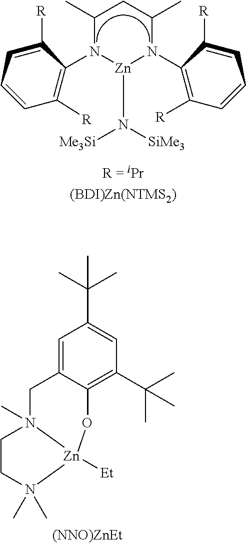

4. The process according to statement 1, wherein the catalyst is selected from complexes of formulae M(OSO₂CF₃)ₘ, M(N(OSO₂CF₃)₂)ₘ, M(R²³C(O)CR²³₂C(O)R²³)ₘ, and (R²⁴CO₂)ₘM, wherein M is a metal of Group 2, 3, including the lanthanide series, or Group 12, 13, 15, wherein each R²³ is independently an optionally substituted C₁₋₁₂alkyl, wherein each R²⁴ is independently a perfluorinated C₁₋₁₂alkyl or an aryl, and wherein m is the valence of M.
5. The process according to any one of statements 1 to 4, wherein the process is performed in the presence of a compound of formula (IV),

R⁸—OH (IV)
wherein R⁸ is selected from the group consisting of C₁₋₂₀alkyl, C₆₋₃₀aryl, and C₆₋₃₀arylC₁₋₂₀alkyl optionally substituted by one or more substituents selected from the group consisting of halogen, hydroxyl, and C₁₋₆alkyl.
6. The process according to any one of statements 1 to 5, wherein the cyclic ester is selected from the group comprising lactide, glycolide, β-butyrolactone, δ-valerolactone, γ-butyrolactone, γ-valerolactone, 4-methyldihydro-2(3H)-furanone, alpha-methyl-gamma-butyrolactone, and ε-caprolactone, and mixture thereof.
7. The process according to any one of statements 1 to 6, wherein the process is performed in the presence or absence (in bulk) of solvent.
8. The process according to statement 7, wherein the solvent is selected from an aliphatic or aromatic hydrocarbon, an ether, and an halogenated solvent.
9. A process for the synthesis of a compound of formula (I) a stereoisomer or racemate thereof, comprising the step of contacting a compound of formula (V), a stereoisomer, or racemate thereof; with at least one reagent selected from the group consisting of an alkyl- or cycloalkyl-chloroformate of formula (VI); an urea; a phosgene or derivative thereof; a cyclic carbonate; and carbon dioxide;

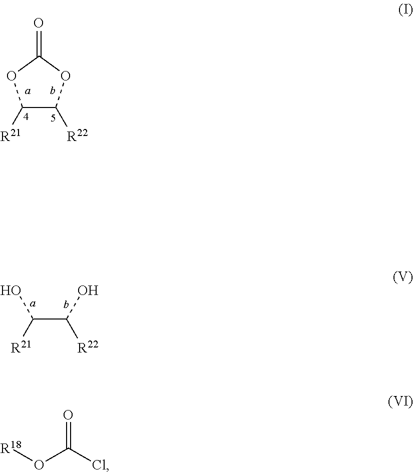

wherein
each dotted bond “a” and “b” independently represents a solid bond, a wedged bond, or a hashed wedged bond;
each R²¹ and R²² are, independently, selected from C₁₋₆alkyl, C₃₋₆cycloalkyl, and C₆₋₁₀aryl; or R²¹ and R²² taken together with the carbon atoms to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; and
R¹⁸ is C₁₋₆alkyl, or C₃₋₆cycloalkyl.
10. A polycarbonate or copolymer thereof comprising a recurring unit of formula (VII1) and/or (VII2),

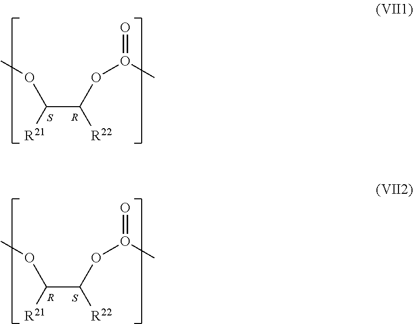

wherein
each R²¹ and R²² are, independently, selected from C₁₋₆alkyl, C₃₋₆cycloalkyl, and C₆₋₁₀aryl; or R²¹ and R²² taken together with the carbon atoms to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.
11. The polycarbonate or copolymer thereof according to statement 10, comprising recurring units each independently represented by the following formula (VIIA) or (VIIB) or (VIIC);

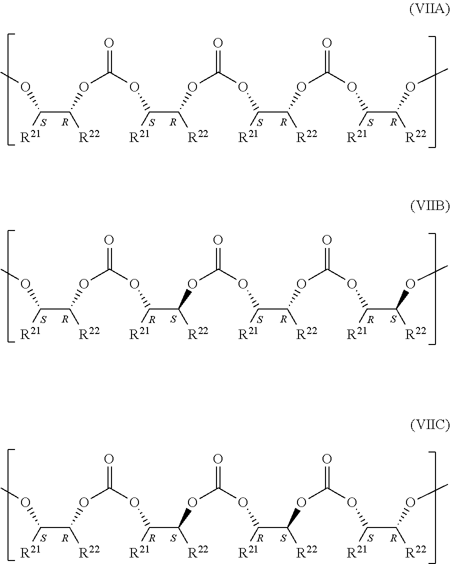

wherein R²¹ and R²² have the same meaning as that defined in statement 10.
12. The polycarbonate or copolymer thereof according to statement 10 or 11, comprising recurring units each independently represented by the following formula (VIIA1) or (VIIB1) or (VIIC1);

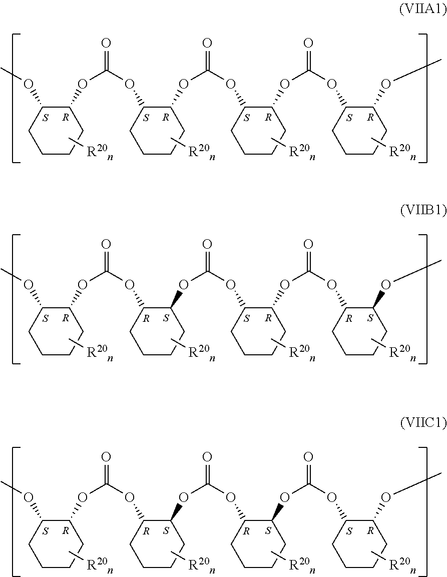

each R²⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino;
n is an integer selected from 0, 1, 2, 3, or 4.
13. The polycarbonate or copolymer thereof according to statement 10 or 11, comprising recurring units each independently represented by the following formula (VIIA2) or (VIIB2) or (VIIC2);

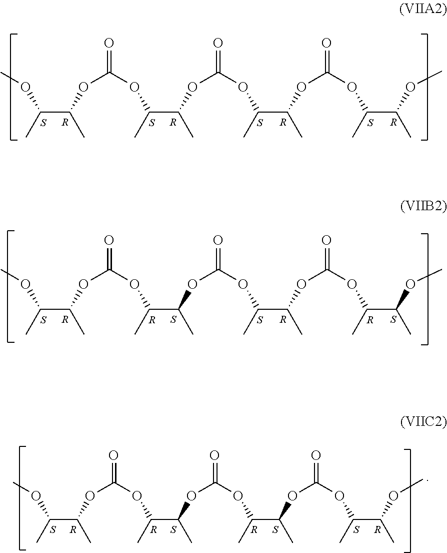

14. A polycarbonate or copolymer thereof comprising recurring units of formula (VII3) or of formula (VII4),
   wherein

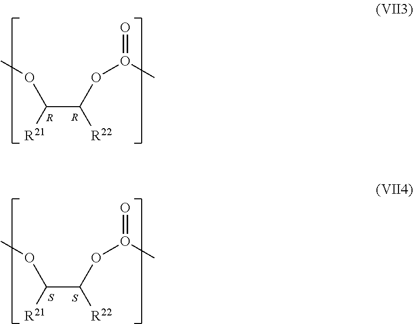

each R²¹ and R²² are, independently, selected from C₁₋₆alkyl, C₃₋₆cycloalkyl, and C₆₋₁₀aryl; or R²¹ and R²² taken together with the carbon atoms 4 and 5 to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆-12aryl, C₁₋₆alkylthio, carboxyl, C₁₋₆alkoxycarbonyl, C₁₋₆alkylcarbonyloxy, amino; or mono- or di-C₁₋₆alkylamino; and
wherein said polycarbonate or copolymer thereof is characterized by a ¹³C{¹H} NMR spectrum comprising only one signal present in the areas ranging from δ 150 to 155 ppm.
15. An article comprising a polycarbonate or copolymer thereof according to any one of statements 10 to 14 or a polycarbonate or copolymer thereof prepared according to a process according to any of statements 1 to 8.
16. A process for preparing a polycarbonate or copolymer thereof, said process comprising homo- or copolymerizing in the presence of at least one catalyst, one or more compounds of formula (I), stereoisomers, racemics, or mixtures thereof; optionally with one or more cyclic esters, stereoisomers, racemics, or mixtures thereof;

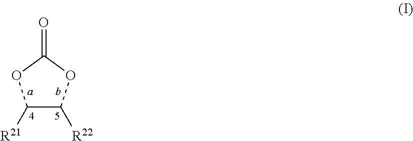

wherein
each dotted bond “a” and “b” independently represents a solid bond, a wedged bond, or a hashed wedged bond; and
R²¹ and R²² taken together with the carbon atoms 4 and 5 of the cyclic carbonate form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.
17. A process for preparing a polycarbonate or copolymer thereof, said process comprising copolymerizing in the presence of at least one catalyst, one or more compounds of formula (I), stereoisomers, racemics, or mixtures thereof; with one or more cyclic esters, stereoisomers, racemics, or mixtures thereof;

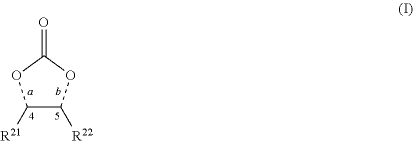

wherein
each dotted bond “a” and “b” independently represents a solid bond, a wedged bond, or a hashed wedged bond; and
R²¹ and R²² taken together with the carbon atoms 4 and 5 of the cyclic carbonate form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; wherein two of said one or more substituents can join to form a ring fused to said C₃₋₉cycloalkylene, C₅₋₉cycloalkenylene, or C₆₋₁₂arylene; wherein said ring formed by said two substituents is selected from the group comprising C₃₋₉cycloalkyl, C₅₋₉cycloalkenyl, C₆₋₁₂aryl, and heterocyclyl, wherein each group can be unsubstituted or substituted with one or more R⁴⁰ wherein each R⁴⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.
18. The process according to any one of statements 1-3, 5-8, 16-17, wherein the catalyst is an organometallic catalyst of formula (II) or (III),

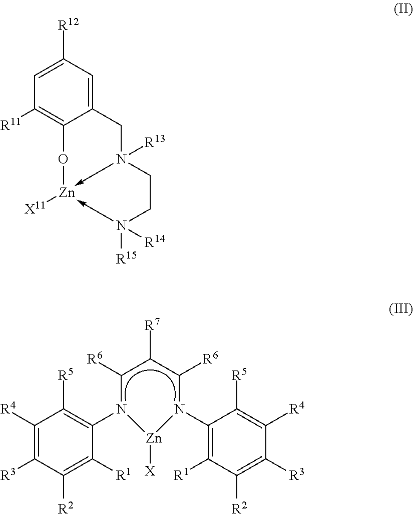

wherein
R¹, R², R³, R⁴, R⁵, R⁶, and R⁷ are each independently selected from the group consisting of hydrogen, optionally substituted C₁₋₁₂alkyl, and an inert functional group, and wherein two or more of said groups can be linked together to form one or more rings;
X is —N(SiR²⁷₃)₂, C₁₋₁₂alkyl, C₁₋₁₂alkoxy, —NR⁹R¹⁰ or —BH₄;
each R²⁷ is independently selected from hydrogen and C₁₋₆alkyl;
each R⁹ and R¹⁰ is independently selected from hydrogen and C₁₋₆alkyl;
R¹¹ and R¹² are each independently C₁₋₁₀alkyl;
R¹³, R¹⁴, and R¹⁵ are each independently C₁₋₁₀alkyl, or
R¹³ and R¹⁴ are covalently bound to each other and are each a methylene and R¹⁵ is C₁₋₁₀alkyl;
X¹¹ is selected from C₁₋₁₀alkyl, —OR¹⁶, and —N(SiR¹⁷₃)₂;
R¹⁶ is C₁₋₁₀alkyl; and
each R¹⁷ is independently selected from hydrogen and C₁₋₆alkyl.
19. The process according to any one of statements 1-3, 5-8, 16-18, wherein the catalyst is selected from [(NNO)ZnEt], [BDI]Zn(N(SiMe₃)₂), [BDI]Zn(Et), and {[BDI]Zn(OR³⁰)}₂, wherein R³⁰ is C₁₋₆alkyl,

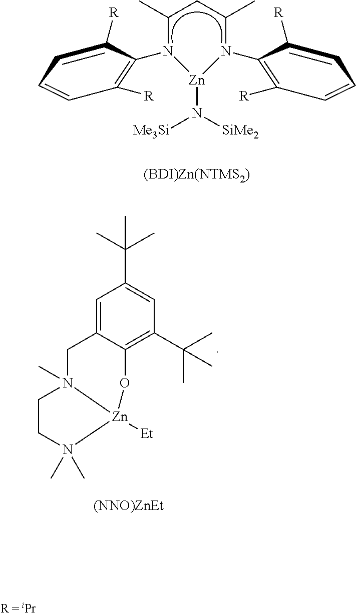

20. The process according to any one of statements 1-3, 5-8, 16-19, wherein the catalyst is [(NNO)ZnEt].
21. The process according to any one of statements 1, 4-8, 16-17, wherein the catalyst is selected from complexes of formulae M(OSO₂CF₃)ₘ, M(N(OSO₂CF₃)₂)ₘ, M(R²³C(O)CR²³₂C(O)R²³)ₘ, and (R²⁴CO₂)ₘM, wherein M is a metal of Group 2, 3, including the lanthanide series, or Group 12, 13, 15, wherein each R²³ is independently an optionally substituted C₁₋₁₂alkyl, wherein each R²⁴ is independently a perfluorinated C₁₋₁₂alkyl or an aryl, and wherein m is the valence of M.
22. The process according to any one of statements 1, 5-8, 16-17, wherein the catalyst is selected from the group comprising dimeric phosphazene bases, or amines or guanidines, organic acids, sparteine, thiourea-amino derivatives, N-heterocyclic carbenes, 4-(dialkylamino)pyridines, amidines, phosphorus based phosphines, phosphazenium derivatives and phosphazenes, metal amides; and compounds of general formula M¹(Y¹, Y², . . . Y^{P})_{q}, in which M¹ is a metal selected from the group comprising the elements of columns 3 to 12 of the periodic table of the elements, and the elements Al, Ga, In, Tl, Ge, Sn, Pb, Sb, Ca, Mg and Bi; whereas Y¹, Y², . . . Y^{p} are each substituents selected from the group comprising alkyl with 1 to 20 carbon atoms, aryl having from 6 to 30 carbon atoms, alkoxy having from 1 to 20 carbon atoms, aryloxy having from 6 to 30 carbon atoms, oxide, carboxylate, and halide groups, and p and q are integers of from 1 to 6.
23. The process according to any one of statements 1, 5-8, 16-17, 22, wherein the catalyst is selected from the group comprising 1,5,7-triazobicyclo-[4,4,0]dec-5-ene (TBD), 1,8-diazabicyclo[5.4.0]undec-7-ene (DBU), 4-dimethylaminopyridine (DMAP), and tert-butylimino-1,3-dimethylperhydro-1,3,2-diazaphosphine (BEMP); Sn(Oct)₂, Al(OiPr)₃, Ti(OiPr)₄, Ti(2-ethylhexanoate)₄, Ti(2-ethylhexyloxide)₄, Zr(OiPr)₄, Bi(neodecanoate)₃, (2,4-di-tert-butyl-6-(((2-(dimethylamino)ethyl)(methyl)amino)methyl)phenoxy)(ethoxy)zinc, Zn(lactate)₂, Y[N(SiMe₃)₂]₃; [Y]+L1; and [Y]+L2, wherein L1 is a tetradentate ligand of formula L1 and L2 is a tetradentate ligand of formula L2

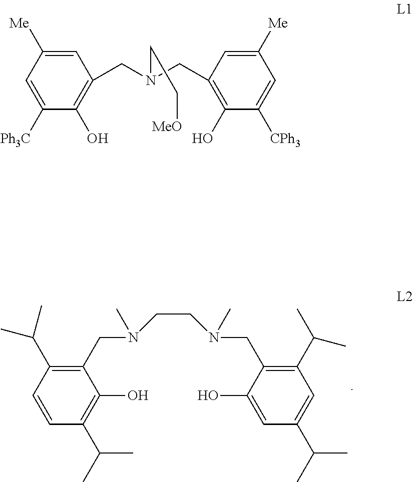

24. The process according to any one of statements 1, 5-8, 16-17, wherein the catalyst is selected from the group comprising [(NNO)ZnEt]; [BDI]Zn(N(SiMe₃)₂); 1,5,7-triazobicyclo-[4,4,0]dec-5-ene (TBD); 1,8-diazabicyclo[5.4.0]undec-7-ene (DBU); Y[N(SiMe₃)₂]₃; [Y]+L1; and [Y]+L2; wherein L1 is a tetradentate ligand of formula L1 and L2 is a tetradentate ligand of formula L2

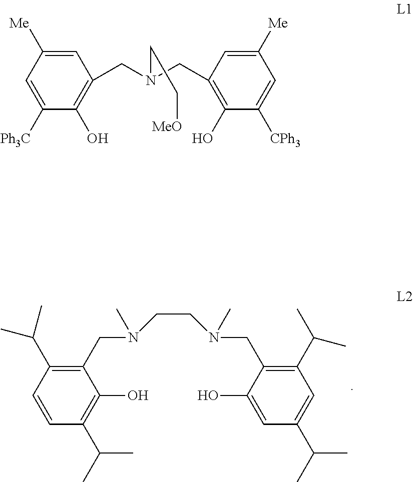

25. The process according to any one of statements 1-8, 16-24, wherein the process is performed in the presence of a compound of formula (IV),

R⁸—OH (IV)
wherein R⁸ is selected from the group consisting of C₁₋₂₀alkyl, C₆₋₃₀aryl, and C₆₋₃₀arylC₁₋₂₀alkyl optionally substituted by one or more substituents selected from the group consisting of halogen, hydroxyl, and C₁₋₆alkyl.
26. The process according to any one of statements 1-5, 7-8, 16-25, wherein the cyclic ester is selected from the group comprising lactones, cyclic carbonates, glycolides and lactides, each group can be unsubstituted or substituted with one or more substituents each independently selected from C₁₋₆alkyl and C₁₋₆alkoxy.
27. The process according to any one of statements 1-5, 7-8, 16-26, wherein the cyclic ester is selected from the group comprising lactide, trimethylene carbonate, glycolide, β-butyrolactone, δ-valerolactone, γ-butyrolactone, γ-valerolactone, 4-methyldihydro-2(3H)-furanone, alpha-methyl-gamma-butyrolactone, and ε-caprolactone, 1,3-dioxolan-2-one, propylene carbonate, 4-methyl-1,3-dioxan-2-one, 1,3-doxepan-2-one, 5-C₁₋₄alkoxy-1,3-dioxan-2-one; and mixture thereof, wherein each group can be unsubstituted or substituted with one or more substituents each independently selected from C₁₋₆alkyl and C₁₋₆alkoxy. For example said cyclic ester can be selected from the group comprising lactide, trimethylene carbonate, glycolide, β-butyrolactone, δ-valerolactone, γ-butyrolactone, γ-valerolactone, 4-methyldihydro-2(3H)-furanone, alpha-methyl-gamma-butyrolactone, ε-caprolactone, and mixture thereof. For example said cyclic ester can be selected from the group comprising lactide, glycolide, β-butyrolactone, δ-valerolactone, γ-butyrolactone, γ-valerolactone, 4-methyldihydro-2(3H)-furanone, alpha-methyl-gamma-butyrolactone, ε-caprolactone, and mixture thereof. For example said cyclic ester is a lactide, such as L-lactide (S,S-lactide), D-lactide (R,R-lactide), or meso-(S,R)-lactide. For example, said cyclic ester is a cyclic carbonate, such as trimethylene carbonate.
28. The process according to any one of statements 1-7, 16-27, wherein the process is performed in the presence or absence (in bulk) of solvent.
29. The process according to any one of statements 1-7, 16-28, wherein the process is performed in the presence of solvent.
30. The process according to any one of statements 1-7, 16-28, wherein the process is performed in the presence of solvent selected from an aliphatic or aromatic hydrocarbon, an ether, an halogenated solvent and a C₁₋₆alkyl carbonate.
31. The process according to any one of statements 1-8, 16-30, wherein R²¹ and R²² taken together with the carbon atoms 4 and 5 of the cyclic carbonate form a ring selected from C₅₋₇cycloalkylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino, wherein two of said one or more substituents can join to form a ring fused to said C₅₋₇cycloalkylene, or C₆₋₁₀arylene ring.
32. The process according to any one of statements 1-8, 16-31, wherein R²¹ and R²² taken together with the carbon atoms 4 and 5 of the cyclic carbonate form a ring selected from C₆₋₇cycloalkylene, and C₆₋₁₀arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; wherein two of said one or more substituents can join to form a C₃₋₆cycloalkyl fused to said C₆₋₇cycloalkylene, or C₆₋₁₀arylene ring.
33. The process according to any one of statements 1-8, 16-33, wherein R²¹ and R²² taken together with the carbon atoms 4 and 5 of the cyclic carbonate form a cyclohexylene; optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino, for example one or more substituents each independently selected from halo, hydroxyl, C₁₋₆alkyl, C₁₋₆alkoxy, or C₁₋₆alkylthio; wherein two of said substituents can join to form a C₃₋₆cycloalkyl fused to said cyclohexylene ring.
34. The process according to any one of statements 1-8, 16-31, wherein R²¹ and R²² taken together with the carbon atoms 4 and 5 of the cyclic carbonate form a ring selected from C₃₋₉cycloalkylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.
35. The process according to any one of statements 1-8, 16-32, 34, wherein R²¹ and R²² taken together with the carbon atoms 4 and 5 of the cyclic carbonate form a ring selected from C₃₋₆cycloalkylene, and C₆₋₁₀arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.
36. The process according to any one of statements 1-8, 16-35, wherein R²¹ and R²² taken together with the carbon atoms 4 and 5 of the cyclic carbonate form C₃₋₆cycloalkylene, preferably a C₄₋₆cycloalkylene, preferably a C₅₋₆cycloalkylene, preferably a cyclohexylene; optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; for example one or more substituents each independently selected from halo, hydroxyl, C₁₋₆alkyl, C₁₋₆alkoxy, or C₁₋₆alkylthio.
37. The process according to any one of statements 1-8, 16-30, wherein the compound of formula (I) has structural formula (IA)

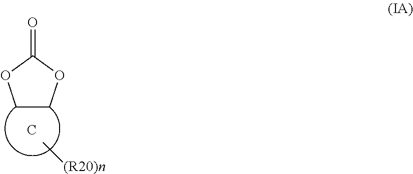

wherein
the ring C is selected from C₃₋₉cycloalkylene, C₅₋₉cycloalkenylene, or C₆₋₁₂arylene; preferably C₄₋₆cycloalkylene, or C₆₋₁₀arylene; more preferably C₅₋₆cycloalkylene;
each R²⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably halo, hydroxyl, C₁₋₁₀alkyl; C₁₋₁₀alkoxy, or C₁₋₆alkylthio; preferably C₁₋₆alkyl; more preferably C₁₋₄alkyl; optionally wherein two R²⁰ can join to form a ring fused to ring C, wherein said ring formed by two R²⁰ can be selected from the group comprising C₃₋₉cycloalkyl, C₅₋₉cycloalkenyl, C₆₋₁₂aryl, and heterocyclyl, preferably C₅₋₆cycloalkyl, or C₆₋₁₀aryl, and wherein said ring formed by two R²⁰ can be unsubstituted or substituted with one or more R⁴⁰, wherein each R⁴⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino;
n is an integer selected from 0, 1, 2, 3, or 4; preferably 0, 1, 2, or 3, preferably 0, 1, or 2, more preferably 0 or 1, yet more preferably 0.
38. The process according to any one of statements 1-8, 16-30, 37, wherein the compound of formula (I) has structural formula (IA1), (IA2), (IA3), or (IA4), wherein

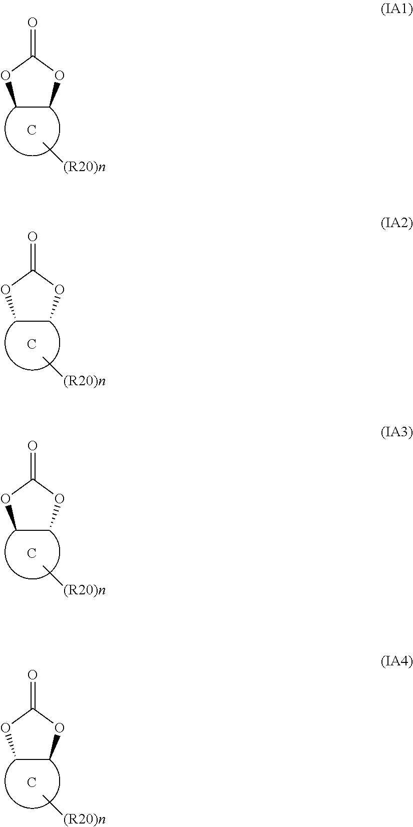

the ring C is selected from C₃₋₉cycloalkylene, C₅₋₉cycloalkenylene, or C₆₋₁₂arylene; preferably C₄₋₆cycloalkylene, or C₆₋₁₀arylene; more preferably C₅₋₆cycloalkylene; preferably hexylene, each R²⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably halo, hydroxyl, C₁₋₁₀alkyl; C₁₋₁₀alkoxy, or C₁₋₆alkylthio; preferably C₁₋₆alkyl; more preferably C₁₋₄alkyl; optionally wherein two R²⁰ can join to form a ring fused to ring C, wherein said ring formed by two R²⁰ can be selected from the group comprising C₃₋₉cycloalkyl, C₅₋₉cycloalkenyl, C₆₋₁₂aryl, and heterocyclyl, preferably C₅₋₆cycloalkyl, or C₆₋₁₀aryl, and wherein said ring formed by two R²⁰ can be unsubstituted or substituted with one or more R⁴⁰, wherein each R⁴⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably halo, hydroxyl, C₁₋₁₀alkyl; C₁₋₁₀alkoxy, or C₁₋₆alkylthio; preferably C₁₋₆alkyl; more preferably C₁₋₄alkyl;
n is an integer selected from 0, 1, 2, 3, or 4; preferably 0, 1, 2, or 3, preferably 0, 1, or 2, more preferably 0 or 1, yet more preferably 0.
39. The process according to any one of statements 1-8, 16-30, 37-38, wherein the compound of formula (I) is selected from the compounds of formula (IAa), (IA1a), (IA2a) (IA3a), (IA4a), (IC);

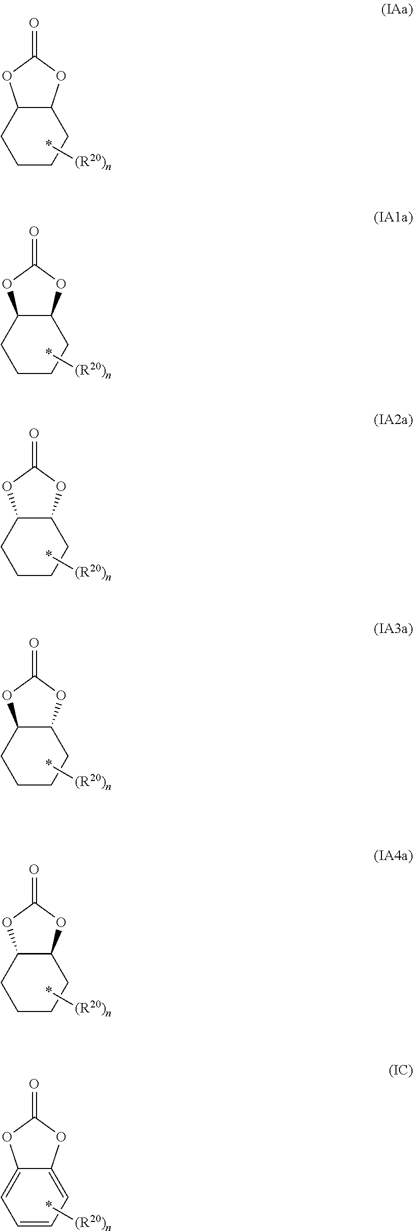

wherein R²⁰ is selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably halo, hydroxyl, C₁₋₆alkyl, haloC₁₋₆alkyl, C₁₋₆alkoxy, haloC₁₋₆alkoxy, C₆₋₁₂aryl, C₁₋₆alkylthio; more preferably halo, hydroxyl, C₁₋₆alkyl, C₁₋₆alkoxy, C₁₋₆alkylthio; optionally wherein two R²⁰ can join to form a ring fused to ring C, wherein said ring formed by two R²⁰ can be selected from the group comprising C₃₋₉cycloalkyl, C₅₋₉cycloalkenyl, C₆₋₁₂aryl, and heterocyclyl, preferably C₅₋₆cycloalkyl, or C₆₋₁₀aryl, and wherein said ring formed by two R²⁰ can be unsubstituted or substituted with one or more R⁴⁰, wherein each R⁴⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably halo, hydroxyl, C₁₋₁₀alkyl; C₁₋₁₀alkoxy, or C₁₋₆alkylthio; preferably C₁₋₆alkyl; more preferably C₁₋₄alkyl; and
n is an integer selected from 0, 1, 2, 3, or 4; preferably 0, 1, 2, or 3, preferably 0, 1, or 2, more preferably 0 or 1, yet more preferably 0.
40. A process for the synthesis of a compound of formula (I) a stereoisomer or racemate thereof, comprising the step of contacting a compound of formula (V), a stereoisomer, or racemate thereof; with at least one reagent selected from the group consisting of an alkyl- or cycloalkyl-chloroformate of formula (VI); an urea; a phosgene or derivative thereof; a cyclic carbonate; and carbon dioxide;

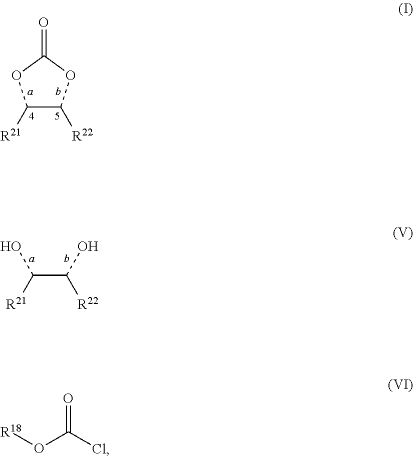

wherein
each dotted bond “a” and “b” independently represents a solid bond, a wedged bond, or a hashed wedged bond;
R²¹ and R²² taken together with the carbon atoms to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; and
R¹⁸ is C₁₋₆alkyl, or C₃₋₆cycloalkyl.
41. A polycarbonate or copolymer thereof comprising a recurring unit of formula (VII1) and/or (VII2),

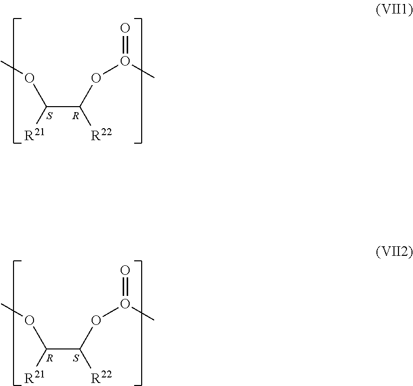

wherein
R²¹ and R²² taken together with the carbon atoms to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.
42. The polycarbonate or copolymer thereof according to statement 41, comprising recurring units each independently represented by the following formula (VIIA) or (VIIB) or (VIIC);

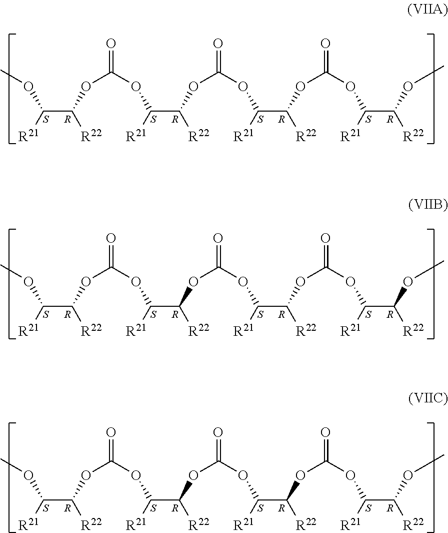

wherein R²¹ and R²² have the same meaning as that defined in statement 41.
43. The polycarbonate or copolymer thereof according to any one of statements 41 or 42, comprising recurring units each independently represented by the following formula (VIIA1)

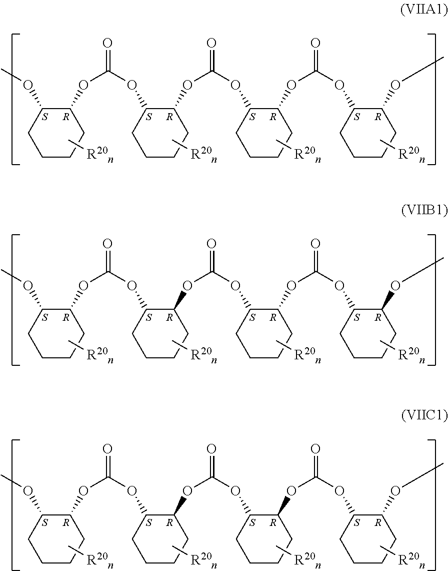

each R²⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino;
n is an integer selected from 0, 1, 2, 3, or 4.
44. A polycarbonate or copolymer thereof comprising recurring units of formula (VII3) or of formula (VII4),

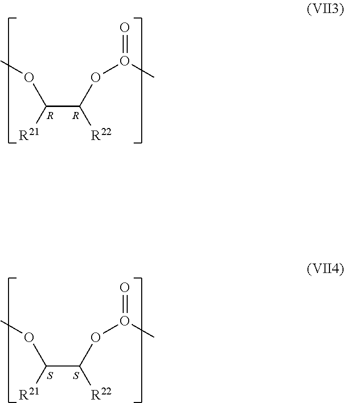

wherein
R²¹ and R²² taken together with the carbon atoms 4 and 5 to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; and
wherein said polycarbonate or copolymer thereof is characterized by a ¹³C{¹H} NMR spectrum comprising only one signal present in the areas ranging from δ 150 to 155 ppm, preferably said polycarbonate or copolymer thereof is characterized by a high resolution (100 MHz or higher)¹³C{¹H} NMR spectrum comprising only one sharp resonance present in the carbonyl areas ranging from δ 150 to 155 ppm.
45. An article comprising a polycarbonate or copolymer thereof according to any one of statements 11-14, 41-44 or a polycarbonate or copolymer thereof prepared according to a process according to any of one of statements 1-9, 16-39.

The present invention provides a process for preparing a polycarbonate, said process comprising homo- or copolymerizing in the presence of at least one catalyst, one or more compounds of formula (I), stereoisomers, racemics, or mixtures thereof; optionally with one or more cyclic esters, stereoisomers, racemics, or mixtures thereof;

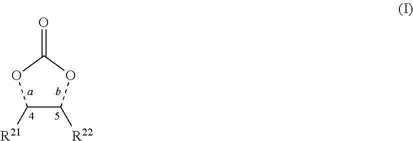

wherein
each dotted bond “a” and “b” independently represents a solid bond ( [symbol] ), a wedged bond ( [symbol] ), or a hashed wedged bond ( [symbol] );
R²¹ and R²² are, each independently, selected from C₁₋₆alkyl, C₃₋₆cycloalkyl, and C₆₋₁₀aryl; preferably C₁₋₆alkyl, or C₃₋₆cycloalkyl; more preferably C₁₋₆alkyl; yet more preferably C₁₋₄alkyl; for example R²¹ and R²² are each independently methyl, ethyl or propyl; for example R²¹ is methyl and R²² is methyl;
or R²¹ and R²² taken together with the carbon atoms 4 and 5 of the cyclic carbonate form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms 4 and 5 of the cyclic carbonate form a ring selected from C₃₋₉cycloalkylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms 4 and 5 of the cyclic carbonate form a ring selected from C₃₋₆cycloalkylene, and C₆₋₁₀arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms 4 and 5 of the cyclic carbonate form C₃₋₆cycloalkylene, preferably a C₄₋₆cycloalkylene, preferably a C₅₋₆cycloalkylene, preferably a cyclohexylene; optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; for example one or more substituents each independently selected from halo, hydroxyl, C₁₋₆alkyl, C₁₋₆alkoxy, or C₁₋₆alkylthio.

In a preferred embodiment, R²¹ and R²² taken together with the carbon atoms 4 and 5 of the cyclic carbonate form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

Preferably, the present invention provides a process for preparing a polycarbonate or copolymer thereof, said process comprising copolymerizing in the presence of at least one catalyst, one or more compounds of formula (I), stereoisomers, racemics, or mixtures thereof; with one or more cyclic esters, stereoisomers, racemics, or mixtures thereof;

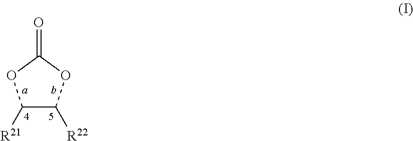

each dotted bond “a” and “b” independently represents a solid bond ( [symbol] ), a wedged bond ( [symbol] ), or a hashed wedged bond ( [symbol] );
R²¹ and R²² taken together with the carbon atoms 4 and 5 of the cyclic carbonate form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino, wherein two of said one or more substituents can join to form a ring fused to said C₃₋₉cycloalkylene, C₅₋₉cycloalkenylene, or C₆₋₁₂arylene; wherein said ring formed by said two substituents is selected from the group comprising C₃₋₉cycloalkyl, C₅₋₉cycloalkenyl, C₆₋₁₂aryl, and heterocyclyl, wherein each group can be unsubstituted or substituted with one or more R⁴⁰, wherein each R⁴⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably each R⁴⁰ is independently selected from halo, hydroxyl, C₁₋₁₀alkyl; C₁₋₁₀alkoxy, or C₁₋₆alkylthio; preferably R⁴⁰ is C₁₋₆alkyl; more preferably C₁₋₄alkyl. Preferably, R²¹ and R²² taken together with the carbon atoms 4 and 5 of the cyclic carbonate form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino. Preferably R²¹ and R²² taken together with the carbon atoms 4 and 5 of the cyclic carbonate form a ring selected from C₅₋₇cycloalkylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino, wherein two of said one or more substituents can join to form a ring fused to said C₅-7cycloalkylene, or C₆₋₁₀arylene ring; preferably R²¹ and R²² taken together with the carbon atoms 4 and 5 of the cyclic carbonate form a ring selected from C₆₋₇cycloalkylene, and C₆₋₁₀arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; wherein two of said one or more substituents can join to form a C₃₋₆cycloalkyl fused to said C₆₋₇cycloalkylene, or C₆₋₁₀arylene ring; preferably R²¹ and R²² taken together with the carbon atoms 4 and 5 of the cyclic carbonate form a cyclohexylene; optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino, for example one or more substituents each independently selected from halo, hydroxyl, C₁₋₆alkyl, C₁₋₆alkoxy, or C₁₋₆alkylthio; wherein two of said substituents can join to form a C₃₋₆cycloalkyl fused to said cyclohexylene ring.

In one embodiment, R²¹ and R²² taken together with the carbon atoms 4 and 5 of the cyclic carbonate form cyclohexylene, phenylene, or cyclopropylene, each optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms 4 and 5 of the cyclic carbonate form a cyclohexylene, or a phenylene, preferably a cyclohexylene.

In an embodiment, the compound of formula (I) can have structural formula (IA) or (IB), preferably structural formula (IA);

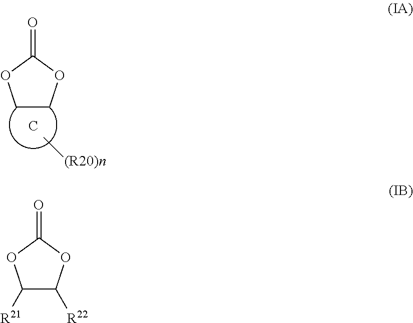

wherein
the ring C is selected from C₃₋₉cycloalkylene, C₅₋₉cycloalkenylene, or C₆₋₁₂arylene; preferably C₄₋₆cycloalkylene, or C₆₋₁₀arylene; more preferably C₅₋₆cycloalkylene;
each R²⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably halo, hydroxyl, C₁₋₁₀alkyl; C₁₋₁₀alkoxy, or C₁₋₆alkylthio; preferably C₁₋₆alkyl; more preferably C₁₋₄alkyl; optionally wherein two R²⁰ can join to form a ring fused to ring C, wherein said ring formed by two R²⁰ can be selected from the group comprising C₃₋₉cycloalkyl, C₅₋₉cycloalkenyl, C₆₋₁₂aryl, and heterocyclyl, preferably C₅₋₉cycloalkyl, or C₆₋₁₀aryl, and wherein said ring formed by two R²⁰ can be unsubstituted or substituted with one or more R⁴⁰, wherein each R⁴⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably halo, hydroxyl, C₁₋₁₀alkyl; C₁₋₁₀alkoxy, or C₁₋₆alkylthio; preferably C₁₋₆alkyl; more preferably C₁₋₄alkyl;
R²¹ and R²² are each independently selected from C₁₋₆alkyl; C₃₋₆cycloalkyl, or C₆₋₁₀aryl, preferably R²¹ and R²² are each independently selected from C₁₋₆alkyl; more preferably C₁₋₄alkyl; and
n is an integer selected from 0, 1, 2, 3, or 4; preferably 0, 1, 2, or 3, preferably 0, 1, or 2, more preferably 0 or 1, yet more preferably 0.

In some embodiments, compound of formula (I) can have one of the structural formula (IA), (IA1), (IA2), (IA3), (IA4), (IB), (IB1), (IB2), (IB3), (IB4), preferably (IA), (IA1), (IA2), (IA3), (IA4),

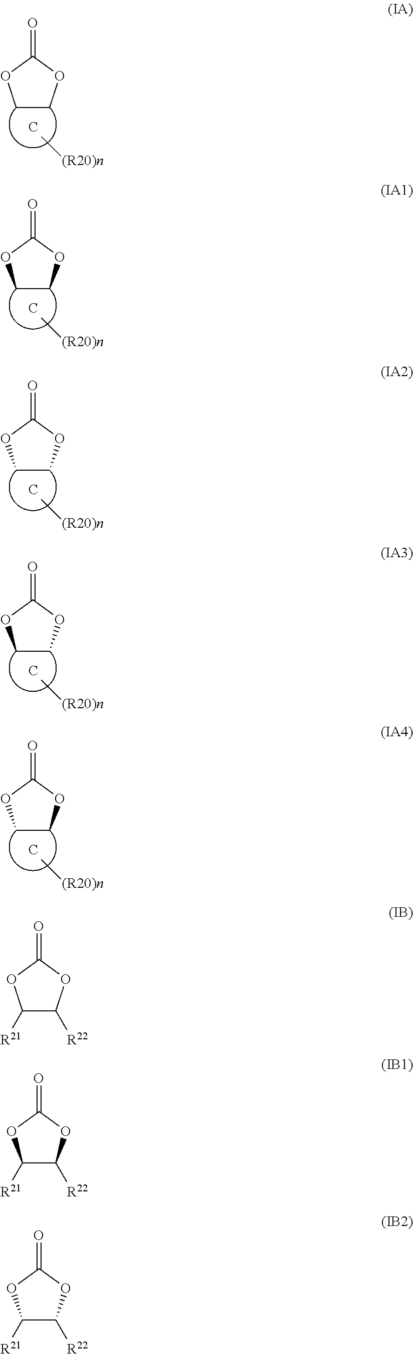

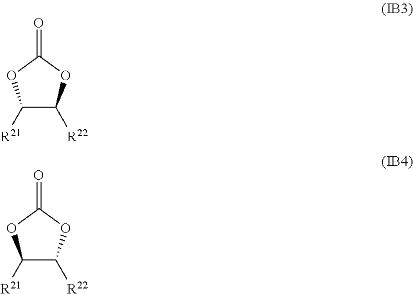

wherein
the ring C is selected from C₃₋₉cycloalkylene, C₅₋₉cycloalkenylene, or C₆₋₁₂arylene; preferably C₄₋₆cycloalkylene, or C₆₋₁₀arylene; more preferably C₅₋₆cycloalkylene;
each R²⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably halo, hydroxyl, C₁₋₁₀alkyl; C₁₋₆alkylthio, or C₁₋₁₀alkoxy, preferably C₁₋₆alkyl, more preferably C₁₋₄alkyl;
R²¹ and R²² are each independently selected from C₁₋₆alkyl; C₃₋₆cycloalkyl, or C₆₋₁₀aryl, preferably R²¹ and R²² are each independently selected from C₁₋₆alkyl; more preferably C₁₋₄alkyl; and
n is an integer selected from 0, 1, 2, 3, or 4; preferably 0, 1, 2, or 3, preferably 0, 1, or 2, more preferably 0 or 1, yet more preferably 0.

Optionally two R²⁰ can join to form a ring fused to ring C, wherein said ring formed by two R²⁰ can be selected from the group comprising C₃₋₉cycloalkyl, C₅₋₉cycloalkenyl, C₆₋₁₂aryl, and heterocyclyl, preferably C₅₋₆cycloalkyl, or C₆₋₁₀aryl, and wherein said ring formed by two R²⁰ can be unsubstituted or substituted with one or more R⁴⁰, wherein each R⁴⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably halo, hydroxyl, C₁₋₁₀alkyl; C₁₋₁₀alkoxy, or C₁₋₆alkylthio; preferably C₁₋₆alkyl; more preferably C₁₋₄alkyl.

In some embodiments, compound of formula (I) can have one of the structural formula (IA), (IA1), (IA2), (IA3), (IA4), wherein the ring C, R²⁰ and n are as defined herein above. In some preferred embodiments, compound of formula (I) can have one of the structural formula (IA), (IA1), (IA2), (IA3), (IA4), wherein the ring C is selected from preferably C₄₋₆cycloalkylene, or C₆₋₁₀arylene; preferably C₅₋₆cycloalkylene; and each R²⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably halo, hydroxyl, C₁₋₁₀alkyl; C₁₋₆alkylthio, or C₁₋₁₀alkoxy, preferably C₁₋₆alkyl, more preferably C₁₋₄alkyl; and n is an integer selected from 0, 1, 2, 3, or 4; preferably 0, 1, 2, or 3, preferably 0, 1, or 2, more preferably 0 or 1, yet more preferably 0.

In some preferred embodiments, compound of formula (I) can have one of the structural formula (IB), (IB1), (IB2), (IB3), (IB4), wherein R²¹ and R²² are each independently selected from C₁₋₆alkyl; or C₆₋₁₀aryl, preferably R²¹ is C₁₋₆alkyl, and R²² is C₁₋₆alkyl; preferably R²¹ is C₁₋₄alkyl, and R²² is C₁₋₄alkyl; preferably R²¹ and R²² are each independently selected from methyl, ethyl, n-propyl.

In some embodiments, compound of formula (I) can be selected from the following compounds of formula (IAa), (IA1a), (IA2a) when n is not 0, (IA3a), (IA4a), (IBa), (IB1a), (IB2a), (IB3a), (IC); preferably (IAa), (IA1a), (IA2a), (IA3a), (IA4a), or (IC);

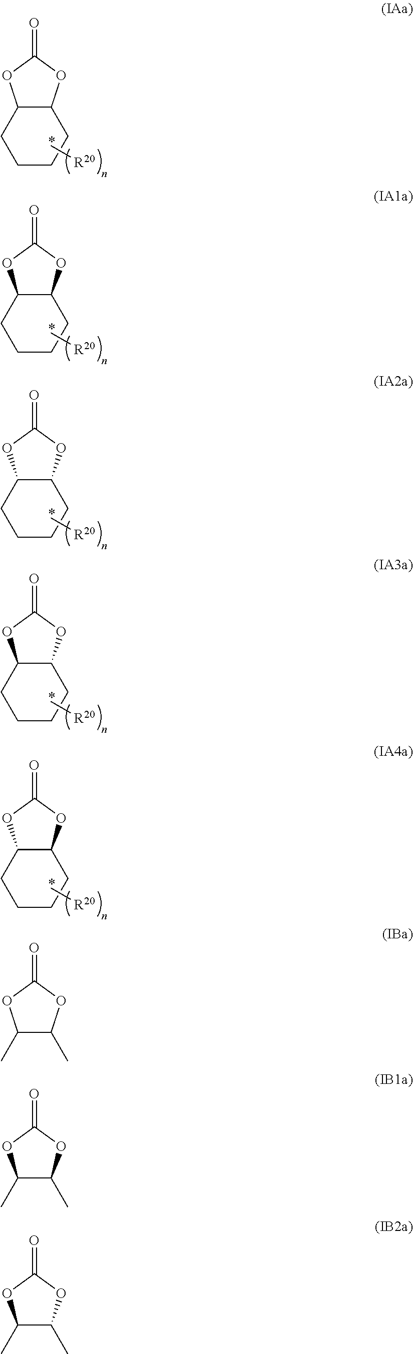

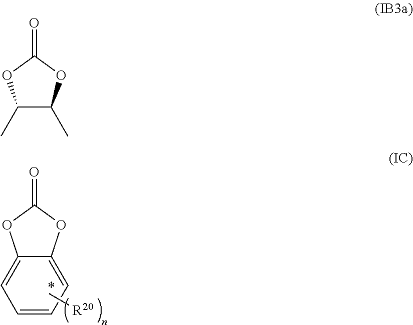

wherein R²⁰ is selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably halo, hydroxyl, C₁₋₆alkyl, haloC₁₋₆alkyl, C₁₋₆alkoxy, haloC₁₋₆alkoxy, C₆₋₁₂aryl, C₁₋₆alkylthio; more preferably halo, hydroxyl, C₁₋₆alkyl, C₁₋₆alkoxy, C₁₋₆alkylthio; and
n is an integer selected from 0, 1, 2, 3, or 4; preferably 0, 1, 2, or 3, preferably 0, 1, or 2, more preferably 0 or 1, yet more preferably 0.

Optionally two R²⁰ can join to form a ring fused to ring C, wherein said ring formed by two R²⁰ can be selected from the group comprising C₃₋₉cycloalkyl, C₅₋₉cycloalkenyl, C₆₋₁₂aryl, and heterocyclyl, preferably C₅₋₆cycloalkyl, or C₆₋₁₀aryl, and wherein said ring formed by two R²⁰ can be unsubstituted or substituted with one or more R⁴⁰, wherein each R⁴⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably halo, hydroxyl, C₁₋₁₀alkyl; C₁₋₁₀alkoxy, or C₁₋₆alkylthio; preferably C₁₋₆alkyl; more preferably C₁₋₄alkyl.

In some embodiments, compound of formula (I) can be selected from the following compounds of formula (IAa), (IA1a), (IA2a), (IA3a), (IA4a), (IC), wherein R²⁰ and n are as defined herein above.

In some embodiments, the polymerization of the present invention can occur with the same compound of formula (I), or with two or more compounds of formula (I) differing in structural formula, in which case a copolymerization takes place. In some embodiments, the polymerization of the present invention can occur with two or more of the same compounds of formula (I) differing only in stereochemistry.

In some embodiments, the copolymerization of the present invention can occur with the same compound of formula (I) and one or more cyclic esters. In some embodiments, the copolymerization of the present invention can occur with two or more compounds of formula (I) differing in structural formula, or differing in stereochemistry, and one or more cyclic ester.

In some other embodiments, the copolymerization of the present invention can occur with the same compound of formula (I) and two or more differing cyclic esters.

As used herein, the terms “cyclic ester” refers to cyclic monoesters, cyclic diesters, cyclic triesters, and the like. Preferred are the cyclic monoesters also known as lactones and cyclic carbonates, and the cyclic diesters also known as glycolide and lactides. In an embodiment, the cyclic ester is selected from the group comprising lactones, cyclic carbonates, glycolides and lactides, each group can be unsubstituted or substituted with one or more substituents each independently selected from C₁₋₆alkyl, and C₁₋₆alkoxy. In an embodiment, the cyclic ester is selected from the group comprising lactones, glycolides and lactides.

Non-limiting examples of suitable cyclic esters can be selected from the group comprising lactide, trimethylene carbonate, glycolide, β-butyrolactone, δ-valerolactone, γ-butyrolactone, γ-valerolactone, 4-methyldihydro-2(3H)-furanone, alpha-methyl-gamma-butyrolactone, ε-caprolactone, 1,3-dioxolan-2-one, propylene carbonate, 4-methyl-1,3-dioxan-2-one, 1,3-doxepan-2-one, 5-C₁₋₄alkoxy-1,3-dioxan-2-one; and mixture thereof; each group can be unsubstituted or substituted with one or more substituents each independently selected from C₁₋₆alkyl and C₁₋₆alkoxy. For example said cyclic esters can be selected from the group comprising lactide, trimethylene carbonate, glycolide, β-butyrolactone, δ-valerolactone, γ-butyrolactone, γ-valerolactone, 4-methyldihydro-2(3H)-furanone, alpha-methyl-gamma-butyrolactone, ε-caprolactone, 4-methyl-1,3-dioxan-2-one, 1,3-doxepan-2-one, and mixture thereof. For example said cyclic esters can be selected from the group comprising lactide, trimethylene carbonate, glycolide, β-butyrolactone, δ-valerolactone, γ-butyrolactone, γ-valerolactone, 4-methyldihydro-2(3H)-furanone, alpha-methyl-gamma-butyrolactone, ε-caprolactone, and mixture thereof. Preferably said cyclic esters can be selected from the group comprising lactide, glycolide, β-butyrolactone, δ-valerolactone, γ-butyrolactone, γ-valerolactone, 4-methyldihydro-2(3H)-furanone, alpha-methyl-gamma-butyrolactone, ε-caprolactone, and mixture thereof. In some embodiment, said cyclic ester is a lactide, such as L-lactide (S,S-lactide), D-lactide (R,R-lactide), or meso-(S,R)-lactide. In some embodiment, said cyclic ester is a cyclic carbonate, such as trimethylene carbonate.

The catalyst employed for this process may have general formula M¹(Y¹, Y², . . . Y^{P})_{q}, in which M¹ is a metal selected from the group comprising the elements of columns 3 to 12 of the periodic table of the elements, as well as the elements Al, Ga, In, Tl, Ge, Sn, Pb, Sb, Ca, Mg and Bi; whereas Y¹, Y², . . . Y^{p} are each substituents selected from the group comprising alkyl with 1 to 20 carbon atoms, aryl having from 6 to 30 carbon atoms, alkoxy having from 1 to 20 carbon atoms, aryloxy having from 6 to 30 carbon atoms, and other oxide, carboxylate, and halide groups as well as elements of group 15 and/or 16 of the periodic table; p and q are integers of from 1 to 6. As examples of suitable catalysts, we may notably mention the catalysts of Al, Sn, Ti, Zr, Zn, and Bi; preferably an alkoxide or a carboxylate and more preferably Sn(Oct)₂, Al(OiPr)₃, Ti(OiPr)₄, Ti(2-ethylhexanoate)₄, Ti(2-ethylhexyloxide)₄, Zr(OiPr)₄, Bi(neodecanoate)₃, (2,4-di-tert-butyl-6-(((2-(dimethylamino)ethyl)(methyl)amino)methyl)phenoxy)(ethoxy)zinc, or Zn(lactate)₂.

In one embodiment, the catalyst is an organometallic catalyst of formula (II) or (Ill),

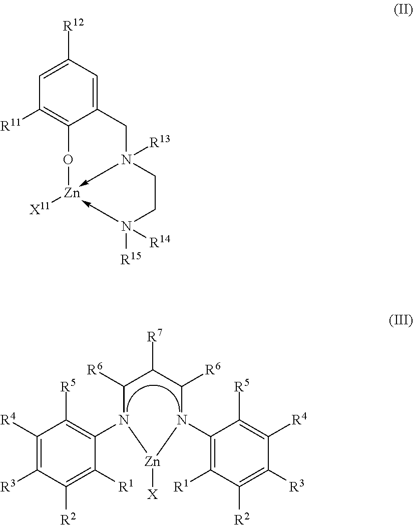

wherein
R¹, R², R³, R⁴, R⁵, R⁶, and R⁷ are each independently selected from the group consisting of hydrogen, optionally substituted C₁₋₁₂alkyl, and an inert functional group (e.g., —CN), and wherein two or more of said groups can be linked together to form one or more rings; preferably R¹, R², R³, R⁴, R⁵, R⁶, and R⁷ are each independently selected from the group consisting of hydrogen, optionally substituted C₁₋₈alkyl, and an inert functional group (e.g., —CN), and wherein two or more of said groups can be linked together to form one or more rings; preferably R¹, R², R³, R⁴, R⁵, R⁶, and R⁷ are each independently selected from the group consisting of hydrogen, or optionally substituted C₁₋₆alkyl; preferably R¹, R², R³, R⁴, R⁵, R⁶, and R⁷ are each independently selected from the group consisting of hydrogen, or optionally substituted C₁₋₄alkyl; preferably R², R³, R⁴ and R⁷ are each independently H, and R¹, R⁵, and R⁶, are each independently C₁₋₆alkyl;
X is —N(SiR²⁷₃)₂, C₁₋₁₂alkyl, C₁₋₁₂alkoxy, —NR⁹R¹⁰ or —BH₄; preferably X is —N(SiR²⁷₃)₂, C₁₋₆alkyl, C₁₋₆alkoxy, or —NR⁹R¹⁰; preferably X is —N(SiR²⁷₃)₂, C₁₋₆alkyl, C₁₋₆alkoxy, or —NR⁹R¹⁰; preferably X is —N(SiR²⁷₃)₂;
each R²⁷ is independently selected from hydrogen and C₁₋₆alkyl;
each R⁹ and R¹⁰ is independently selected from hydrogen and C₁₋₆alkyl;
R¹¹ and R¹² are each independently C₁₋₁₀alkyl; preferably, R¹¹ and R¹² are each independently C₁₋₆alkyl; preferably, R¹¹ and R¹² are each independently C₁₋₄alkyl; for example, R¹¹ and R¹² can be each independently selected from the group consisting of methyl, ethyl, n-propyl, i-propyl, 2-methyl-ethyl, n-butyl, i-butyl and t-butyl; preferably, R¹¹ and R¹² can be each independently selected from the group consisting of methyl, ethyl, i-propyl and t-butyl; for example R¹¹ and R¹² can be each independently selected from i-propyl or t-butyl; preferably, R¹¹ and R¹² are t-butyl;
R¹³, R¹⁴, and R¹⁵ are each independently C₁₋₁₀alkyl; preferably, R¹³, R¹⁴ and R¹⁵ are each independently C₁₋₆alkyl; preferably R¹³, R¹⁴ and R¹⁵ are each independently C₁₋₄alkyl, for example, R¹³, R¹⁴ and R¹⁵ can be each independently selected from the group consisting of methyl, ethyl, n-propyl, i-propyl, 2-methyl-ethyl, n-butyl, i-butyl and t-butyl; for example, R¹³, R¹⁴ and R¹⁵ can be each independently selected from the group consisting of methyl, ethyl, i-propyl and t-butyl; for example, R¹³, R¹⁴ and R¹⁵ are each independently selected from methyl or ethyl; preferably, R¹³, R¹⁴ and R¹⁵ are each independently methyl; or
R¹³ and R¹⁴ are covalently bound to each other and are each a methylene and R¹⁵ is C₁₋₁₀alkyl; preferably R¹⁵ is C₁₋₆alkyl; preferably, R¹⁵ is C₁₋₄alkyl; for example R¹⁵ can be selected from the group consisting of methyl, ethyl, n-propyl, i-propyl, 2-methyl-ethyl, n-butyl, i-butyl and t-butyl; for example R¹⁵ can be selected from the group consisting of methyl, ethyl, i-propyl and t-butyl; for example R¹⁵ can be selected from methyl or ethyl; for example R¹⁵ can be methyl;
X¹¹ is selected from C₁₋₁₀alkyl, —OR¹⁶, and —N(SiR¹⁷₃)₂;
R¹⁶ is C₁₋₁₀alkyl; and
each R¹⁷ is independently selected from hydrogen and C₁₋₆alkyl;
preferably, X¹¹ is selected from C₁₋₆alkyl, —OR¹⁶, or —N(SiR¹⁷₃)₂, R¹⁶ is C₁₋₆alkyl, and each R¹⁷ is independently selected from hydrogen and C₁₋₆alkyl; preferably, X¹¹ is selected from C₁₋₄alkyl, —OR¹⁶, or —N(SiR¹⁷₃)₂, R¹⁶ is C₁₋₄alkyl, and each R¹⁷ is independently hydrogen or C₁₋₄alkyl; for example X¹¹ can be selected from the group consisting of methyl, ethyl, n-propyl, i-propyl, 2-methyl-ethyl, n-butyl, i-butyl and t-butyl, or —OR¹⁶, or —N(SiR¹⁷₃)₂, R¹⁶ can be selected from the group consisting of methyl, ethyl, n-propyl, i-propyl, 2-methyl-ethyl, n-butyl, i-butyl and t-butyl, and each R¹⁷ can be independently selected from the group consisting of hydrogen, methyl, ethyl, n-propyl, i-propyl, 2-methyl-ethyl, n-butyl, i-butyl and t-butyl; preferably, X¹¹ can be selected from the group consisting of methyl, ethyl, i-propyl and n-butyl, or —OR¹⁶, R¹⁶ can be selected from the group consisting of methyl, ethyl, i-propyl and t-butyl; preferably, X¹¹ can be selected from —OR¹⁶, R¹⁶ can be selected from the group consisting of methyl, ethyl, i-propyl and t-butyl; preferably, X¹¹ can be —OR¹⁶, and R¹⁶ is ethyl.

In the present description, an inert functional group is defined as a group containing one or several heteroatoms selected from O, N, S or halogen, that is(are) not reactive in the polymerization system neither as an initiating species nor as a chain transfer agent.

In one embodiment, R¹¹ and R¹² are each independently C₁₋₆alkyl, preferably C₁₋₄alkyl.

In one embodiment, R¹³, R¹⁴ and R¹⁵ are each independently C₁₋₆alkyl, preferably C₁₋₄alkyl.

In one embodiment, X¹¹ is selected from C₁₋₆alkyl, —OR¹⁶, or —N(SiR¹⁷₃)₂, R¹⁶ is C₁₋₆alkyl, and each R¹⁷ is independently selected from hydrogen and C₁₋₄alkyl.

In one embodiment, the catalyst is selected from [(NNO)ZnEt], [BDI]Zn(N(SiMe₃)₂), [BDI]Zn(Et), and {[BDI]Zn(OR³⁰)}₂, wherein R³⁰ is C₁₋₆alkyl; preferably the catalyst is [(NNO)ZnEt].

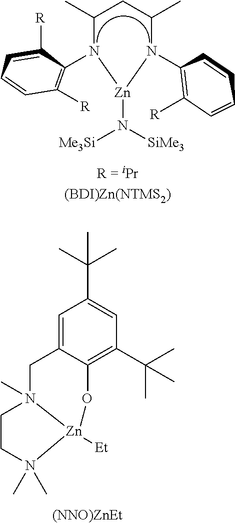

Suitable catalyst also include metal amides such as bis(trimethylsilyl)amides such as Y[N(SiMe₃)₂]₃; [Y]+L1; and [Y]+L2, wherein L1 corresponds to the tetradentate ligand depicted below and L2 corresponds to the tetradentate ligand depicted below

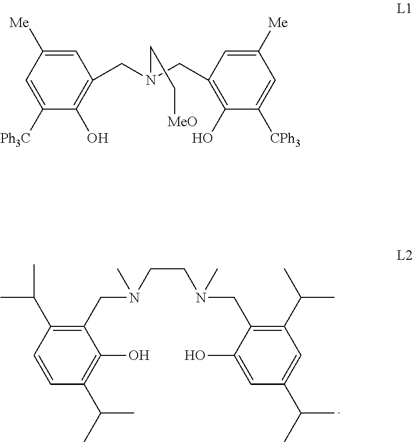

In one embodiment, the catalyst is selected from complexes of formulae M(OSO₂CF₃)ₘ, M(N(OSO₂CF₃)₂)ₘ, M(R²³C(O)CR²³₂C(O)R²³)ₘ, and (R²⁴CO₂)ₘM, wherein M is a metal of Group 2, 3, including the lanthanide series, or Group 12, 13, 15, wherein each R²³ is independently an optionally substituted C₁₋₁₂alkyl, wherein each R²⁴ is independently a perfluorinated C₁₋₁₂alkyl or an aryl, and wherein m is the valence of M.

In one embodiment, all R²³ are the same and are selected from CH₃ and CF₃, or R²⁴ is selected from (C₆F₅), (CF₃), and CF₃(CF₂)ₚ, wherein p is an integer ranging between 1 and 6.

In one embodiment, the catalyst is a Lewis acidic metal salt selected from Al(OTf)₃, Al(NTf₂)₃, Mg(OTf)₂, Ca(OTf)₂, Zn(OTf)₂, Sc(OTf)₃, Bi(OTf)₃, Fe(acac)₃, Al(OCOCF₃)₃, Al(hfacac)₃, Zn(OCOCF₃)₂, Zn(BF₄)₂, Zn(acac)₂, Zn(hfacac)₂, and Zr(acac)₄.

Non-limiting examples of suitable catalyst, include catalyst selected from the group comprising dimeric phosphazene bases, or amines or guanidines, organic acids, sparteine, thiourea-amino derivatives, N-heterocyclic carbenes, 4-(dialkylamino)pyridines, amidines, phosphorus based phosphines, phosphazenium derivatives and phosphazenes, strong and weak Bronsted acids, as described in M Fèvre, J Vignolle, Y Gnanou, and D Taton, Organocatalyzed Ring-Opening Polymerizations, 2012 Elsevier B.V.; and in Chem. Soc. Rev., 2013, 42, 2142.

In one embodiment, the catalyst can be selected from 1,8-diazabicyclo[5.4.0]undec-7-ene (DBU), 4-dimethylaminopyridine (DMAP), 1,5,7-triazobicyclo-[4,4,0]dec-5-ene (TBD), and tert-butylimino-1,3-dimethylperhydro-1,3,2-diazaphosphine (BEMP); Y[N(SiMe₃)₂]₃; [Y]+L1; or [Y]+L2; preferably TBD, BEMP; Y[N(SiMe₃)₂]₃; [Y]+L1; or [Y]+L2; preferably TBD, BEMP.

In one embodiment, the process is performed in the presence of a compound of formula (IV),

R⁸—OH (IV)

wherein R⁸ is selected from the group consisting of C₁₋₂₀alkyl, C₆₋₃₀aryl, and C₆₋₃₀arylC₁₋₂₀alkyl optionally substituted by one or more substituents selected from the group consisting of halogen, hydroxyl, and C₁₋₆alkyl. Preferably, R⁸ is selected from C₃₋₁₂alkyl, C₆₋₁₀aryl, and C₆₋₁₀arylC₃₋₁₂alkyl, optionally substituted by one or more substituents, each independently selected from the group consisting of halogen, hydroxyl, and C₁₋₆alkyl; preferably, R⁸ is selected from C₃₋₁₂alkyl, C₆₋₁₀aryl, and C₆₋₁₀arylC₃₋₁₂alkyl, optionally substituted by one or more substituents, each independently selected from the group consisting of halogen, hydroxyl and C₁₋₄alkyl. The alcohol can be a polyol such as diol, triol or higher functionality polyhydric alcohol. The alcohol may be derived from biomass such as for instance glycerol or 1,3-propanediol or any other sugar-based alcohol such as for example erythritol. The alcohol can be used alone or in combination with another alcohol.

In one embodiment, the compound of formula (IV) is selected from the group comprising 1-octanol, isopropanol, 1,3-propanediol, trimethylolpropane, 2-butanol, 3-buten-2-ol, 1,3-butanediol, 1,4-butanediol, 1,6-hexanediol, 1,7-heptanediol, benzyl alcohol, 4-bromophenol, 1,4-benzenedimethanol, and (4-trifluoromethyl)benzyl alcohol; preferably, said compound of formula (IV) is selected from 1-octanol, isopropanol, and 1,4-butanediol.

In one embodiment, the molar ratio of compound of formula (IV) versus the catalyst is of at least 0. For example, the molar ratio of the co-initiator of the polymerization (compound of formula (IV)) to the catalyst can be of from about 0 to about 1000, for example from about 1 to about 1000, for example of from about 1 to about 100, for example of from about 1 to about 5.

In some embodiments, the present process allows to tailor the molecular weight of the polycarbonate by adapting the molar ratio of compound of formula (IV) versus the catalyst.

In an embodiment, the process is performed in the presence or absence (in bulk) of solvent. In one embodiment, the process is performed in the presence of a solvent. In a preferred embodiment, the solvent is selected from an aliphatic or aromatic hydrocarbon, an ether, a halogenated solvent, and a C₁₋₆alkyl carbonate. In another embodiment, the present process is performed in bulk.

The process can be performed with or without solvent, or can be performed in a minimum amount of solvent. In an embodiment, said minimum amount of solvent can be the solvent necessary to dissolve the catalyst. The solvent can be an aromatic or aliphatic hydrocarbon, an ether, or an halogenated solvent such as chlorinated solvent.

In an embodiment, the solvent is selected from an alkane such as hexane or heptane; an aromatic hydrocarbon such as toluene; an ether such as tetrahydrofuran (THF); and a chlorinated solvent such dichloromethane; a C₁₋₆alkyl carbonate such as dimethyl carbonate; preferably the solvent is an aromatic hydrocarbon, such as toluene.

In one embodiment, the process is performed at a temperature of at least 20° C. In some embodiments, the process is performed at a temperature of at most 150° C. For example, the process can be performed, preferably at a temperature of at least 20° C. and at most 150° C., for example, the process can be performed, preferably at a temperature of at least 40° C. and at most 120° C., for example at least 50° C. and at most 100° C., for example at a temperature of at least 50° C. and at most 90° C., for example at a temperature of between 50° C. and 85° C., for example at a temperature of between 55° C. and 85° C.

In an embodiment, the ratio of the compounds of formula (I) to the catalyst to the compound of formula (IV) can be from 25:1:0 to 5000:1:0, from 25:1:1 to 5000:1:50. In an embodiment, the ratio of the cyclic ester to the compounds of formula (I) to the catalyst to the compound of formula (IV) can be for example 100:100:1:1.

In some embodiments, the process can be carried out with unpurified compound of formula (I) optionally with an unpurified cyclic ester monomer. The process can also be carried out with the resulting (co-)polymer being crystallized one or more times in solvent and dried under vacuum before use. The solvent used during crystallization can be the same or different from the solvent used during the polymerization process.

In an embodiment, the process can be performed by contacting said compound of formula (I) optionally with the cyclic ester monomer with the catalyst and optionally a co-initiator, in a reactor equipped with an agitator, for instance a high viscosity agitator.

In an embodiment, the process can be performed by contacting compound of formula (I), the catalyst, optionally the cyclic ester, and optionally the co-initiator, optionally in the presence of a solvent. In some embodiments, the process can be performed by contacting the catalyst, and the co-initiator, and subsequently adding compound of formula (I), optionally the cyclic ester, optionally in the presence of a solvent.

In an embodiment, the process can be performed by contacting compound of formula (I), optionally the cyclic ester, the catalyst, and optionally the co-initiator, under inert atmosphere, for example in the presence of argon or nitrogen.

The present invention also encompasses a process for the synthesis of a compound of formula (I) as defined in any one of the embodiments herein, a stereoisomer or a racemate thereof, comprising the step of contacting a compound of formula (V), a stereoisomer, or a racemate thereof; with at least one reagent selected from the group comprising an alkyl- or cycloalkyl-chloroformate of formula (VI); an urea; a phosgene or derivative/substitute of phosgene, e.g. triphosgene; a cyclic carbonate; and a carbon dioxide;

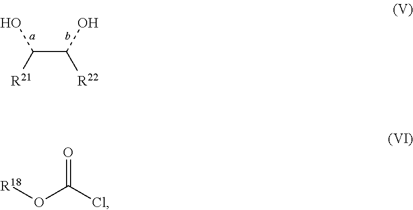

wherein
R¹⁸ is C₁₋₆alkyl or C₃₋₆cycloalkyl; and
each R²¹, R²², and the dotted bonds “a” and “b” are, independently, as defined in any one of the embodiments herein.

In some embodiments, R¹⁸ is C₁₋₆alkyl; and

R²¹ and R²² are each independently selected from C₁₋₆alkyl, C₃₋₆cycloalkyl, and C₆₋₁₀aryl; preferably C₁₋₆alkyl, or C₃₋₆cycloalkyl; more preferably C₁₋₆alkyl; yet more preferably C₁₋₄alkyl;
or R²¹ and R²² taken together with the carbon atoms to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms to which they are attached form a ring selected from C₅₋₆cycloalkylene, and C₆₋₁₀arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms to which they are attached form a ring selected from C₅₋₆cycloalkylene, and phenylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms to which they are attached form a C₅₋₆cycloalkylene, preferably a cyclohexylene; optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; for example one or more substituents each independently selected from halo, hydroxyl, C₁₋₆alkyl, C₁₋₆alkoxy, or C₁₋₆alkylthio.

In some preferred embodiments, R¹⁸ is C₁₋₄alkyl; and R²¹ and R²² are each independently selected from C₁₋₆alkyl, C₃₋₆cycloalkyl, and C₆₋₁₀aryl; preferably C₁₋₆alkyl, or C₃₋₆cycloalkyl; more preferably C₁₋₆alkyl; yet more preferably C₁₋₄alkyl.

Preferably, the present invention provides a process for the synthesis of a compound of formula (I) a stereoisomer or racemate thereof, comprising the step of contacting a compound of formula (V), a stereoisomer, or racemate thereof; with at least one reagent selected from the group consisting of an alkyl- or cycloalkyl-chloroformate of formula (VI); an urea; a phosgene or derivative thereof; a cyclic carbonate; and carbon dioxide;

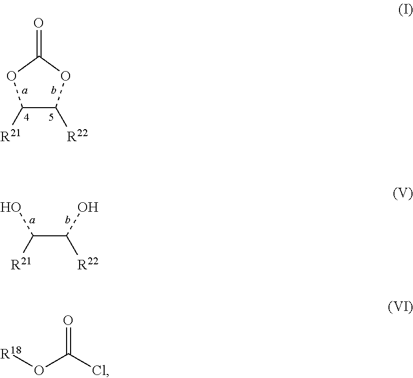

wherein
each dotted bond “a” and “b” independently represents a solid bond, a wedged bond, or a hashed wedged bond;
R²¹ and R²² taken together with the carbon atoms 4 and 5 of the cyclic carbonate form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino, R¹⁸ is C₁₋₆alkyl, or C₃₋₆cycloalkyl, preferably C₁₋₆alkyl.

In some preferred embodiments, R¹⁸ is C₁₋₄alkyl; and R²¹ and R²² taken together with the carbon atoms to which they are attached form a ring selected from C₃₋₉cycloalkylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms to which they are attached form a ring selected from C₅₋₆cycloalkylene, and C₆₋₁₀arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms to which they are attached form a ring selected from C₅₋₆cycloalkylene, and phenylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms to which they are attached form C₄-6cycloalkylene, preferably a cyclohexylene; optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; for example one or more substituents each independently selected from halo, hydroxyl, C₁₋₆alkyl, C₁₋₆alkoxy, or C₁₋₆alkylthio.

In an embodiment, said compound of formula (VI) is a C₁₋₆alkyl chloroformate, such as ethyl chloroformate.

In an embodiment, the reaction can be performed in the presence of a suitable base such as a trialkylamine, e.g. triethylamine.

In an embodiment, the reaction can be performed in the presence of a suitable solvent such as tetrahydrofuran, diethylether or other ethers.

The reaction can be performed at a temperature ranging from 0° C. to 60° C., for example at room temperature.

The present invention also encompasses a polycarbonate or copolymer thereof comprising a recurring unit of formula (VII1) and/or (VII2),

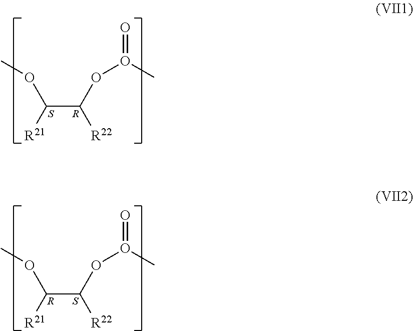

wherein
each R²¹ and R²² are, independently, selected from C₁₋₆alkyl, C₃₋₆cycloalkyl, and C₆₋₁₀aryl; or R²¹ and R²² taken together with the carbon atoms 4 and 5 to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

In some embodiments, R²¹ and R²² are each independently selected from C₁₋₆alkyl, C₃₋₆cycloalkyl, and C₆₋₁₀aryl; preferably C₁₋₆alkyl, or C₃₋₆cycloalkyl; more preferably C₁₋₆alkyl; yet more preferably C₁₋₄alkyl; for example R²¹ and R²² are each independently methyl, ethyl or propyl; preferably R²¹ is methyl and R²² is methyl.

Preferably, the present invention also encompasses a polycarbonate or copolymer thereof comprising a recurring unit of formula (VII1) and/or (VII2), wherein

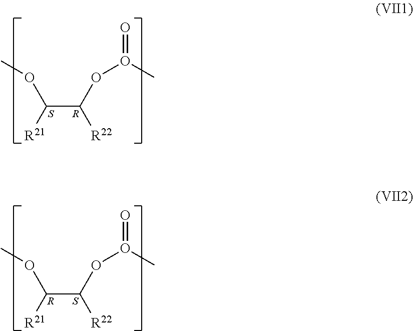

R²¹ and R²² taken together with the carbon atoms to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

In some embodiments, R²¹ and R²² taken together with the carbon atoms to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms to which they are attached form a ring selected from C₃₋₉cycloalkylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms to which they are attached form a ring selected from C₃₋₆cycloalkylene, and C₆₋₁₀arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms to which they are attached form C₃₋₆cycloalkylene, preferably a C₄₋₆cycloalkylene, preferably a C₅₋₆cycloalkylene, preferably a cyclohexylene; optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; for example one or more substituents each independently selected from halo, hydroxyl, C₁₋₆alkyl, C₁₋₆alkoxy, or C₁₋₆alkylthio.

In some embodiment, said polycarbonate or copolymer thereof comprises recurring units each independently represented by the following formula (VIIA) or (VIIB) or (VIIC);

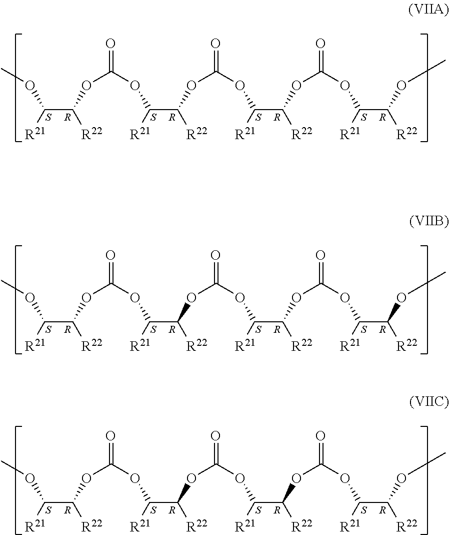

wherein R²¹ and R²² have the same meaning as that defined in any one of the embodiments herein. In some embodiments, R²¹ and R²² are each independently, selected from C₁₋₆alkyl, C₃₋₆cycloalkyl, and C₆₋₁₀aryl; preferably C₁₋₆alkyl, or C₃₋₆cycloalkyl; more preferably C₁₋₆alkyl; yet more preferably C₁₋₄alkyl; for example methyl, ethyl or propyl; for example R²¹ and R²² are each methyl;
or R²¹ and R²² taken together with the carbon atoms 4 and 5 of the cyclic carbonate form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms 4 and 5 of the cyclic carbonate form a ring selected from C₃₋₉cycloalkylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms 4 and 5 of the cyclic carbonate form a ring selected from C₃₋₆cycloalkylene, and C₆₋₁₀arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably R²¹ and R²² taken together with the carbon atoms 4 and 5 of the cyclic carbonate form C₃₋₆cycloalkylene, optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; for example one or more substituents each independently selected from halo, hydroxyl, C₁₋₆alkyl, C₁₋₆alkoxy, or C₁₋₆alkylthio.

In a preferred embodiment, R²¹ and R²² taken together with the carbon atoms 4 and 5 of the cyclic carbonate form a cyclohexylene optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

In a preferred embodiment, R²¹ and R²² are each independently selected from C₁₋₄alkyl, for example methyl, ethyl or propyl; for example R²¹ and R²² are each methyl.

In some embodiment, said polycarbonate or copolymer thereof comprises recurring units each independently represented by the following formula (VIIA1) or (VIIB1) or (VIIC1);

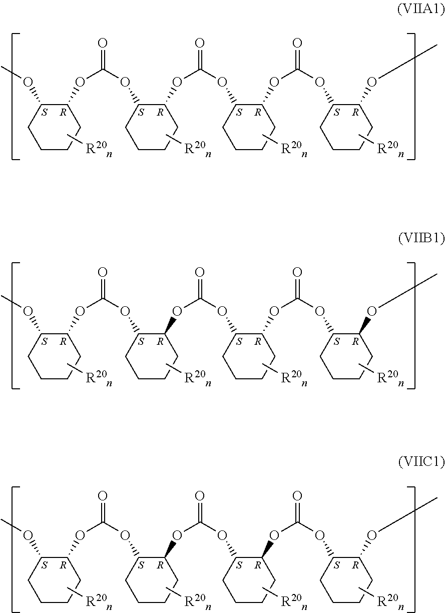

wherein R²⁰ is selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; preferably halo, hydroxyl, C₁₋₆alkyl, haloC₁₋₆alkyl, C₁₋₆alkoxy, haloC₁₋₆alkoxy, C₆₋₁₂aryl, C₁₋₆alkylthio; more preferably halo, hydroxyl, C₁₋₆alkyl, C₁₋₆alkoxy, C₁₋₆alkylthio; and
n is an integer selected from 0, 1, 2, 3, or 4; preferably 0, 1, 2, or 3, preferably 0, 1, or 2, more preferably 0 or 1, yet more preferably 0.

In some embodiment, said polycarbonate or copolymer thereof comprises recurring units each independently represented by the following formula (VIIA2) or (VIIB2) or (VIIC2);

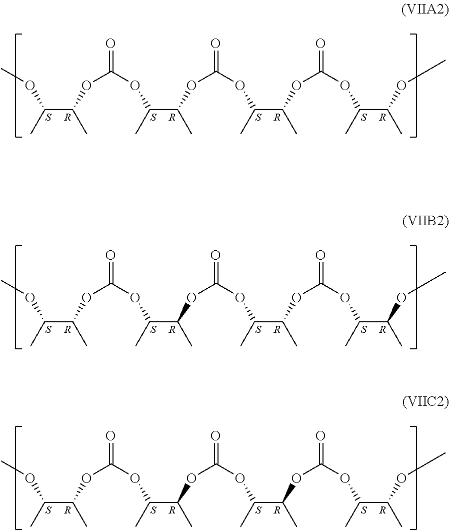

The present invention also encompasses a polycarbonate or copolymer thereof comprising recurring unit of formula (VII3) and/or (VII4),

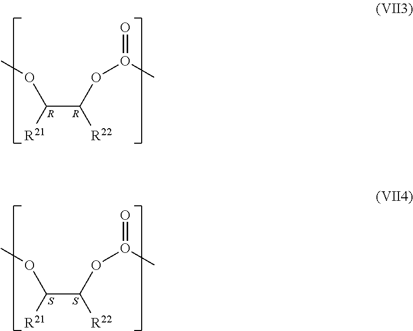

wherein
each R²¹ and R²² are, independently, selected from C₁₋₆alkyl, C₃₋₆cycloalkyl, and C₆₋₁₀aryl; or R²¹ and R²² taken together with the carbon atoms 4 and 5 to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; and
wherein said polycarbonate or copolymer thereof is characterized by a ¹³C NMR spectrum comprising only one signal present in the areas ranging from δ 150 to 155 ppm. In an embodiment, said polycarbonate or copolymer thereof also comprises less than 2% of monomer having at least one chiral center of inverse configuration compared to the enantiopure monomers. This percentage can be measured after hydrolysis of the polymer, by chiral chromatography of the vicinal diol resulting from the hydrolysis.

Preferably, the present invention also encompasses a polycarbonate or copolymer thereof comprising recurring units of formula (VII3) or of formula (VII4),

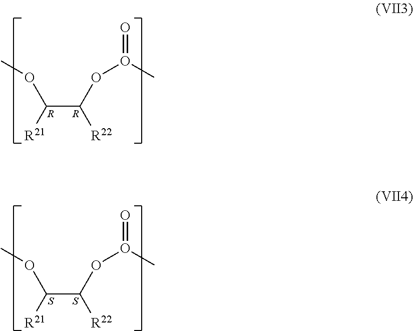

wherein
R²¹ and R²² taken together with the carbon atoms 4 and 5 to which they are attached form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; and
wherein said polycarbonate or copolymer thereof is characterized by a ¹³C{¹H} NMR spectrum comprising only one signal present in the areas ranging from δ 150 to 155 ppm, preferably characterized by a high resolution (100 MHz or higher)¹³C{¹H} NMR spectrum comprising only one sharp resonance present in the carbonyl areas ranging from δ 150 to 155 ppm. In an embodiment, said polycarbonate or copolymer thereof also comprises less than 2% of monomer having at least one chiral center of inverse configuration compared to the enantiopure monomers. This percentage can be measured after hydrolysis of the polymer, by chiral chromatography of the vicinal diol resulting from the hydrolysis.

Hydrolysis of the polycarbonate can be performed as described by Nozaki et al. (Nozaki, K.; Nakano, K.; Hiyama, T. J. Am. Chem. Soc. 1999, 121, 11008-11009). An example of protocol is described herein wherein, a round-bottomed flask was charged with PCHC (50 mg), tetrahydrofuran (5 mL), methanol (1 mL) and NaOH in H₂O (2 M, 10 mL). The resulting mixture was stirred at room temperature for 24 h. Then, it was neutralized with aqueous HCl (1 M), and concentrated to 4 ml by evaporation. The solution was then extracted with ethyl acetate (4×5 mL). The combined organic layers were dried over MgSO₄ and concentrated by evaporation. Further purification of the crude product by silica-gel column chromatography (ethyl acetate as an eluent) gave (1R,2R)-cyclohexane-1,2-diol.

In Scheme 1 below, there is provided a classification of poly(cyclohexylene carbonate) according to the relative stereochemistry of the carbon at which the main chain enters the cyclohexylene unit (a), the carbon at which the main chain enters and leaves the cyclohexylene unit (b) and adjacent carbons (c).

The term “diad” refers to two adjacent structural units in a polymer molecule. If the diad consists of two identically oriented units, the diad is called a meso diad [m] reflecting similar features as a meso compound. If the diad consists of units oriented in opposition, the diad is called a racemo diad [r] as in a racemic compound.

The term “isotactic triad [mm]” refers to two adjacent meso diads. The term “syndiotactic triad [rr]” refers to two adjacent racemo diads. The term “heterotactic triad [rm]” is composed of a meso diad adjacent to a racemo diad.

The tem “tetrad” refers to three adjacent diads.

The [m] and [r] assignments used herein represent the relative stereochemistry of the carbons of the cycloalkylene units (like cyclohexylene units of the Scheme 1 below) at which the main chain enters (a), not the relative stereochemistry of the two carbons on either side of the carbonate unit (c). Therefore a racemic diad ([r]) represents two entire monomer units that have been incorporated in the opposite stereochemical orientation.

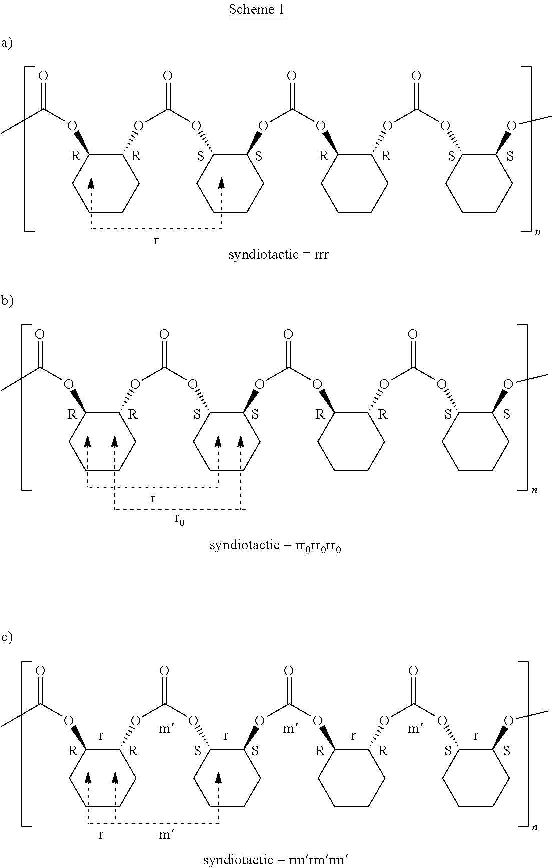

Scheme 2 describes the different limit microstructures reachable from enantiopure (R,R), rac (R,R/S,S) and meso (R,S) cyclohexylene carbonate and the corresponding nomenclature considering the absolute configuration at the C4 carbon atom in the cyclohexylene ring.

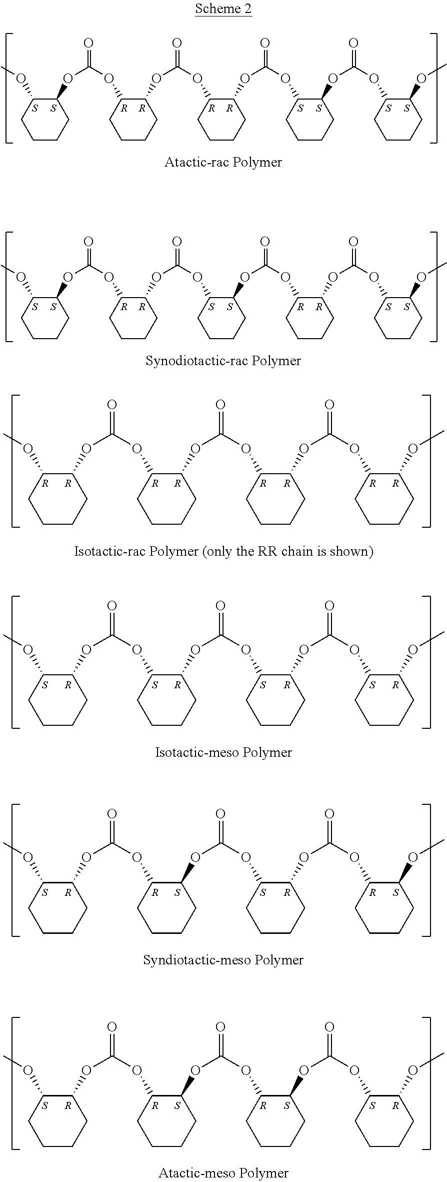

The present invention thereof encompasses polycarbonate as defined in the third aspect of the invention herein comprising isotactic-meso microstructure, or syndiotactic-meso microstructure, or atactic-meso microstructure.

The process of the present invention allows control of tacticity: using a trans enantiopure (R,R)—CHC allows to obtain a perfect isotactic polymer; on the other hand, using a cis (R,S)—CHC allows to obtain new architectures (as shown above in Scheme 2).

The present invention also encompasses polycarbonates and copolymers thereof obtainable by any one of the processes of the invention. The present invention also encompasses polycarbonates and copolymers thereof comprising at least one recurring unit of formula (VII1), (VII2), (VII3), or (VII4); obtainable by any one of the processes of the invention. The present invention also encompasses polycarbonates and copolymers thereof comprising at least one recurring unit of formula (VII1), (VII2), (VII3), or (VII4) according to the third or fourth aspect of the invention; obtainable by any one of the processes of the invention. In an embodiment, the invention encompasses random (co-) polymers obtainable by the process according to any one of the embodiments presented herein.

In some embodiments, the inventors found that the (co-)polymers prepared with the process of the invention can have high molar mass and narrow molar mass distribution.

The “mass” or “molar mass distribution” or “dispersity” is defined by the ratio M_{w}/Mₙ of the weight average molecular weight M_{w} to the number average molecular weight Mₙ as determined by Size Exclusion Chromatography.

In an embodiment, the present process allows the preparation of (co-)polymers, with a number average molecular weight Mₙ which can range between about 250 and about 100,000 g/mol. For example the Mₙ of the (co-)polymers obtained can range between about 2,500 and about 35,000 g/mol. Specific Mₙ values of the (co-)polymers obtained are provided in the tables below.

The Mₙ can be measured by any adequate technique known to the skilled persons for molar mass determination, for instance by chromatography such as size exclusion chromatography (SEC, also referred to as gel permeation chromatography, GPC)) in tetrahydrofuran (THF) at 20° C. calibrated with polystyrene standards.

In an embodiment, the mass molar distribution of the (co-)polymers obtained can range between about 1.10 and about 2.00, preferably below 2.00. Specific M_{w}/Mₙ ratios of the (co-)polymers obtained are provided in the tables below.

The process of the present invention allows improved incorporation of compound of formula (I) monomer into the polycarbonate backbone of the resulting polymer or copolymers thereof. The incorporation is calculated as the molar percentage (mol-%) of compound(s) of formula (I) monomer converted to (co-)polymer as follows: [molar amount of compound(s) of formula (I) in the precipitated (co-)polymer obtained after the process divided by the sum of the molar amounts of compound(s) of formula (I) and optionally cyclic ester(s) in the precipitated (co-)polymer obtained after the process]×100. The molar amounts of compound(s) of formula (I) and optionally cyclic ester(s) in the precipitated (co-) polymer obtained after the process can be measured by NMR spectroscopy. Improved conversions of compound(s) of formula (I) to (co-)polymers are reported in the tables below at particular operating conditions, employing specific catalytic systems, and specific cyclic esters.

The present invention also encompasses an article comprising a polycarbonate or copolymer thereof according to the invention, or a polycarbonate or copolymer thereof prepared according to any of the process of the invention.

The present invention can be further illustrated by the following examples, although it will be understood that these examples are included merely for purposes of illustration and are not intended to limit the scope of the invention unless otherwise specifically indicated.

### EXAMPLES

### Example 1

### Preparation of Poly(Cyclohexene Carbonate) (PCHC) by Ring-Opening Polymerization of Trans Racemic Cyclohexene Carbonate ((±)Trans-CHC)

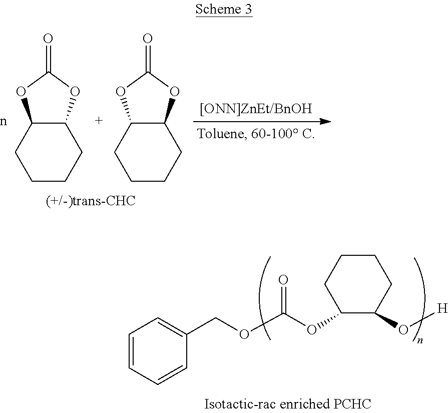

Different ring opening polymerizations (ROP) of (±)-trans-CHC were performed with various catalysts, and reacting conditions. The conditions and results are listed in Table 1. One combination of specific catalyst and reacting conditions is depicted in Scheme 3 above. The polymerization was performed using the following catalytic systems:

DBU, (BDI)Zn(NTMS₂), and [(NNO)ZnEt]. DBU was commercially available from Aldrich. DBU was dried over calcium hydride over 24 h before being distillated under vacuum and stored under inert atmosphere.

(BDI)Zn(NTMS₂) was synthesized as described in Chamberlain B. M., Cheng M., Moore D. R., Ovitt T. M., Lobkovsky E. B., Coates G. W.; J. Am; Chem. Soc., 2001, 123, 3229-3230.

[(NNO)ZnEt] was prepared as described in (Williams C. K., Breyfogle L. E., Choi S. K., Nam W., Young V. G., Hillmeyer M. A., Tolman W. B.; J. Am. Chem. Soc., 2003, 125, 11350-11359).

In some cases, these metallic species were combined to an exogenous protic source, typically an alcohol (benzyl alcohol, BnOH). Benzyl alcohol (from Acros) was distilled over Mg turnings under argon atmosphere and kept over activated 3-4 Å molecular sieves. CDCl₃ was dried over 3-4 Å molecular sieves.

The polymerization was performed as follows: In a glovebox, at room temperature, the catalyst was added to a flask prior to the addition of toluene (volume calculated to have a final concentration in monomer of 2 mol·L⁻¹) and the optional addition of BnOH. The monomer (±)-trans-CHC (prepared as described in Example 3) was then added to the mixture. The mixture was stirred at the listed temperature (Table 1) over an appropriated time period.

Table 1, entries 2-9 were performed in the presence of benzyl alcohol (BnOH) with [(NNO)ZnEt] and PCHCs with a number-average molecular weight (Mn) lower than 22,000 g/mol were synthesized. The molecular weights determined by NMR (Mn_{NMR}) were in good agreement with the theoretical values (Mnₜₕₑₒ). Mn_{NMR} were calculated from the relative intensity of the methylene protons from the benzylic alcohol group (Bn-CH₂—O—PCHC) compared to the one of the methine protons from the CHC units in the polymer (R—O—(CO)—O—CHCH—(C4H8)-O—(CO)—O—R).

The values of molar masses measured by SEC, calibrated from polystyrene standards and unadjusted from their difference in their hydrodynamic volume, remained consistent with Mn_{NMR} and Mnₜₕₑₒ for PCHCs with masses below 22,000 g/mol. Further experiments were performed at 80° C. (Table 1, entries 7-8) and at 60° C. (Table 1, entry 9). Varying the amount of BnOH equivalent (for example, Table 1, entries 12-13) allows a good tailoring of the molar masses. In addition, since the reaction proceeds by immortal ring-opening, the amounts of catalyst used can be minimized. The molecular weights determined by NMR (Mn_{NMR}) and SEC were in perfect agreement with the theoretical values (Mnₜₕₑₒ).

The reactions were performed also with different zinc catalysts, for example: [(BDI)Zn{N(TMS)₂}]. Molecular weights, dispersities, conversions and stereoselectivities obtained from each of the two catalyst systems were found to be similar.

TABLE 1

| Entry | Catalyst | [±CHC]₀:[Cat]₀:[BnOH]₀ | Temp. (° C.) | Time (H) | Conv. CHC (%) | Mnₜₕₑₒ (g/mol) | Mn_{NMR} (g/mol) | Mn_{sec} (g/mol) | $\frac{{M}_{W}}{{M}_{n}}$ |
|---|---|---|---|---|---|---|---|---|---|
| 1 | [(NNO)ZnEt] | 47:1:0 | 100 | 18 | 38 | 2 500 | 22 300 | 19 000 | 1.81 |
| 2 | [(NNO)ZnEt] | 50:1:1 | 100 | 18 | 72 | 5 120 | 6 750 | 7 320 | 1.28 |
| 3 | [(NNO)ZnEt] | 100:1:1 | 100 | 24 | 63 | 8 950 | 9 360 | 7 850 | 1.28 |
| 4 | [(NNO)ZnEt] | 50:1:1 | 100 | 24 | 86 | 6 110 | 7 600 | 7 800 | 1.43 |
| 5 | [(NNO)ZnEt] | 200:1:1 | 100 | 72 | 76 | 21 600 | 17 000 | 8 650 | 1.35 |
| 6 | [(NNO)ZnEt] | 250:1:5 | 100 | 72 | 82 | 5 830 | 7 500 | 5 400 | 1.39 |
| 7 | [(NNO)ZnEt] | 250:1:1 | 80 | 32 | 89 | 31 600 | 20 600 | 21700 | 1.27 |
| 8 | [(NNO)ZnEt] | 100:1:1 | 80 | 3.75 | 89 | 12 650 | 9 800 | 11 000 | 1.64 |
| 9 | [(NNO)ZnEt] | 100:1:1 | 60 | 3 | 91 | 13 100 | 10 300 | 11 400 | 1.33 |
| 10 | [(BDI)Zn{N(TMS)₂}] | 50:1:1 | 100 | 76 | 82 | 5 800 | 7 500 | 6 100 | 1.27 |
| 11 | DBU | 36:1:1 | 60 | 16 | 5 | 250 | — | Not precipitable | |
| 12 | [(NNO)ZnEt] | 250:1:5 | 60 | 4 | 92 | 6 540 | 6540 | 6 200 | 1.15 |
| 13 | [(NNO)ZnEt] | 500:1:5 | 60 | 4 | 87 | 12 400 | 12 100 | 10 850 | 1.07 |

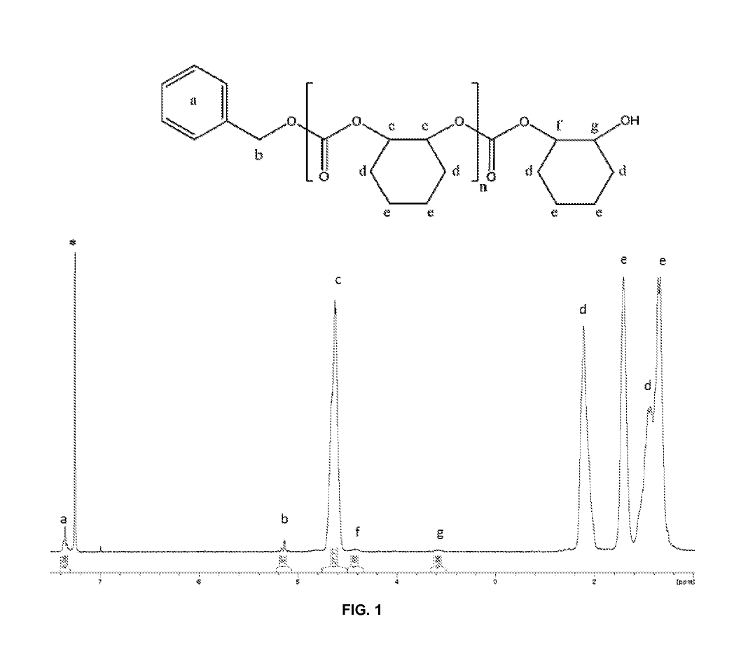

¹H NMR analysis (FIG. 1) of the PCHCs obtained by ROP showed the signals corresponding to the main chain methylene hydrogens (δ 4.63 ppm, OCH(C₄H₈)CHO; δ 2.11 ppm, 1.70 ppm, CH₂CH₂CH; δ 1.46-1.34 ppm, CH₂CH₂CH₂). Benzyloxy resonances (δ 7.35 ppm, OCH₂C₆H₅; 5.14 ppm, OCH₂C₆H₅, respectively) and PCHC—OH (δ 4.40 ppm OCH(C₄H₈)CHOH, δ 3.58 ppm OCH(C₄H₈)CHOH, respectively) in terminal positions on the PCHC were also clearly identified. These data confirm that the polymerization was initiated by benzyl alcohol. Remarkably, there was an absence of signals corresponding to the ether bonds (δ 3.4 ppm), proving the perfect chemoselectivity (within the NMR accuracy) of this process.

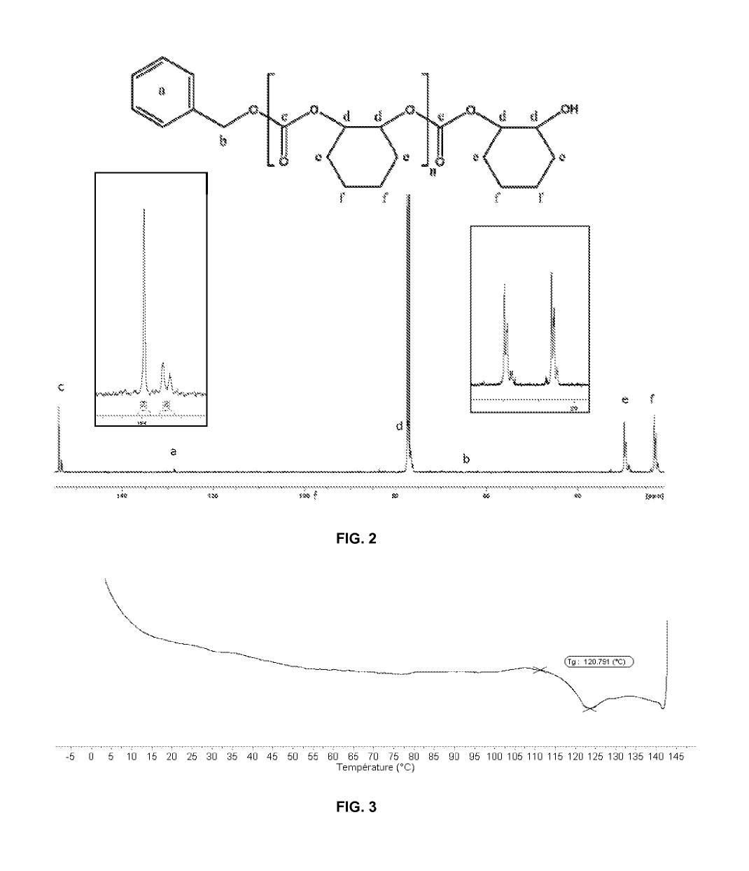

The ¹³C{¹H} NMR spectrum confirmed the chemical structure of PCHC (see FIG. 2). The ¹³C{¹H} NMR spectrum showed all the same signals in the region of carbonates (δ 153.4 and 153.3 ppm) and in the region of alkanes (δ 29.0, 28.7, 22.5 and 22.3 ppm). These signals were attributed to tetrads. The signal at δ 153.9 ppm was assigned to the m-centered tetrads [mmm] [mmr] and [rmr] and the two signals at δ 153.4 ppm and δ 153.3 ppm were assigned to tetrads [mrm]/[rrr]. The integral ratio of the former and latter resonances was found to be 61:39. This polymer appears thus to be enriched in the isotactic-rac microstructure (Scheme 2).

The present process also allows to prepare polycarbonates with an isotacticity close to 100%.

Differential scanning calorimetry (DSC) analyses were performed on a Setaram DSC 131 apparatus calibrated with indium at a rate of 10° C.·min⁻¹, under continuous flow of helium (25 mL·min⁻¹), using aluminum capsules (typically 10 mg of polymer). The thermograms were recorded according to the following cycles: 30° C. to +230° C. at 10° C.·min⁻¹; +230° C. to 30° C. at 10° C.·min⁻¹. The obtained PCHC had a glass transition temperature (Tg) of 121° C. (FIG. 3).

### Example 2

### Preparation of Pure Isotactic (R,R)—PCHC by Ring-Opening Polymerization of Trans (R,R)—CHC

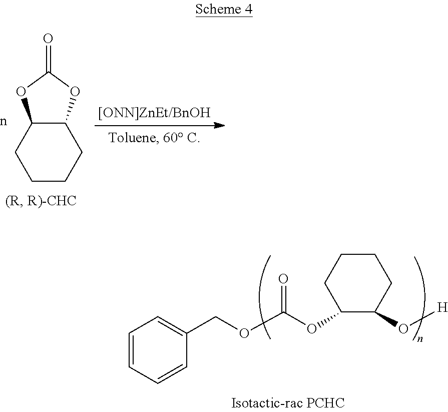

Different ROP of (R,R)—CHC (prepared as described in Example 3) were performed with the zinc complex ([(NNO)ZnEt]) (prepared as described in Example 1). Conditions are depicted in Scheme 4 above. Table 2, entries 1-3 were performed in the presence of benzyl alcohol (BnOH) with [(NNO)ZnEt] and PCHCs with a number-average molecular weight (Mn) lower than 21,000 g·mol⁻¹ were synthesized. The molecular weights determined by NMR (Mn_{NMR}) were in good agreement with the theoretical values (Mnₜₕₑₒ). The values of molar masses measured by SEC in chloroform (the (R,R)PCHCs synthesized were insoluble in THF), calibrated from polystyrene standards and unadjusted for their difference in their hydrodynamic volume, remained coherent with Mn_{NMR} and Mnₜₕₑₒ for PCHCs with masses below 21,000 g·mol⁻¹.

TABLE 2

| Entry | [(R,R)CHC]₀:[(NNO)ZnEt]₀:[BnOH]₀ | Temp. (° C.) | Time (H) | Conv. CHC (%) | Mnₜₕₑₒ (g/mol) | Mn_{NMR} (g/mol) | Mn_{sec} (g/mol) | $\frac{{M}_{W}}{{M}_{n}}$ |
|---|---|---|---|---|---|---|---|---|
| 1 | 100:1:1 | 60 | 1.5 | 94 | 13 360 | 15 800 | 21 000 | 1.34 |
| 2 | 200:1:1 | 60 | 0.8 | 68 | 19 330 | 17 000 | 21 000 | 1.30 |
| 3 | 120:1:1 | 60 | 0.75 | 87 | 14 500 | 10 100 | 12 000 | 1.14 |

¹H NMR analysis of a (R,R)—PCHC (Table 2, Entry 1) showed the same signals as an iso-enriched PCHC (FIG. 1).

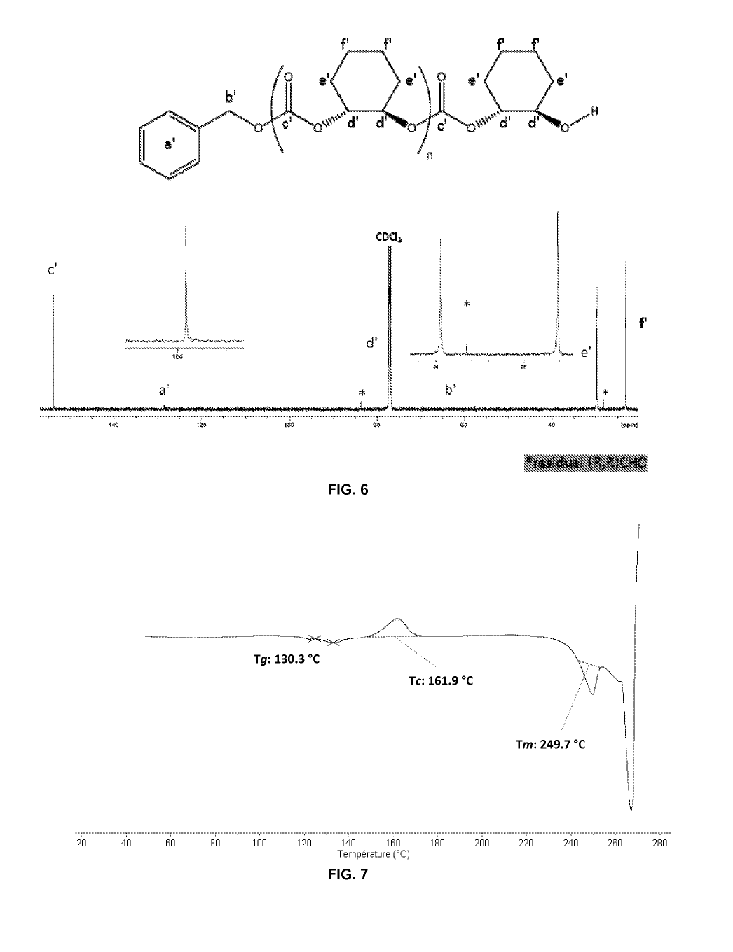

The ¹³C{¹H} NMR spectrum (Table 2, Entry 1) confirmed the chemical structure of PCHC (see FIG. 6). The ¹³C{¹H} NMR spectrum showed only one signal in the region of carbonates (δ 153.95 ppm) which was attributed to m-centered tetrads [mmm] [mmr] and [rmr]. The lack of signals at δ 153.4 ppm and 153.3 ppm indicated that there is no racemo diad in the polymer thus the signal at δ 153.95 ppm in FIG. 6 is only due to [mmm] tetrads. The region of alkanes shows only 2 signals (δ 29.84 and 23.21 ppm) as well.

Differential scanning calorimetry (DSC) analyses were performed on a Setaram DSC 131 apparatus calibrated with indium at a rate of 10° C.·min⁻¹, under continuous flow of helium (25 mL·min⁻¹), using aluminum capsules (typically 10 mg of polymer). The thermograms were recorded according to the following cycles: 30° C. to +280° C. at 10° C.·min⁻¹; +280° C. to 30° C. at 10° C.·min⁻¹. The obtained isotactic (R,R)—PCHC (Table 2, entry 2) had a glass transition temperature (T_{g}) of 130° C., a crystallization temperature (T_{c}) of 162° C. and a melting temperature (Tₘ) of 250° C. (FIG. 7). Once the melting point is reached, the polymer seems to degrade which gives an endothermic peak, and then no signal during the second heating phase.

### Example 3

### Synthesis of a Meso or Racemo-Cyclohexene Carbonate (CHC)

(Meso or racemo) 1.2-cyclohexanediol and ethyl chloroformate were bought from Aldrich and used as received without any purification.

The synthesis of meso or trans-racemic-CHC comprised the transesterification reaction between a cyclohexanediol (meso or trans racemic, respectively) (0.5 M, 1 eq) and ethyl chloroformate (2.25 eq), in the presence of triethylamine (2.25 eq), as set out in Scheme 6. The reaction was carried out in 6 h in tetrahydrofuran at room temperature, obtaining isolated CHC with a yield of 90%. Polymer formation was not observed during the reaction.

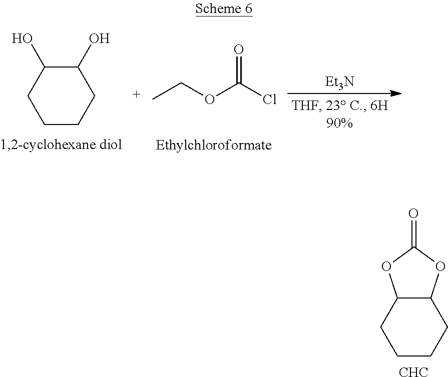

### Example 4

### Preparation of Polycarbonate Copolymer by ROP Copolymerization of (±)-Trans-CHC with L-Lactide

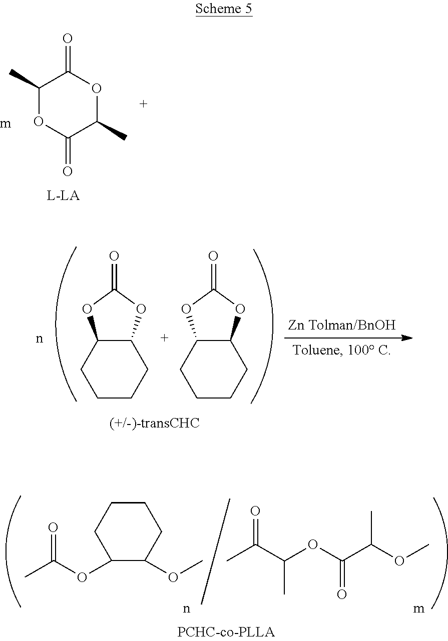

(±)-Trans-CHC was prepared as described in Example 3. L-Lactide (L-LA; technical grade, Total Petrochemicals) was purified by recrystallization from a hot (80° C.) solution of concentrated 2-propanol, followed by two subsequent recrystallizations in hot toluene (105° C.). After purification, L-LA was stored at room temperature under inert atmosphere. The polymerization was performed as illustrated in Scheme 5. The conditions and results of the copolymerization by ROP of (±)-trans-CHC with L-lactide are shown in Table 3.

TABLE 3

| Catalyst | [L-LA]₀:[±CHC]₀:[Cat]₀:[BnOH]₀ | T (° C.) | Time (h) | Conv. CHC (%) | Conv. L-LA (%) | Mnₜₕₑₒ (g/mol) | Mn_{NMR} (g/mol) | Mn_{sec} (g/mol) | $\frac{{M}_{w}}{{M}_{n}}$ |
|---|---|---|---|---|---|---|---|---|---|
| [(NNO)ZnEt] | 100:100:1:1 | 100 | 65 | 78 | 100 | 25 500 | 18 650 | 15 800 | 1.39 |
| (Zn Tolman) | | | | | | | | | |

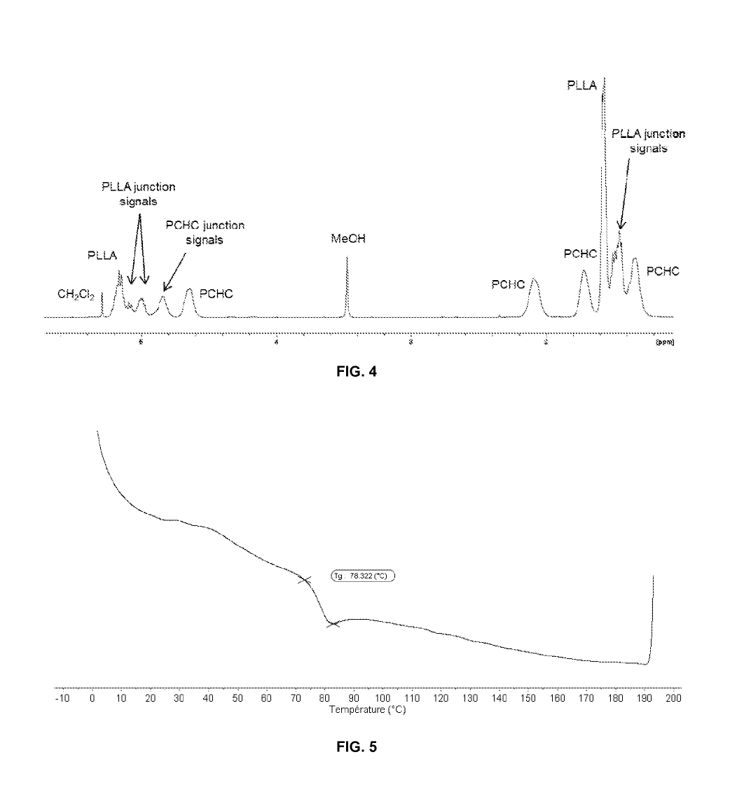

The ¹H NMR spectrum (FIG. 4) showed the formation of a copolymer PLLA-co-PCHC, not a mixture of the two homopolymers PLLA and PCHC. Indeed, the signals were intense and could be clearly identified. The formation of a copolymer and not a mixture of homopolymers, was also supported by the narrow dispersity determined by SEC.

Thermal analysis of this copolymer by DSC (FIG. 5) also suggested the formation of a random copolymer. The measured T_{g} for the copolymer PLLA-co-PCHC was 78° C., between the T_{g} of PLLA (60° C.) and that of the PCHC (116° C.). Differential scanning calorimetry (DSC) analyses were performed on a Setaram DSC 131 apparatus calibrated with indium at a rate of 10° C.·min⁻¹, under continuous flow of helium (25 mL·min⁻¹), using aluminum capsules (typically 10 mg of polymer). The thermograms were recorded according to the following cycles: 30° C. to +230° C. at 10° C.·min⁻¹; +230° C. to 30° C. at 10° C.·min⁻¹.

### Example 5

### Synthesis of 2,3-dimethylcarbonate

2,3-Butanediol and ethyl chloroformate were bought from Aldrich and used as received without any purification. The synthesis of 2,3-dimethylcarbonate comprised the transesterification reaction between the butane-2,3-diol (0.5 M, 1 eq) and ethyl chloroformate (2.25 eq), in the presence of triethylamine (2.25 eq), as set out in scheme 7. The reaction was carried out in 6 h in THF at room temperature. Polymer formation was not observed during the reaction.

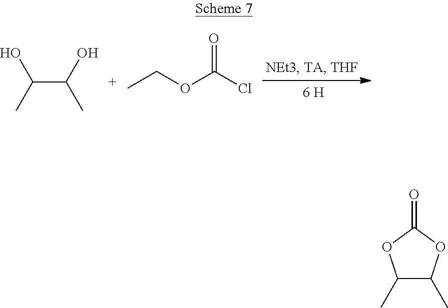

### Example 6

### Preparation of Poly(Cyclohexene Carbonate) (PCHC) by Ring-Opening Polymerization of Rac-Cyclohexene Carbonate (Rac-CHC)

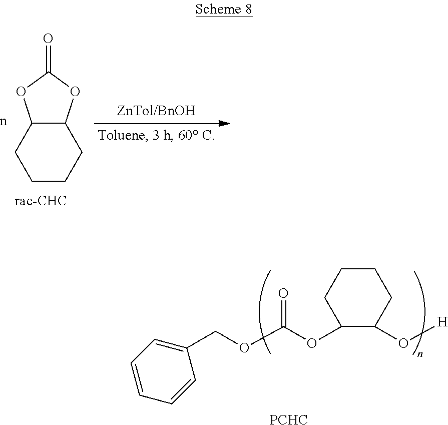

Different ring-opening polymerizations (ROP) of racemic cyclohexene carbonate (rac-CHC) were performed with various catalysts, alcohols and reacting conditions. The conditions and results are listed in Table 4. One combination of specific catalyst and reacting conditions is depicted in Scheme 8. The polymerization was performed using the following catalytic systems: [(NNO)ZnEt], Y[N(SiMe₃)₂]₃, [Y]+L1, [Y]+L2, and TBD.

Y[N(SiMe₃)₂]₃: was commercially available from Strem Chemicals.

[Y]+L1, wherein L1 corresponds to the tetradentate ligand depicted in Scheme 9, was prepared as described in Bouyahyi M, Ajellal N, Kirillov E, Thomas C M, Carpentier J-F. Exploring Electronic versus Steric Effects in Stereoselective Ring-Opening Polymerization of Lactide and β-Butyrolactone with Amino-alkoxy-bis(phenolate)-Yttrium Complexes. Chem Eur J. 2011; 17:1872-1883.

[Y]+L2, wherein L2 corresponds to the tetradentate ligand depicted in Scheme 9, was prepared as described in Kramer J W, Treitler D S, Dunn E W, Castro P M, Roisnel T, Thomas C M, Coates G W. Polymerization of Enantiopure Monomers Using Syndiospecific Catalysts: A New Approach to Sequence Control in Polymer Synthesis. J Am Chem Soc. 2009; 131:16042-16044.

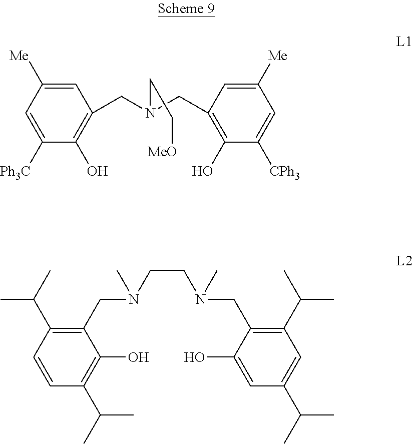

TBD: (1,5,7-triazobicyclo-[4,4,0]dec-5-ene) was commercially available from Aldrich. Isopropyl alcohol was commercially available from Aldrich.

Benzyl alcohol (BnOH) (from Acros) was further treated as described in Example 1.

Typical polymerization procedure: All polymerizations were performed similarly according to the following typical procedure (Table 4, entry 1): In a dry-box, benzyl alcohol (0.97 μL, 9.34 μmol), [(NNO)ZnEt](4.0 mg, 9.34 μmol) and dry toluene (0.10 mL) were placed in a Schlenk flask equipped with a magnetic stir-bar. The mixture was stirred 10 min at room temperature to form the benzyloxide catalyst. rac-CHC (0.133 g, 0.934 mmol, prepared as described in Example 3, and dry toluene (0.13 mL) were then added. The flask and its content were placed under argon and immersed in an oil-bath at 60° C. After stirring over 3 h, the reaction mixture was cooled down to room temperature and three drops of a 1.6 M acetic acid solution in toluene were added to quench the reaction. The solvent was evaporated and an aliquot was taken out to determine the % of CHC conversion by ¹H NMR. The crude polymer was purified by precipitation in cold methanol. This process was repeated twice to ensure complete removal of the catalyst and unreacted CHC. A white powder was obtained following drying under vacuum (0.110 g, 82% isolated yield).

The isolated yield of polymer and the % of conversion of CHC are in agreement. This result clearly indicates that all the CHC converted was transformed to high molecular weight PCHC.

No decarboxylation (as evidenced by the absence of ether signals in ¹H and ¹³C NMR spectra) or formation of non-precipitable oligomers was observed.

TABLE 4

| Entry | Catalyst | ROH | [±CHC]ₒ:[Cat]ₒ:[ROH]ₒ | Temp (° C.) | Time (h) | CHC Conv.^{a} (%) | Mnₜₕₑₒ^{b} (g/mol) | Mn_{NMR}^{c} (g/mol) | Mn_{SEC}^{d} (g/mol) | [symbol] $\frac{{M}_{W}}{( {M}_{n} )}$ |
|---|---|---|---|---|---|---|---|---|---|---|
| 1 | [(NNO)ZnEt] | BnOH | 100:1:1 | 60 | 3 | 88 | 13 100 | 10 300 | 11 700 | 1.1 |
| 1′ | [(NNO)ZnEt] | BnOH | 100:1:1 | 60 | 3 | 91 | 13 100 | 10 300 | 11 400 | 1.2 |
| 2 | Y[N(SiMe₃)₂]₃ | iPrOH | 50:1:1 | 40 | 21 | 28 | 570 | | | |
| 3 | [Y] + L1 | iPrOH | 100:1:1 | 40 | 16 | 13 | 10 100 | | | — |
| 4 | [Y] + L1 | iPrOH | 100:1:1 | 40 | 16 | 56 | 10 100 | | | — |
| 5 | [Y] + L1 | iPrOH | 100:1:1 | 60 | 16 | 70 | 10 100 | 10 600 | 13 100 | 1.2 |
| 6 | [Y] + L2 | iPrOH | 100:1:1 | 60 | 16 | 67 | 9600 | 11 000 | 11 400 | 1.2 |
| 7 | [Y] + L2 | iPrOH | 50:1:1 | 40 | 16 | 83 | 6000 | 6700 | 8600 | 1.1 |
| 8 | TBD | BnOH | 100:1:1 | 60 | 1 | 75 | 10 700 | 8300 | 7800 | 1.1 |
| 9 | TBD | BnOH | 100:1:1 | 60 | 19 | 93 | 13 200 | 16 100 | 10 000 | 1.5 |
| 10 | TBD | BnOH | 250:1:5 | 60 | 4 | 85 | 6000 | 6100 | 6100 | 1.2 |

^{a}Calculated from the ¹H NMR of the crude product.
^{b}Theoretical molar mass value of PCHC calculated from the relation: M_{n,theo} = M_{rac-CHC} × [rac-CHC]₀/[ROH]₀ × conversion_{rac-CHC} + M_{ROH} with M_{rac-CHC} = 142 g · mol⁻¹, M_{iPrOH} = 60 g · mol⁻¹, M_{BnOH} = 108 g · mol⁻¹.
^{c}NMR molar mass value of PCHC calculated from the integral value ratio of the signals of alkoxide methylene or methine end-group hydrogens to internal methine hydrogens.
^{d}Experimental number average molar mass and dispersity values determined by SEC in THF at 30° C. using polystyrene standards.

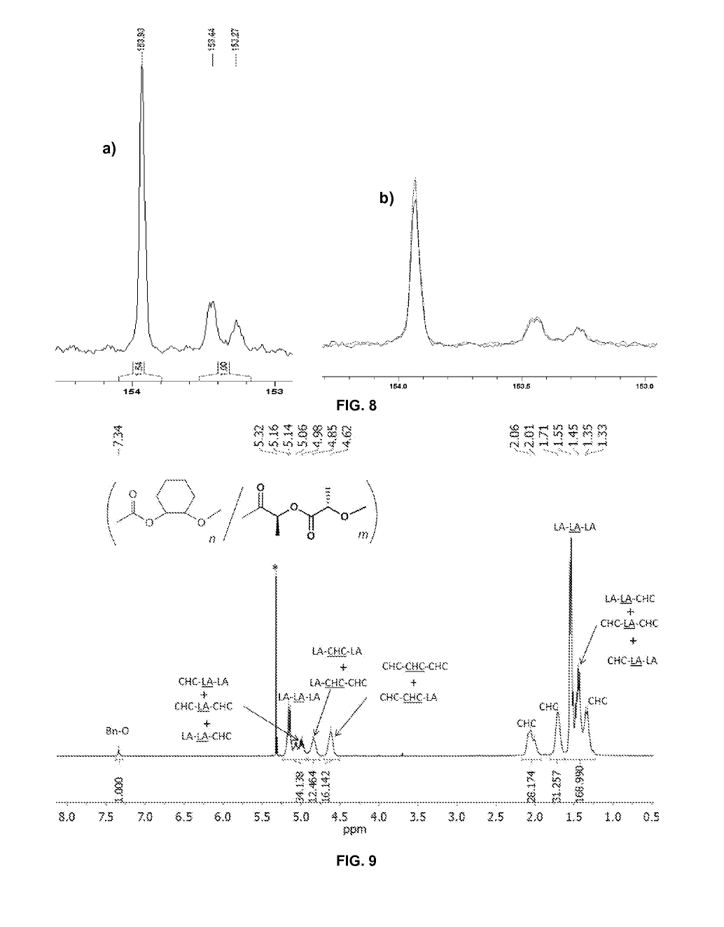

FIG. 8 shows the carbonyl regions of the ¹³C{¹H} NMR spectra (100 MHz, CDCl₃, 25° C.) of PCHC samples synthesized by ROP of rac-CHC using two different catalysts: a) [(NNO)ZnEt] (Table 1, entry 1′) and b) Yttrium complexes (Table 1, entry 5 (lighter line) and entry 6 (darker line)).

NMR analyses revealed that all the catalytic systems investigated consistently gave the same extent of isotacticity (Pₘ=ca. 60-76%) as determined by integration of the mm/mr/rr triads in the methylene region of the ¹³C{¹H} NMR spectra. This might reflect a chain-end control mechanism.

### Example 7

### Preparation of PCHC-PLLA Copolymers by ROP Copolymerization of Rac-CHC with L-Lactide

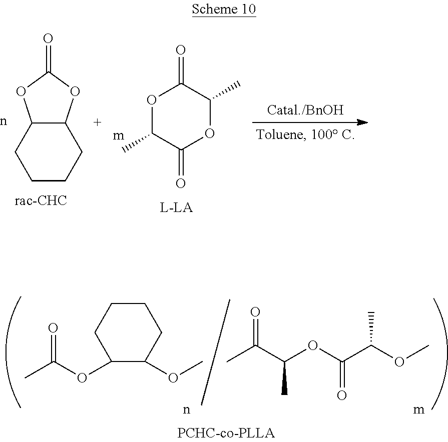

Different copolymerizations of racemic cyclohexene carbonate (rac-CHC) and L-Lactide (L-LA) (Scheme 10) were performed with various catalysts, monomer concentrations and reacting conditions. The conditions and results are listed in Table 5. The copolymerizations were performed using the following catalytic systems: [(NNO)ZnEt] or TBD.

Typical procedure for CHC/L-LA simultaneous copolymerization was as follows: All copolymerizations were performed similarly to the following typical procedure (Table 5, entry 4). In a dry-box, benzyl alcohol (0.97 μL, 9.34 μmol), [(NNO)ZnEt](4.0 mg, 9.34 μmol) and dry toluene (0.10 mL) were placed in a Schlenk flask equipped with a magnetic stir-bar. The mixture was stirred 10 min at room temperature to form the corresponding zinc alkoxide catalyst. rac-CHC (0.066 g, 0.467 mmol, prepared as described in Example 3), L-lactide (0.202 g, 1.401 mmol, prepared as described in Example 4) and dry toluene (0.37 mL) were then added under argon. The flask and its content were immersed in an oil-bath at 100° C. After stirring over 6 h, the reaction mixture was cooled down to room temperature. Three drops of a 1.6 M acetic acid solution in toluene were added to quench the reaction. The solvent was evaporated and after determination of the monomers' conversions by ¹H NMR analysis of the crude product, the resulting mixture was dissolved in CH₂Cl₂ (10 mL) and precipitated in cold methanol (50 mL). This process was repeated twice to ensure complete removal of the catalyst and the unreacted monomers. A white copolymer was finally obtained following drying under vacuum.

TABLE 5

| | | [rac-CHC]₀: | | CHC | L-LA | | | | |
|---|---|---|---|---|---|---|---|---|---|
| | | [LLA]₀: | Time | Conv.^{a} | Conv.^{a} | Mnₜₕₑₒ^{b} | Mn_{NMR}^{c} | Mn_{SEC}^{d} | |
| Entry | Catalyst | [Catal.]₀:[BnOH]₀ | (H) | (%) | (%) | (g/mol) | (g/mol) | (g/mol) | [symbol] _{M} |
| 1 | [(NNO)ZnEt] | 50:150:1:1 | 6 | 80 | 95 | 26200 | nd | 29300 | 1.6 |
| 2 | [(NNO)ZnEt] | 100:100:1:1 | 65 | 78 | 100 | 25 500 | nd | 15 800 | 1.4 |
| 3 | [(NNO)ZnEt] | 150:50:1:1 | 6 | 23 | 80 | 10700 | nd | 11 300 | 1.3 |
| 4 | [(NNO)ZnEt] | 50:150:1:1 | 6 | 91 | 100 | 28200 | 26100 | 30 100 | 1.5 |
| 5 | [(NNO)ZnEt] | 100:100:1:1 | 24 | 93 | 100 | 27700 | 24300 | 20 300 | 1.5 |
| 6 | [(NNO)ZnEt] | 150:50:1:1 | 6 | 86 | 100 | 25600 | 23100 | 19 200 | 1.4 |
| 7 | [(NNO)ZnEt] | 50:150:1:1 | 6 | 86 | 100 | 27800 | 22700 | 25 900 | 1.5 |
| 8 | [(NNO)ZnEt] | 100:100:1:1 | 24 | 82 | 100 | 26200 | 18300 | 13 300 | 1.5 |
| 9 | [(NNO)ZnEt] | 150:50:1:1 | 6 | 77 | 100 | 23700 | 19900 | 14 100 | 1.4 |
| 10 | TBD | 50:150:1:1 | 6 | 77 | 100 | 27200 | 26400 | 13 800 | 1.4 |
| 11 | TBD | 100:100:1:1 | 24 | 42 | 100 | 20500 | 23500 | 13 800 | 1.3 |
| 12 | TBD | 150:50:1:1 | 6 | 26 | 100 | 12800 | 11800 | 6400 | 1.3 |

^{a}Calculated from the ¹H NMR of the crude product.
^{b}Theoretical molar mass value of PCHC-co-PLLA calculated from the relation: M_{n,theo} = M_{rac-CHC} × [rac-CHC]₀/[BnOH]₀ × conversion_{rac-CHC} + M_{LLA} × [LLA]₀/[BnOH]₀ × conversion _{LLA} + M_{BnOH} with M_{rac-CHC} = 142 g · mol⁻¹, M_{LLA} = 144 g · mol⁻¹, M_{BnOH} = 108 g · mol⁻¹.
^{c}NMR molar mass value of the copolymer calculated from the integral value ratio of the signals of alkoxide methylene or methine end-group hydrogens to internal methine hydrogens.
^{d}Experimental number average molar mass and dispersity values determined by SEC in THF at 30° C. using polystyrene standards.
The concentrations of CHC and LLA in all experiments are 4M except for entries 7-9 which are 2M.

Similarly as in Example 3, using [(NNO)ZnEt]catalyst, yields very high CHC incorporation (up to 93%) into PCHC/PLLA copolymer.

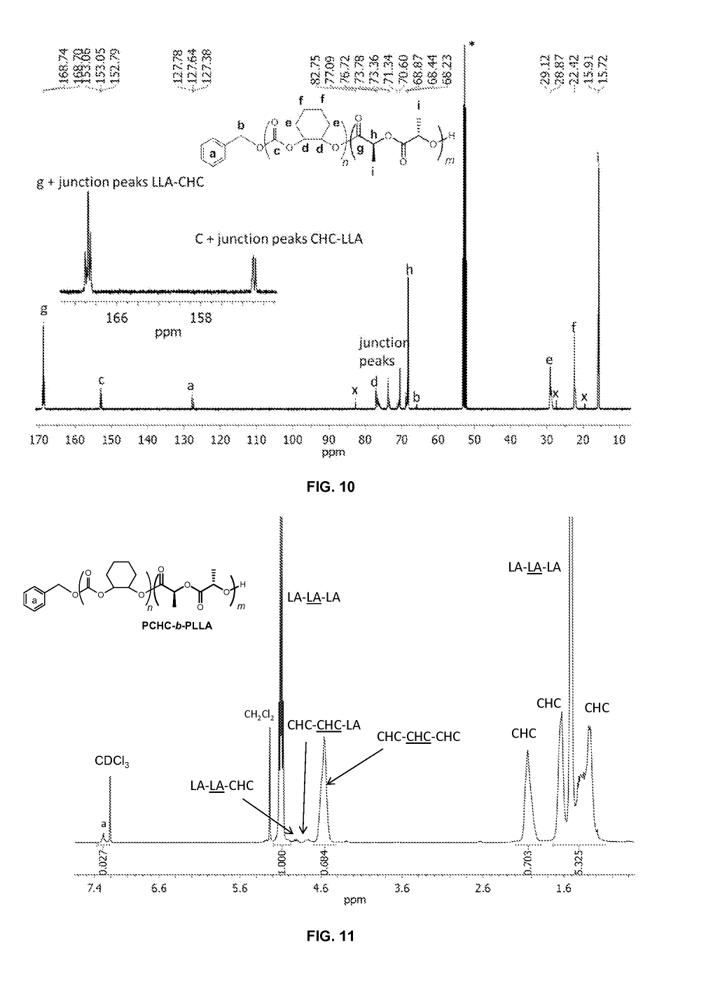

FIGS. 9 and 10 show the ¹H NMR (FIG. 9) and ¹³C{¹H} NMR (FIG. 10) spectra of the copolymer from entry 8 of Table 5 (wherein x stands for unreacted CHC, and * stands for residual solvent resonances).

### Example 8

### Preparation of PCHC-PLLA Diblock Copolymers by Sequential Copolymerization of Rac-CHC and L-Lactic Acid

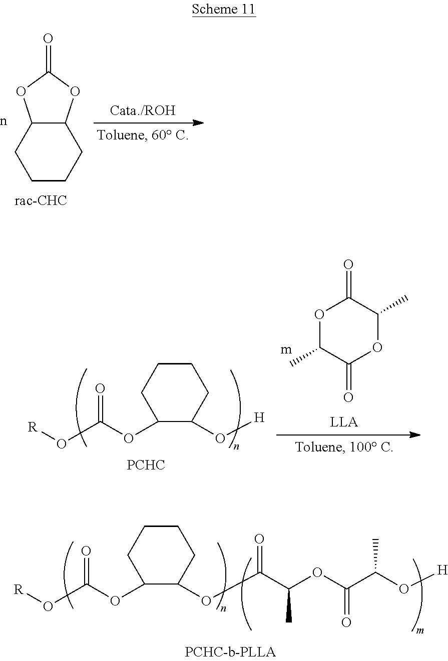

The process was performed by sequential copolymerization (addition of CHC prior to LLA), as depicted in Scheme 11. Different copolymerizations were performed with various catalysts, alcohols, monomer concentrations and reaction time during the copolymerization of the rac-CHC. The conditions and results are listed in Table 6. The copolymerizations were performed using the following catalytic systems: [(NNO)ZnEt], TBD, or Y[N(SiMe₃)₂]₃.

Typical procedure for CHC/L-LA sequential copolymerization (addition of CHC prior to LLA). All copolymerizations were performed similarly to the following typical procedure (Table 6, Entry 2).

The only differences lie in the nature of the catalyst, of the alcohol, in the initial concentrations in monomers, and in the reaction time during the homopolymerization of the racemic cyclohexene carbonate (rac-CHC). In a dry-box, benzyl alcohol (0.97 μL, 9.34 μmol), [(NNO)ZnEt](4.0 mg, 9.34 μmol) and dry toluene (0.10 mL) were placed in a Schlenk flask equipped with a magnetic stir-bar. The mixture was stirred 10 min at room temperature to form the corresponding alkoxide catalyst. rac-CHC (0.133 g, 0.934 mmol, prepared as described in Example 3) and dry toluene (0.23 mL) were then added under argon. The flask and its contents were immersed in an oil-bath at 60° C. After stirring over 6 h, L-lactide (LLA, 0.135 g, 0.934 mmol, prepared as described in Example 4) and dry toluene (0.24 mL) were added under argon; the reaction mixture was stirred over 2 h at 100° C., and was cooled down to room temperature. Three drops of a 1.6 M acetic acid solution in toluene were added to quench the reaction. The solvent was evaporated and the crude polymer was dissolved in CH₂Cl₂ (10 mL) and precipitated in cold methanol (50 mL). This process was repeated twice to ensure complete removal of the catalyst and the unreacted monomers. A white polymer was finally obtained following drying under vacuum.

TABLE 6

| | | | [rac-CHC]₀: | | | | | | | | |
|---|---|---|---|---|---|---|---|---|---|---|---|
| | | | [L-LA]₀: | CHC | LLA | PCHC | | PCHC-b-PLLA | | | |
| | | | [Catalyst₀: | Conv.^{a} | Conv.^{a} | Mnₜₕₑₒ^{b} | Mn_{NMR} | Mnₜₕₑₒ^{c} | Mn_{NMR}^{d} | Mn_{SEC}^{e} | |
| Entry | Catalyst | ROH | [ROH]₀ | (%) | (%) | (g/mol) | (g/mol) | (g/mol) | (g/mol) | (g/mol) | [symbol] _{M} |
| 1 | [(NNO)ZnEt] | BnOH | 50:150:1:1 | 85 | 98 | 6200 | 4200 | 27300 | 31600 | 28000 | 1.7 |
| 2 | [(NNO)ZnEt] | BnOH | 100:100:1:1 | 86 | 97 | 12300 | 11900 | 26300 | 28000 | 25300 | 1.5 |
| 2′ | [(NNO)ZnEt] | BnOH | 100:100:1:1 | 86 | 95 | 12300 | nd | 26000 | 25000 | nd | nd |
| 3 | [(NNO)ZnEt] | BnOH | 150:50:1:1 | 77 | 91 | 16500 | 19300 | 23100 | 23400 | 1 100 | 1.2 |
| 4 | TBD^{e} | BnOH | 50:150:1:1 | 91 | 96 | 6600 | 6900 | 27300 | nd | 2 600 | 1.5 |
| 5 | TBD^{e} | BnOH | 100:100:1:1 | 91 | 94 | 13000 | 16500 | 26600 | 29000 | 17000 | 1.4 |
| 6 | TBD^{e} | BnOH | 150:50:1:1 | 91 | 100 | 19500 | 17300 | 26700 | 31700 | 17100 | 1.4 |
| 7 | Y[N(SiMe₃)₂]₃^{f} | iPrOH | 50:150:1:1 | 87 | 97 | 6200 | nd | 27200 | nd | 28600 | 1.7 |
| 8 | Y[N(SiMe₃)₂]₃^{f} | iPrOH | 100:100:1:1 | 77 | 97 | 11000 | nd | 25000 | 24900 | 22900 | 1.6 |
| 9 | Y[N(SiMe₃)₂]₃^{f} | iPrOH | 150:50:1:1 | 71 | 100 | 15200 | nd | 22400 | 23600 | 20300 | 1.6 |

^{a}Calculated from the ¹H NMR of the crude product. ^{b}Theoretical molar mass value of PCHC calculated from the relation: M_{n,theo} = M_{rac-CHC} × [rac-CHC]₀/ROH]₀ × conversion_{rac-CHC} + M_{ROH} with M_{rac-CHC} = 142 g · mol⁻¹, M_{BnOH} = 108 g · mol⁻¹, M_{iPrOH} = 60 g · mol⁻¹.
^{c}Theoretical Mₙ value of PCHC-b-PLLA calculated from the relation: M_{n,theo} {M_{rac-CHC} × [rac-CHC]₀/[ROH]₀ × conversion_{rac-CHC}} + {M_{LLA} × [LLA]₀/[ROH]₀ × conversion_{LLA}} + M_{ROH}.
^{d}NMR molar mass value of PCHC/PLLA copolymer calculated from the integral value ratio of the signals of alkoxide methylene or methine end-group hydrogens to internal methine hydrogens.
^{e}Experimental number average molar mass and dispersity values determined by SEC in THF at 30° C. using polystyrene standards.
^{e,f}The homopolymerization reaction time of rac-CHC is 24 h and 16 h, respectively.

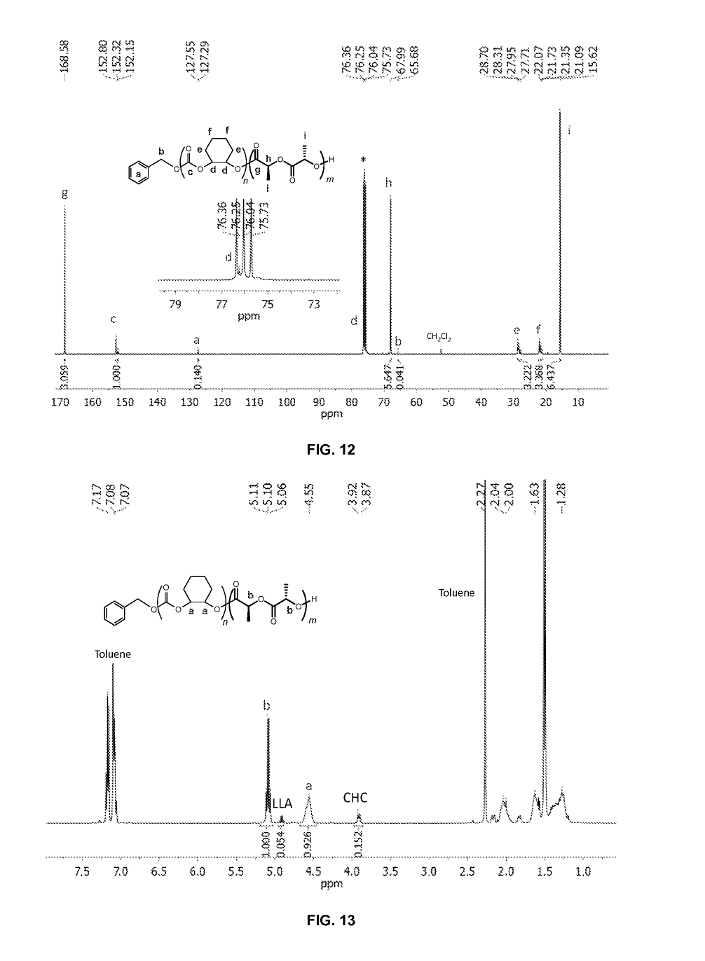

The ¹H NMR spectrum (FIG. 11) of the PCHC-b-PLLA copolymer prepared according to Table 6, entry 2 showed the signals of the PCHC (CH at δ 4.61 ppm) and the PLLA (CH at δ 5.15 ppm) main chain CH in a 0.68:1.00: =0.68 ratio, which is quite different from the CHC and LLA conversions ratio (0.86:0.97=0.89). This is likely due to the aliquot of PCHC taken out for analysis to determine the CHC conversion prior to the addition LLA. The ¹³C{¹H} NMR spectrum of the same copolymer (FIG. 12) allowed comparing the ratio between the PCHC and PLLA carbons. This ratio was dependent on the nature of the carbon atoms considered.

Table 7 shows the integral ratio between PCHC and PLLA carbons groups.

TABLE 7

| Carbon groups | | Integral ratio |
|---|---|---|
| PCHC (δ ppm) | PLLA (δ ppm) | (PCHC/PLLA)* |
| C═O (152.1-152.8) | C═O (168.6) | 0.67 |
| CH2 (21.1-28.7) | CH (68.0) | 0.58 |
| CH2 (21.1-28.7) | CH3 (15.6) | 0.51 |

*Calculated from the integrals of the ¹³C{¹H} NMR resonances in the PCHC-b-PLLA polymer and the relation: (integral of PCH carbons/number of carbons)/(integral of PLLA carbons/number of carbons).

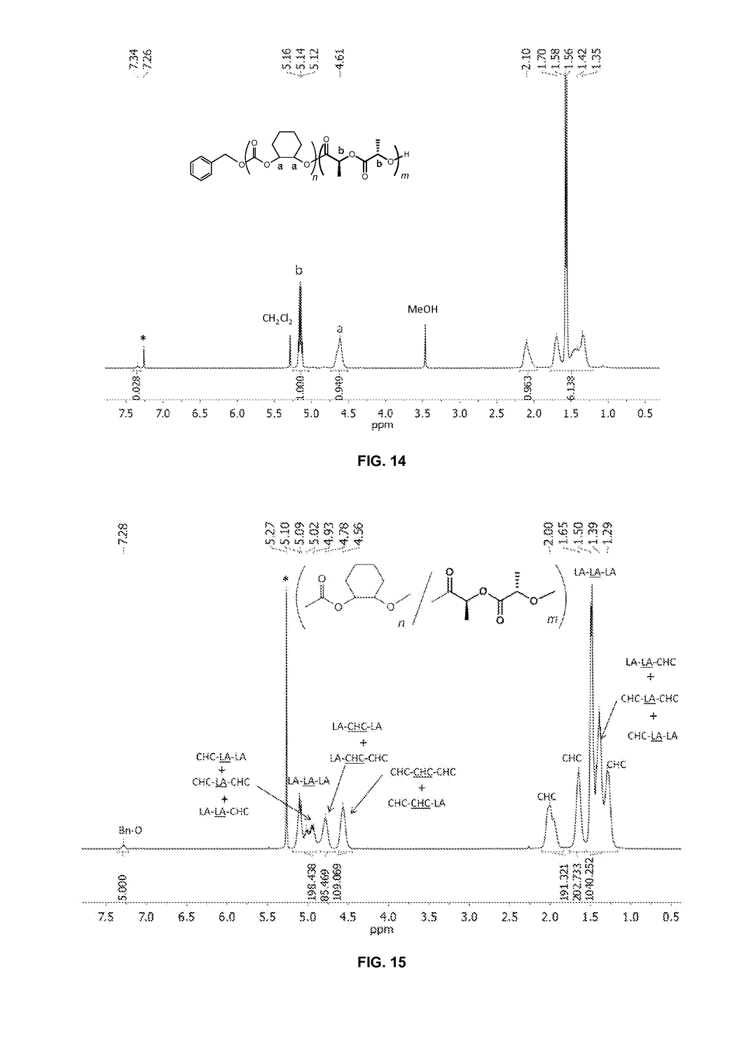

The same experiment (conditions of Table 6, entry 2) was repeated without any aliquot being taken out for analysis during the copolymerization (Table 6, Entry 2′). The ¹H NMR of the crude product (FIG. 13) exhibits a rac-CHC and LLA conversion of 86 and 95%, respectively. After precipitation of the copolymer, the ¹H NMR of the recovered sample showed (FIG. 14) that the integration of the signals corresponding to PCHC (CH at δ 4.61 ppm) and PLLA (CH at δ 5.06 ppm) are in a 0.95:1=0.95 ratio, which is in agreement with the ratio of the conversions (0.86:0.95=0.90).

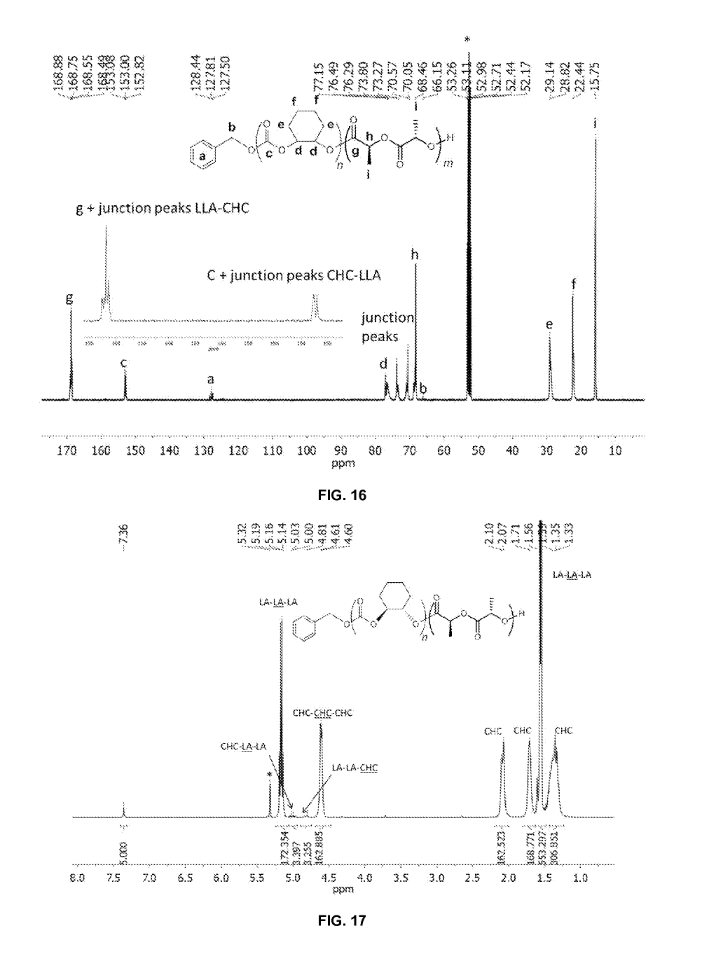

An experiment under the same operating conditions, yet with the reverse order of comonomers' addition (L-LA followed by rac-CHC) using [(NNO)ZnEt] catalyst was also performed. The ¹H and ¹³C{¹H} NMR (FIGS. 15 and 16) showed spectra similar to those of a random copolymer (as in FIGS. 9 and 10). This is most likely due to the trans-esterification of PLLA and/or PCHC.

### Example 9

### Preparation of PCHC-PLLA Diblock Copolymers from Sequential Copolymerization of (R,R)—CHC and L-Lactic Acid

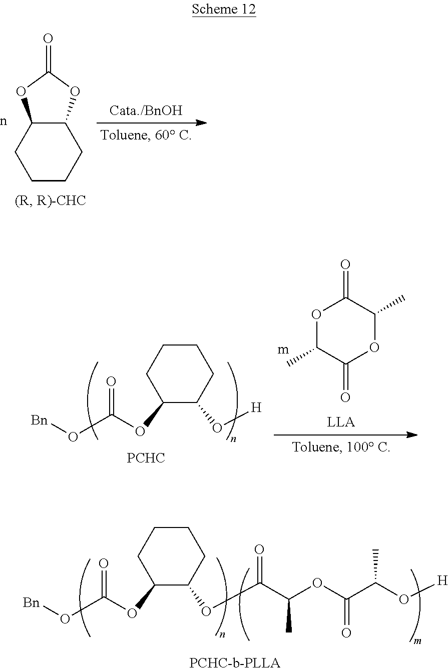

The process was performed by sequential copolymerization, wherein the (R,R)-cyclohexene carbonate ((R,R)—CHC) was added prior to addition of L-lactic acid (LLA). Different copolymerizations were performed using [(NNO)ZnEt] and benzyl alcohol (BnOH), as depicted in Scheme 12. The conditions and results are listed in Table 8. Typical procedure was as described in Example 8.

TABLE 8

| | | (R, R)- | | | | | | | |
|---|---|---|---|---|---|---|---|---|---|
| | [(R,R)-CHC]₀: | CHC^{a} | LLA^{a} | PCHC | PCHC | PCHC-b-PLLA | | | |
| | [LLA]₀: | Conv. | Conv. | M_{n,theo}^{b} | M_{n,NMR} | M_{n,theo}^{c} | M_{n,NMR}^{d} | M_{n,SEC}^{e} | |
| Entry | [Catal.]₀:[BnOH]₀ | (%) | (%) | (g/mol) | (g/mol) | (g/mol) | (g/mol) | (g/mol) | [symbol] M |
| 1 | 50:150:1:1 | 97 | 100 | 7000 | 8100 | 28 600 | 20 800 | | |
| 2 | 100:100:1:1 | 97 | 100 | 13 900 | 14 600 | 28 300 | 24 700 | | |
| 3 | 150:50:1:1 | 97 | 100 | 20 800 | 17 000 | 28 000 | 24 600 | | |

^{a}Calculated from the ¹H NMR of the crude product.
^{b}Theoretical molar mass value of PCHC calculated from the relation: M_{n,theo} = M_{(R,R)-CHC} × [(R,R)-CHC]₀/[ROH]₀ × conversion(_{R,R)-CHC} + M_{ROH} with M_{(R,R)-CHC} = 142 g · mol⁻¹, M_{BnOH} = 108 g · mol⁻¹.
^{c}Theoretical Mₙ value of PCHC-b-PLLA calculated from the relation: M_{n,theo} = M_{(R,R)-CHC} × [(R,R)-CHC]₀/[ROH]₀ × conversion _{(R,R)-CHC} + M_{LLA} × [LLA]₀/[ROH]₀ × conversion_{LLA} + M_{BnOH}.
^{d}NMR molar mass value of PCHC/copolymer calculated from the integral value ratio of the signals of alkoxide methylene or methine end-group hydrogens to internal methine hydrogens.
^{e}Experimental number average molar mass and dispersity values determined by SEC in THF at 30° C. using polystyrene standards.

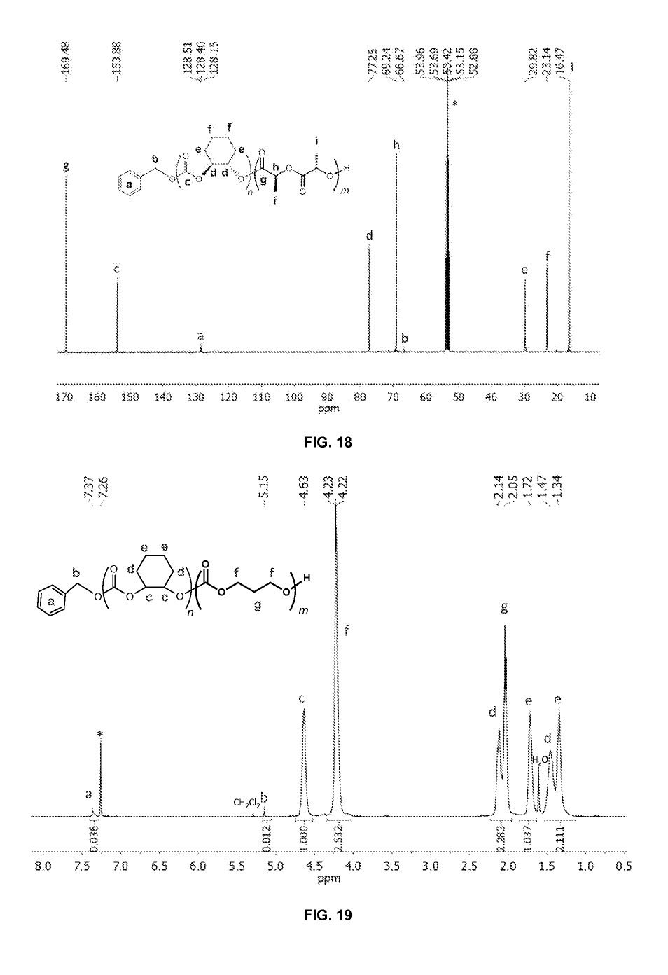

The PCHC-b-PLLA copolymer (Table 8, entry 8) was characterized by its ¹H NMR (FIG. 17) and ¹³C{¹H} NMR (FIG. 18) spectra.

### Example 10

### Preparation of PCHC-PTMC Copolymers

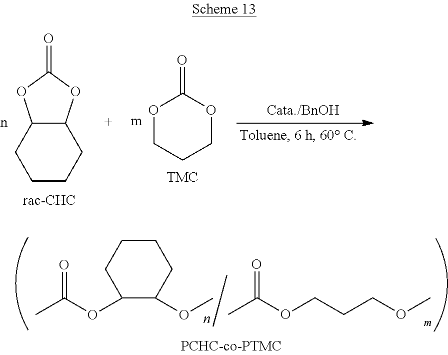

The copolymerization of racemic cyclohexene carbonate (rac-CHC) and trimethylene carbonate (TMC) was performed as depicted in Scheme 13. All polymerizations were performed similarly according to the following typical procedure (Table 9, entry 2). The only differences were in the nature of the catalyst and the initial concentrations of the monomers. The conditions and results are listed in Table 9. The copolymerizations were performed using the following catalytic systems: [(NNO)ZnEt] or TBD. The TMC (1,3-dioxane-2-one from Labso Chimie Fine, Blanquefort, France) was purified by first dissolving it in THF, stirring over CaH₂ for 2 days before being filtrated and dried in vacuo, and finally recrystallized from cold THF.

Typical procedure of CHC/TMC simultaneous copolymerization was as follows. In a dry-box, benzyl alcohol (0.97 μL, 9.34 μmol), [(NNO)ZnEt] (4.0 mg, 9.34 μmol) and dry toluene (0.10 mL) were placed in a Schlenk flask equipped with a magnetic stir-bar. The mixture was stirred 10 min at room temperature to form the corresponding alkoxide catalyst. rac-CHC (0.133 g, 0.934 mmol, prepared as in Example 3), TMC (0.095 g, 0.934 mmol, and dry toluene (0.37 mL) were then added. The flask and its contents were placed under argon and immersed in an oil-bath at 60 C. After stirring over 6 h, the reaction mixture was cooled down to room temperature and three drops of 1.6 M acetic acid solution in toluene were added to quench the reaction. The solvent was evaporated and the crude polymer was dissolved in CH₂Cl₂ (10 mL) and precipitated in cold methanol (50 mL). This process was repeated twice to ensure complete removal of the catalyst and the unreacted monomers. A white polymer was finally obtained following drying under vacuum.

TABLE 9

| | | | CHC | TMC | | | | |
|---|---|---|---|---|---|---|---|---|
| | | [rac-CHC]₀:[TMC]₀: | Conv.^{a} | Conv.^{a} | Mₙₜₕ^{b} | M_{nNMR}^{c} | M_{nSEC}^{d} | |
| Entry | Catalyst | [Cat.]₀:[BnOH]₀ | ( %) | ( %) | (g/mol) | (g/mol) | (g/mol) | [symbol] M |
| 1 | [(NNO)ZnEt] | 50:150:1:1 | 80 | 98 | 20 800 | 20 200 | 17 500 | 1.5 |
| 2 | [(NNO)ZnEt] | 100:100:1:1 | 80 | 98 | 20 800 | 18 900 | 14 100 | 1.7 |
| 3 | [(NNO)ZnEt] | 150:50:1:1 | 70 | 97 | 20 000 | 15 700 | 13 600 | 1.5 |
| 4 | TBD | 50:150:1:1 | 93 | 100 | 22 000 | 26 800 | 15 900 | 1.5 |
| 5 | TBD | 100:100:1:1 | 91 | 100 | 23 200 | 29 300 | 17 500 | 1.4 |
| 6 | TBD | 150:50:1:1 | 85 | 100 | 23 300 | 11 500 | 15 300 | 1.4 |

^{a}Calculated from the ¹H NMR of the crude product.
^{b}Theoretical Mₙ value of PCHC-co-PTMC calculated from the relation: M_{n,theo} = M_{rac-CHC} × [rac-CHC]₀/[ROH]₀ × conversion_{rac-CHC} + M_{TMC} × [TMC]₀/[BnOH]₀ × conversion_{TMC} + M_{BnOH}.
^{c}NMR molar mass value of PCHC calculated from the integral value ratio of the signals of alkoxide methylene end-group hydrogens to internal methine hydrogens.
^{d}Experimental number average molar mass and dispersity values determined by SEC in THF at 30° C. using polystyrene standards.

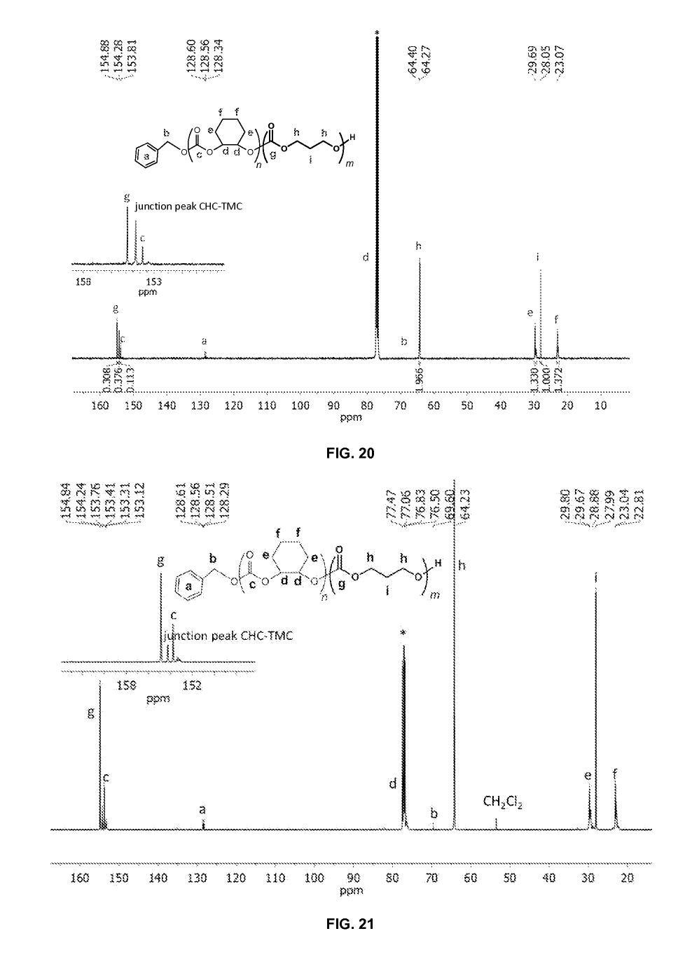

The ¹H NMR of the polymer produced in Entry 2 of Table 9 (FIG. 19), shows that the ratio of the integral values of signals of the PCHC (CH at δ 4.63 ppm) and the TMC (CH₂—O at δ 4.22 ppm) main chain methine and methylene, respectively, is equal to [(1/2)/(2.5/4)]=0.80, which is similar to the CHC and TMC conversions ratio (0.80:0.98=0.82; Table 5, entry 2). Analysis of the ¹³C{¹H} NMR of the same polymer (FIG. 20) (relaxation delay=10 s) exhibits the methylene groups of PCHC and PTMC in a 1.35:2=0.68 ratio, which is different from the ¹H NMR (0.80) as ¹³C{¹H} NMR is not a quantitative technique for these polymers.

### Example 11

### Preparation of PCHC-PTMC Copolymers by Sequential Copolymerization of Rac-CHC and TMC

A similar procedure as that described in Example 8 for the CHC/L-LA sequential copolymerization (PCHC-b-PLLA), was used for the preparation of PCHC-PTMC copolymers by sequential copolymerization of racemic cyclohexene carbonate (rac-CHC) and trimethylene carbonate (TMC). The process was performed by sequential copolymerization (addition of TMC prior to rac-CHC) at 100° C. in toluene. TMC was fully converted before addition of CHC. The copolymerizations were performed using the following catalytic system: [(NNO)ZnEt]. The conditions and results are listed in Table 10.

TABLE 10

| | | [TMC]₀: | TMC | CHC | PTMC | PTMC-b-PCHC | | | |
|---|---|---|---|---|---|---|---|---|---|
| | | [rac-CHC]₀: | Conv.^{a} | Conv.^{a} | Mnₜₕ^{b} | Mnₜₕ^{c} | Mn_{NMR}^{d} | Mn_{SEC}^{e} | |
| Entry | Catalyst | [Catal.]₀:[BnOH]₀ | (%) | (%) | (g/mol) | (g/mol) | (g/mol) | (g/mol) | [symbol] _{M} |
| 1 | [(NNO)ZnEt] | 50:150:1:1 | 100 | 62 | 5200 | 18 400 | 19 400 | 16 500 | 1.4 |
| 2 | [(NNO)ZnEt] | 100:100:1:1 | 100 | 83 | 10 300 | 21 800 | 26 800 | 20 300 | 1.6 |

^{a}Calculated from the ¹H NMR of the crude product.
^{b}Theoretical Mₙ value of PTMC calculated from the relation: M_{n,theo} = M_{TMC} × [TMC]₀/[BnOH]₀ × conversion_{TMC} + M_{BnOH} with M_{TMC} = 102 g · mol⁻¹, M_{BnOH} = 108 g · mol⁻¹.
^{c}Theoretical Mₙ value of PTMC-co-PCHC calculated from the relation: M_{n,theo} = M_{rac-CHC} × [rac- CHC]₀/[BnOH]₀ × conversion_{rac-CHC} + M_{TMC} × [TMC]₀/[BnOH]₀ × conversion_{TMC} + M_{BnOH}.
^{d}NMR molar mass value of PTMC-co-PCHC copolymer calculated from the integral value ratio of the signals of benzylalkoxide methylene end-group hydrogens to internal methine hydrogens.
^{e}Experimental number average molar mass and dispersity values determined by SEC in THF at 30° C. using polystyrene standards.

The ¹³C NMR spectrum of the copolymer entry 2 of Table 10, (FIG. 21), showed the same spectrum as the PCHC-co-PTMC random copolymer (FIG. 20). This most likely results from extensive trans-esterification of the PTMC and/or PCHC blocks under the reaction conditions.

### Example 12

### Preparation of PCHC-PTMC Diblock Copolymers by Sequential Copolymerization of Rac-CHC and TMC

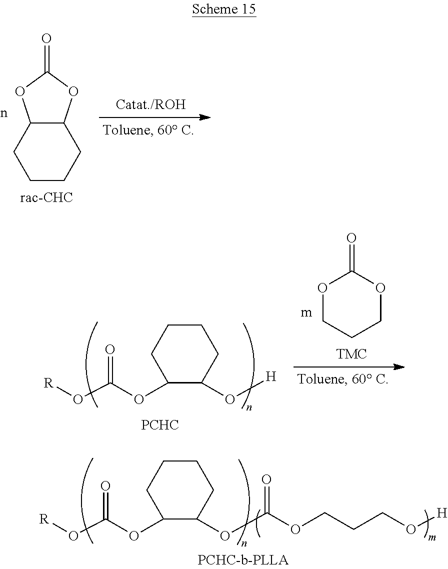

A similar procedure as that described in Example 8 for the CHC/L-LA sequential copolymerization (PCHC-b-PLLA), was used for the preparation of PCHC-PTMC diblock copolymers by sequential copolymerization of racemic cyclohexene carbonate (rac-CHC) and trimethylene carbonate (TMC). The process was performed by sequential copolymerization (addition of rac-CHC prior to TMC) at 60° C. in toluene as depicted in scheme 15, using various catalysts and different initial concentrations of TMC and rac-CHC. The conditions and results are listed in Table 11. The copolymerizations were performed using the following catalytic systems: [(NNO)ZnEt] and TBD.

TABLE 11

| | | [rac-CHC]₀: | CHC | TMC | | | | |
|---|---|---|---|---|---|---|---|---|
| | | [TMC]₀ | Conv.^{a} | Conv.^{a} | Mnₜₕ^{b} | Mn_{NMR}^{c} | Mn_{SEC}^{d} | |
| Entry | Catalyst | [Catal.]₀:[BnOH]₀ | (%) | (%) | (g/mol) | (g/mol) | (g/mol) | [symbol] _{M} |
| 1^{d} | [(NNO)ZnEt] | 50:150:1:1 | 96 | 100 | 22 200 | 16 800 | | |
| 2^{d} | [(NNO)ZnEt] | 100:100:1:1 | 96 | 100 | 24 000 | 18 600 | | |
| 3^{d} | [(NNO)ZnEt] | 150:50:1:1 | 96 | 100 | 25 700 | 22 600 | | |
| 4^{e} | TBD | 50:150:1:1 | 93 | 100 | 22 000 | 16 500 | | |
| 5^{e} | TBD | 100:100:1:1 | 92 | 100 | 23 400 | 21 500 | | |
| 6^{e} | TBD | 150:50:1:1 | 91 | 100 | 24 600 | 22 400 | | |

^{a}Calculated from the ¹H NMR of the crude product.
^{b}Theoretical Mₙ value of PCHC-b-PTMC calculated from the relation: M_{n,theo} = M_{rac-CHC} [rac-CHC]₀/[BnOH]₀ × conversion_{rac-CHC} + M_{TMC} [TMC]₀/[BnOH]₀ × conversion_{TMC} + M_{BnOH}.
^{c}NMR molar mass value of the copolymer calculated from the integral value ratio of the signals of benzylalkoxide methylene end-group hydrogens to internal methine hydrogens.
^{d}Experimental number average molar mass and dispersity values determined by SEC in THF at 30° C. using polystyrene standards.
^{d,e}The homopolymerization reaction time of rac-CHC is 6 h and 24 h, respectively.

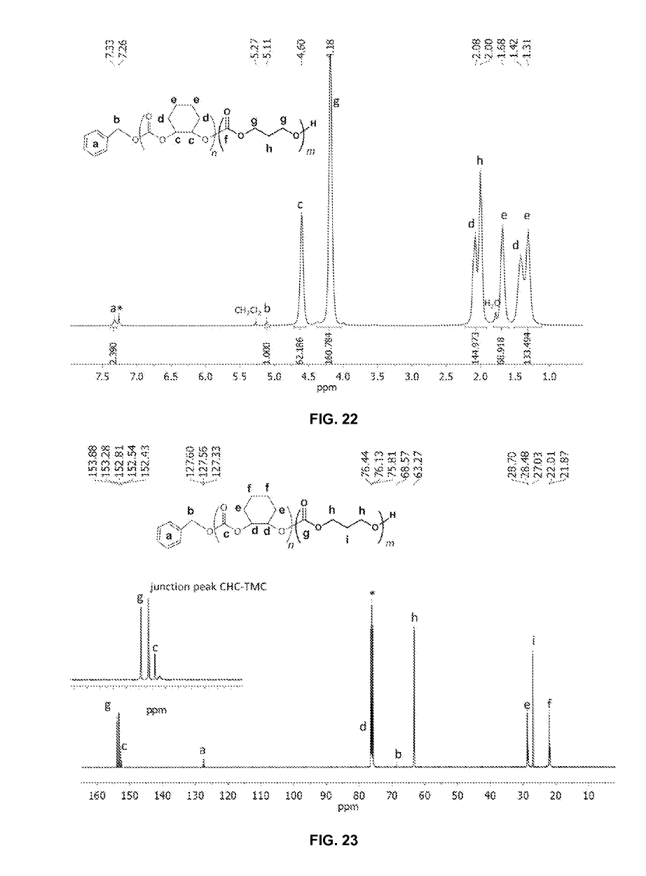

FIGS. 22 and 23 show respectively the ¹H NMR spectrum and 14C NMR spectrum of the copolymer entry 2 of Table 11.

1-15. (canceled)

16. A process for preparing a polycarbonate or copolymer thereof, said process comprising homo- or copolymerizing in the presence of at least one catalyst, one or more compounds of formula (I), stereoisomers, racemics, or mixtures thereof; optionally with one or more cyclic esters, stereoisomers, racemics, or mixtures thereof; 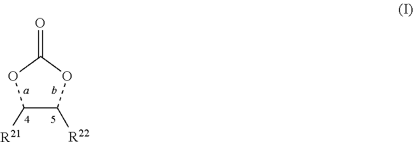
wherein each dotted bond “a” and “b” independently represents a solid bond, a wedged bond, or a hashed wedged bond; and
wherein R²¹ and R²² taken together with the carbon atoms 4 and 5 of the cyclic carbonate form a ring selected from C₃₋₉cycloalkylene, C₅₋₉cycloalkenylene, and C₆₋₁₂ arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

17. The process according to claim 16, wherein the catalyst is an organometallic catalyst of formula (II) or (III), 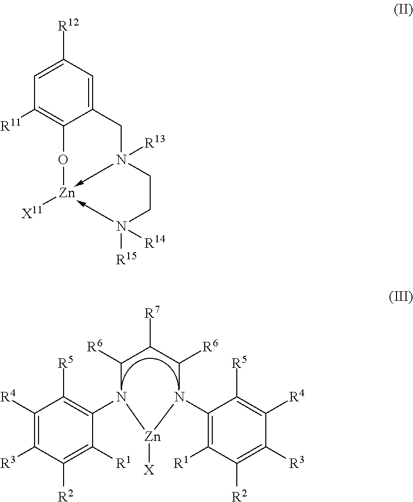
wherein R¹, R², R³, R⁴, R⁵, R⁶, and R⁷ are each independently selected from the group consisting of hydrogen, optionally substituted C₁₋₁₂alkyl, and an inert functional group;
wherein X is —N(SiR²⁷₃)₂, C₁₋₁₂alkyl, C₁₋₁₂alkoxy, —NR⁹R¹⁰ or —BH₄;
wherein each R²⁷ is independently selected from hydrogen and C₁₋₆alkyl;
wherein each R⁹ and R¹⁰ is independently selected from hydrogen and C₁₋₆alkyl;
wherein R¹ and R¹² are each independently C₁₋₁₀alkyl;
wherein R¹³, R¹⁴, and R¹⁵ are each independently C₁₋₁₀alkyl, or wherein R¹³ and R¹⁴ are covalently bound to each other and are each a methylene and R¹⁵ is C₁₋₁₀alkyl;
wherein X¹¹ is selected from C₁₋₁₀alkyl, —OR¹⁶, and —N(SiR¹⁷₃)₂;
wherein R¹⁶ is C₁₋₁₀alkyl; and
wherein each R¹⁷ is independently selected from hydrogen and C₁₋₆alkyl.

18. The according to claim 17, wherein the catalyst is selected from [(NNO)ZnEt], [BDI]Zn(N(SiMe₃)₂), [BDI]Zn(Et), and {[BDI]Zn(OR³⁰)}₂, wherein R³⁰ is C₁₋₆alkyl, and wherein [BDI]Zn(N(SiMe₃)₂) and [(NNO)ZnEt] have the following structures:

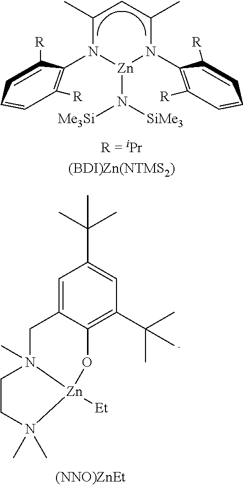

19. The process according to claim 16, wherein the catalyst is selected from complexes of formulae M(OSO₂CF₃)ₘ, M(N(OSO₂CF₃)₂)ₘ, M(R²³C(O)CR²³₂C(O)R²³)ₘ, and (R²⁴CO₂)ₘM, wherein M is a metal of Group 2, 3, including the lanthanide series, or Group 12, 13, 15, wherein each R²³ is independently an optionally substituted C₁₋₁₂ alkyl, wherein each R²⁴ is independently a perfluorinated C₁₋₁₂alkyl or an aryl, and wherein m is the valence of M.

20. The process according to claim 16, wherein the catalyst is selected from the group consisting of dimeric phosphazene bases, or amines or guanidines, organic acids, sparteine, thiourea-amino derivatives, N-heterocyclic carbenes, 4-(dialkylamino)pyridines, amidines, phosphorus based phosphines, phosphazenium derivatives and phosphazenes, metal amides; and compounds of general formula M¹(Y¹, Y², . . . Y^{p})^{q}, in which M¹ is a metal selected from the group consisting of the elements of columns 3 to 12 of the periodic table of the elements, and the elements Al, Ga, In, Tl, Ge, Sn, Pb, Sb, Ca, Mg and Bi; wherein Y¹, Y2, . . . Y^{p} are each substituents selected from the group consisting of alkyls with 1 to 20 carbon atoms, aryls having from 6 to 30 carbon atoms, alkoxys having from 1 to 20 carbon atoms, aryloxys having from 6 to 30 carbon atoms, oxides, carboxylates, and halide groups, and wherein p and q are integers of from 1 to 6.

21. The process according to claim 20, wherein the catalyst is selected from the group consisting of 1,5,7-triazobicyclo-[4,4,0]dec-5-ene (TBD); 1,8-diazabicyclo[5.4.0]undec-7-ene (DBU); 4-dimethylaminopyridine (DMAP); tert-butylimino-1,3-dimethylperhydro-1,3,2-diazaphosphine (BEMP); Sn(Oct)₂; Al(OiPr)₃; Ti(OiPr)₄; Ti(2-ethylhexanoate)₄; Ti(2-ethylhexyloxide)₄; Zr(OiPr)₄; Bi(neodecanoate)₃; (2,4-di-tert-butyl-6-(((2-(dimethylamino)ethyl)(methyl)amino)methyl)phenoxy)(ethoxy)zinc; Zn(lactate)₂; Y[N(SiMe₃)₂]₃; [Y]+L1; and [Y]+L2, wherein L1 is a tetradentate ligand of formula L1 and L2 is a tetradentate ligand of formula L2:

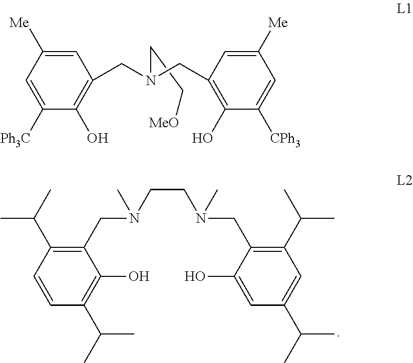

22. The process according to claim 16, wherein the process is performed in the presence of a compound of formula (IV),
R⁸—OH (IV)
wherein R⁸ is selected from the group consisting of C₁₋₂₀alkyl, C₆₋₃₀aryl, and C₆₋₃₀arylC₁₋₂₀alkyl optionally substituted by one or more substituents selected from the group consisting of halogen, hydroxyl, and C₁₋₆alkyl.

23. The process according to claim 16, wherein the homo- or copolymerizing is in the presence of cyclic esters, wherein the cyclic esters are selected from the group consisting of lactones, cyclic carbonates, glycolides and lactides, wherein each group is unsubstituted or substituted with one or more substituents each independently selected from C₁₋₆alkyl and C₁₋₆alkoxy.

24. The process according to claim 16, wherein the homo- or copolymerizing is in the presence of cyclic esters, wherein the cyclic esters are selected from the group consisting of lactide; trimethylene carbonate; glycolide; β-butyrolactone; δ-valerolactone; γ-butyrolactone; γ-valerolactone; 4-methyldihydro-2(3H)-furanone; alpha-methyl-gamma-butyrolactone; ε-caprolactone; 1,3-dioxolan-2-one; propylene carbonate; 4-methyl-1,3-dioxan-2-one; 1,3-doxepan-2-one; 5-C₁₋₄alkoxy-1,3-dioxan-2-one; and mixture thereof, wherein each group is unsubstituted or substituted with one or more substituents each independently selected from C₁₋₆alkyl and C₁₋₆alkoxy.

25. The process according to claim 16, wherein the process is performed in the presence or absence of a solvent.

26. The process according to claim 16, wherein the polycarbonate or copolymer thereof comprises recurring units of formula (VII3) or of formula (VII4),

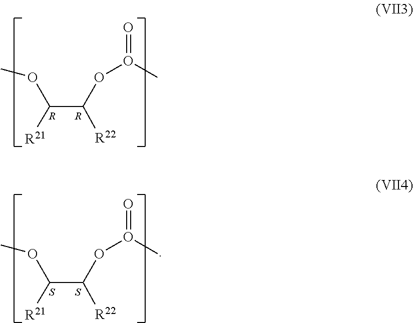

27. An article comprising a polycarbonate or copolymer thereof prepared according to claim 16.

28. A process for preparing a polycarbonate or copolymer thereof, said process comprising copolymerizing in the presence of at least one catalyst, one or more compounds of formula (I), stereoisomers, racemics, or mixtures thereof; with one or more cyclic esters, stereoisomers, racemics, or mixtures thereof; 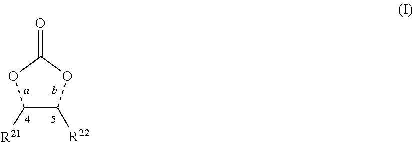
wherein each dotted bond “a” and “b” independently represents a solid bond, a wedged bond, or a hashed wedged bond; and
wherein R²¹ and R²² taken together with the carbon atoms 4 and 5 of the cyclic carbonate form a ring selected from C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

29. The process according to claim 28, wherein two of said one or more substituents of the ring formed by R²¹ and R²² with the carbon atoms 4 and 5 are joined to form a ring fused to said C₃₋₉cycloalkylene, C₅₋₉cycloalkenylene, or C₆₋₁₂arylene;
wherein said ring formed by said two substituents is selected from the group consisting of C₃₋₉cycloalkyl, C₅₋₉cycloalkenyl, C₆₋₁₂aryl, and heterocyclyl, wherein each group is unsubstituted or substituted with one or more R⁴⁰, wherein each R⁴⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

30. An article comprising a polycarbonate or copolymer thereof prepared according to claim 28.

31. A process for the synthesis of a compound of formula (I) a stereoisomer or racemate thereof, 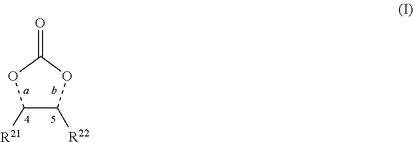
the process comprising:
contacting a compound of formula (V), a stereoisomer, or racemate thereof; with at least one reagent selected from the group consisting of an alkyl- or cycloalkyl-chloroformate of formula (VI), wherein formulas (V) and (VI) are: 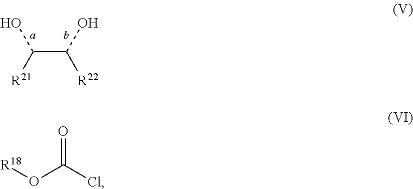
wherein each dotted bond “a” and “b” independently represents a solid bond, a wedged bond, or a hashed wedged bond;
wherein R²¹ and R²² taken together with the carbon atoms to which they are attached form a ring selected from the group consisting of C₃₋₉cycloalkylene, C₅₋₉cycloalkenylene, and C₆₋₁₂arylene, wherein the ring is optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; and
wherein R¹⁸ is C₁₋₆alkyl, or C₃₋₆cycloalkyl.

32. A polycarbonate or copolymer thereof comprising a recurring unit of formula (VII1) and/or (VII2), 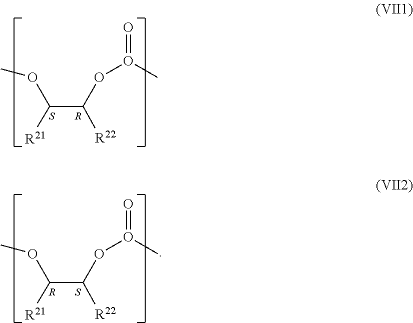
wherein R²¹ and R²² taken together with the carbon atoms to which they are attached form a ring selected from C₃₋₉cycloalkylene, C₅₋₉cycloalkenylene, and C₆₋₁₂arylene, said ring being optionally substituted with one or more substituents each independently selected from the group consisting of halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino.

33. The polycarbonate or copolymer thereof according to claim 32, comprising recurring units each independently represented by the following formula (VIIA) or (VIIB) or (VIIC); 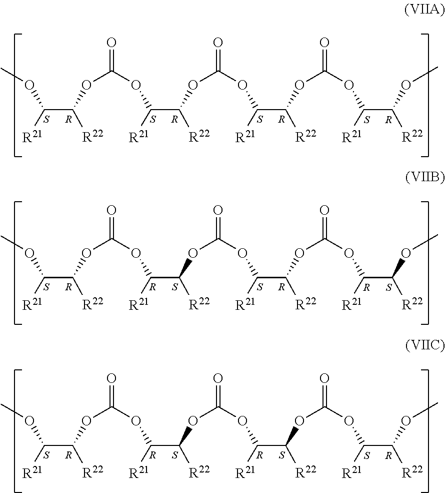
wherein R²¹ and R²² have the same meaning as defined in claim 32.

34. The polycarbonate or copolymer thereof according to claim 32, comprising recurring units each independently represented by the following formula (VIIA1) or (VIIB1) or (VIIC1); 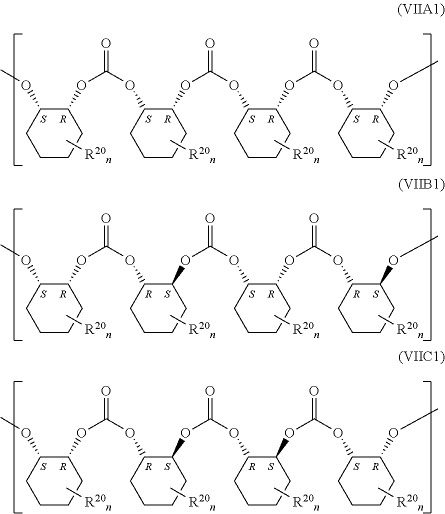
wherein each R²⁰ is independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino;
wherein n is an integer selected from 0, 1, 2, 3, or 4.

35. An article comprising a polycarbonate or copolymer thereof according to claim 32.

36. A polycarbonate or copolymer thereof comprising recurring units of formula (VII3) or of formula (VII4), 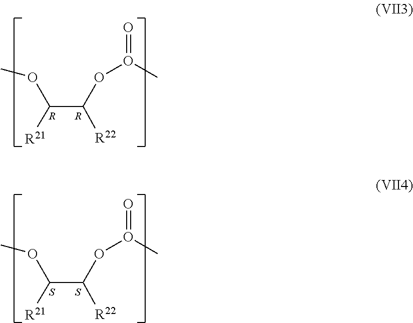
wherein R²¹ and R²² taken together with the carbon atoms 4 and 5 to which they are attached form a ring selected from the group consisting of C₃₋₉cycloalkylene, C₅₋₉cycloalkenylene, and C₆₋₁₂arylene, said ring being optionally substituted with one or more substituents each independently selected from halo, hydroxyl, C₁₋₆alkyl, haloC₁₋₆alkyl, C₁₋₆alkoxy, haloC₁₋₆alkoxy, C₆₋₁₂aryl, C₁₋₆alkylthio, carboxyl, C₁₋₆alkoxycarbonyl, C₁₋₆alkylcarbonyloxy, amino; or mono- or di-C₁₋₆alkylamino; and
wherein said polycarbonate or copolymer thereof is characterized by a ¹³C{¹H} NMR spectrum comprising only one signal present in the areas ranging from δ 150 to 155 ppm.

37. An article comprising a polycarbonate or copolymer thereof according to claim 36.
